SEQUENCE LISTING (8 sequences)

SEQ ID NO: 1 - DNA, 16 bp, Artificial Sequence
  Oligonucleotide
tagtagccatgaattc

SEQ ID NO: 2 - DNA, 88 bp, Artificial Sequence
  Detection molecule
  misc_feature 40..48: /note=“n” means any nucleotide“
  misc_feature 88: /note=”blocked 3′ OH“
gtacagtatagccagcactacattcgacgtacagtacgannnnnnnnngttagttacagtttaccgatactagaattcatggctacta

SEQ ID NO: 3 - DNA, 44 bp, Artificial Sequence
  Extension oligonucleotide
  misc_feature 14..23: /note=”n“ means any nucleotide; indicated as ”x“ in Fig. 5”
  misc_feature 44: /note=“blocked 3′ OH”
gtacatgcatgacnnnnnnnnnnctagtatcggtaaactgtaac

SEQ ID NO: 4 - DNA, 76 bp, Artificial Sequence
  Cleaved barcode label
  misc_feature 40..51: /note=“n” means any nucleotide“
gtacagtatagccagcactacattcgacgtacagtacgannnnnnnnnnnngttagttacagtttaccgatactag

SEQ ID NO: 5 - DNA, 12 bp, Artificial Sequence
  Universal primer 1
gtacagtatagc

SEQ ID NO: 6 - DNA, 13 bp, Artificial Sequence
  Universal primer 2
gtacatgcatgac

SEQ ID NO: 7 - DNA, 100 bp, Artificial Sequence
  Barcode label 3′-5′
  misc_feature 40..51: /note=”n“ means any nucleotide”
  misc_feature 77..87: /note=“n” means any nucleotide; indicated as “y” in Fig. 5“
gtacagtatagccagcactacattcgacgtacagtacgannnnnnnnnnnngttagttacagtttaccgatactagnnnnnnnnnnngtcatgcatgtac

SEQ ID NO: 8 - DNA, 99 bp, Artificial Sequence
  Barcode label 5′-3′
  misc_feature 14..23: /note=”n“ means any nucleotide; indicated as ”x“ in Fig. 5”
  misc_feature 49..60: /note=“n” means any nucleotide“
gtacatgcatgacnnnnnnnnnnctagtatcggtaaactgtaactaacnnnnnnnnnnnntcgtactgtacgtcgaatgtagtgctggctatactgtac

(19) United States
(12) Patent Application Publication
(10) Pub. No.: US 2023/0357822 A1
(43) Pub. Date: Nov. 9, 2023

(54) METHOD FOR ANALYZING CELL RELEASED BIOMOLECULES

(71) Applicant: EVORION Biotechnologies GmbH, Münster (DE)

(72) Inventors: Robert WEINGARTEN, Münster (DE); Sebastian BÜHREN, Nottuln (DE); Hans KLEINE-BRÜGGENEY, Münster (DE)

(21) Appl. No.: 18/245,610

(22) PCT Filed: Sep. 17, 2021
(86) PCT No.: PCT/EP2021/075711

§ 371 Date: Mar. 16, 2023

Related U.S. Application Data
(60) Provisional application No. 63/079,990, filed on Sep. 17, 2020.

(51) Int. Cl. C12Q 1/6804

(52) U.S. Cl. CPC C12Q 1/6804

(57) ABSTRACT

The invention inter alia pertains to methods for the analysis of one or more cell released biomolecules.

### BACKGROUND OF THE INVENTION

Cells have been found to be phenotypically different, despite being genetically identical. Indeed, cell populations of seemingly same cells appear to always contain cells which are different from each other at a particular resolution of inspection (Alterschule and Wu, 2010, Cell, 141(4), pages 559-563). Standard assays are only capable of determining average responses of the cells, which is interpreted as the response of all cells in that sample. Specialized cells which exist in nearly all cell populations (e.g. cancer stem cells) are ignored in such bulk assays and valuable information about these cells is lost. Yet, the knowledge about individual cells is important to elucidate molecular mechanisms, which are for instance involved in cancer formation, ageing and immune responses. In order to understand and utilize the intercellular differences, for instance, for therapeutic purposes, there is a need for analysing cells on a single-cell level. Importantly, at a single-cell level, methods and techniques are required, which allow analysis of large numbers of single-cells in order to screen sufficiently large cell populations. Furthermore, these methods and techniques need to allow analysis of multiple phenotypic traits throughout the cellular cultivation. In addition, a sequential coupling of the determined phenotypic traits with genomic data is desirable.

With the aim to analyse molecules of single cells or small cell populations, an immunoassay, called FluoroSpot assay, has been developed (Janetzki et al., 2014, Cells, 2, pages 1102-1115). Therefore, molecule-specific capture antibodies (e.g. against cytokines) are added to an assay plate having wells. Afterwards, cells are added to the wells in suspension at a low dilution and the assay plate is incubated to allow for cytokine secretion by the cells. The secreted cytokines may be captured by the immobilized cytokine-specific capture antibodies at the bottom of wells. Thereafter, the cells may be removed and the wells may be washed and added with molecule-specific detection antibodies and fluorophore conjugates. Finally, secretory footprints (or spots) of the secreted molecules (e.g. cytokines) are captured by way of imaging, and such images are analysed for identifying multiple analytes, counting number of cells secreting the analytes, and the like. Another end point analysis of single cells has been developed to predict the response of cells to anti-PD-1 immunotherapy by single-cell mass cytometry. Therefore, metal labelled antibodies are used, which bind to cellular proteins, and analysed via time of flight mass spectrometry. A sequential data analysis sorts the detected signals for each cell, while the number of bound and labelled antibodies enables a quantitative analysis (Krieg et al., 2018, Nature Medicine, 24, pages 144-153).

In order to analyse multiple molecules secreted by single cells throughout the cultivation and acquire time-resolved information about the secreted molecules, Lu et al. developed a microfabricated device based fluorescence-barcoding technique (Lu et al., 2015, Proc. Natl. Acad. Sci. USA, 112(7), pages E607-E615): Statistically distributed, mostly single cells are captured in small PDMS compartments, which are then covered by an antibody-coated array. Similar to a barcode, antibodies are placed as fine lines onto the array, wherein one line corresponds to one type of antibody specific against a particular molecule (here cytokine). Throughout the cultivation, the single cells secrete cytokines, which are then captured by the specific antibody. After the cultivation time (e.g. 24 h), the array is removed and a sandwich assay is performed. This assay applies fluorescently labelled detection antibodies to the array, which bind to a different epitope of the bound target cytokines for staining (up to 3 colours). Afterwards, imaging can be conducted, which reveals in case of a secreted cytokine a coloured spot, whereas no colour can be detected when the cytokine is not detected. The advantage of this technology is a multiplexed analysis of statistically-distributed single cells.

Moreover, up to 12,000 PDMS compartments can be present in one microfabricated device, allowing a highly multiplexed analysis (Xue et al., 2017, Journal for ImmunoTherapy of Cancer, 5(1):85). Yet, there is a need for methods that improve analysis of multiple molecules secreted by single cells.

Moreover, it is desirable to study the interaction of two or more cells or cell types in a defined manner. For instance, priming immune cells against certain cancer cells has proven to be an effective strategy in inhibiting cancer propagation and is envisioned in form of cancer immunotherapy as the next step in cancer treatment (Steer et al., 2010, Oncogene, 29, pages 6301-6313). Therefore, methods and techniques are required, which enable to place a defined number of (different) cells next to each other in a closed compartment. Furthermore, interfaces should be present that allow integration of methods for multiplexed analysis of secreted molecules to study the cellular interaction. Noteworthy, analysis of single cells or cellular interactions should allow transferring the gained insights to in vivo conditions. This requires establishing an environment for the cells which mimics the conditions the cells encounter in the body.

The invention aims at avoiding drawbacks of the prior art methods. In particular, it is an object to provide further methods for analysing one or more biomolecules released by at least one cell, in some cases exactly one cell, wherein said methods shall also enable the analysis in a multiplex fashion.

### SUMMARY OF THE INVENTION

The present invention relates to the analysis of cell-released biomolecules. The present invention provides different methods that also allow the analysis and detection of multiple biomolecules of interest, wherein the cells that release biomolecules are provided in an environment that is capable of mimicking the native cell environment.

As described herein, the present invention provides a method for analyzing one or more cell released biomolecules, comprising providing a cell-laden matrix, wherein the cell-laden matrix comprises at least one cell that releases one or more biomolecules of interest or wherein the cell-laden matrix comprised at least one cell that has released one or more biomolecules of interest and wherein the method comprises the following steps:
a) providing a capture matrix, wherein the capture matrix comprises one or more types of capture molecules, wherein each type of capture molecule binds a biomolecule of interest,
b) incubating the cell-laden matrix to allow release of the one or more biomolecules of interest and binding the one or more biomolecules of interest to the one or more types of capture molecules of the capture matrix thereby providing a loaded capture matrix;
c) optionally further processing the loaded capture matrix;
d) using one or more types of detection molecules comprising a barcode label which comprises a barcode sequence (B_{S}) indicative for a target biomolecule of interest for generating an amplifiable molecule that comprises the barcode sequence (B_{S}) and/or reverse complement thereof.

The following figures illustrate non-limiting examples of the method of the present invention. In particular, different advantageous strategies for assigning the barcode label of a detection molecule to a target biomolecule of interest are described that allow the analysis of cell released biomolecules. These different strategies inter alia involve the use of an endonuclease, a proximity extension assay, the use of complex barcode labels and/or sequential labelling strategies. Other objects, features, advantages and aspects of the present application will become apparent to those skilled in the art from the following description and appended claims. It should be understood, however, that the following description, appended claims, and specific examples, while indicating preferred embodiments of the application, are given by way of illustration only.

In the subsequent figure description, inter alia the following terms are used which in preferred embodiments refer to the following elements:
Partition 1=In particular refers to a microfluidic cell culture chip located within an incubation chamber.
Partition 2=In particular refers to a microfluidic processing chip, preferably located within a XYZ-Pipetting Robot. Partition 2 is connected with partition 1 using tubing. Capture beads can be positioned in partition 2 and individually perfused with different solutions located in a separate partition (e.g. a 1536 well plate that contains reagents such as in embodiments, a conjugated-Ab library). Capture beads can be removed again and transferred into partition 3. As described herein, partition 2 is in certain embodiments not required.
Partition 3=In particular refers to a well of a microtiter plate or a reaction tube.

Linker=A linker in particular refers to molecule that is degradable on demand e.g. by enzymes, chemicals, heat, pressure, mechanical forces, light irradiation.

According to one embodiment of the present method, the barcode label of a detection molecule used in step d) comprises at least one nuclease target site (NTS) and an analyte specific sequence (ASS) that is specific for a biomolecule of interest. In this embodiment, step d) may comprise hybridizing the barcode label of the detection molecule to an oligonucleotide that is associated with the loaded capture matrix, wherein said associated oligonucleotide comprises
at least one nuclease target site (NTS′) that is complementary to the at least one nuclease target site (NTS) of the barcode label of the detection molecule, and
at least one analyte specific sequence (ASS′) that is complementary to the analyte specific sequence (ASS) of the barcode label of the detection molecule, whereby an at least partially double-stranded hybrid molecule is formed that comprises (i) at least one cleavable recognition site for an endonuclease that is provided by the hybridized nuclease target sites and (ii) the hybridized analyte specific sequences.

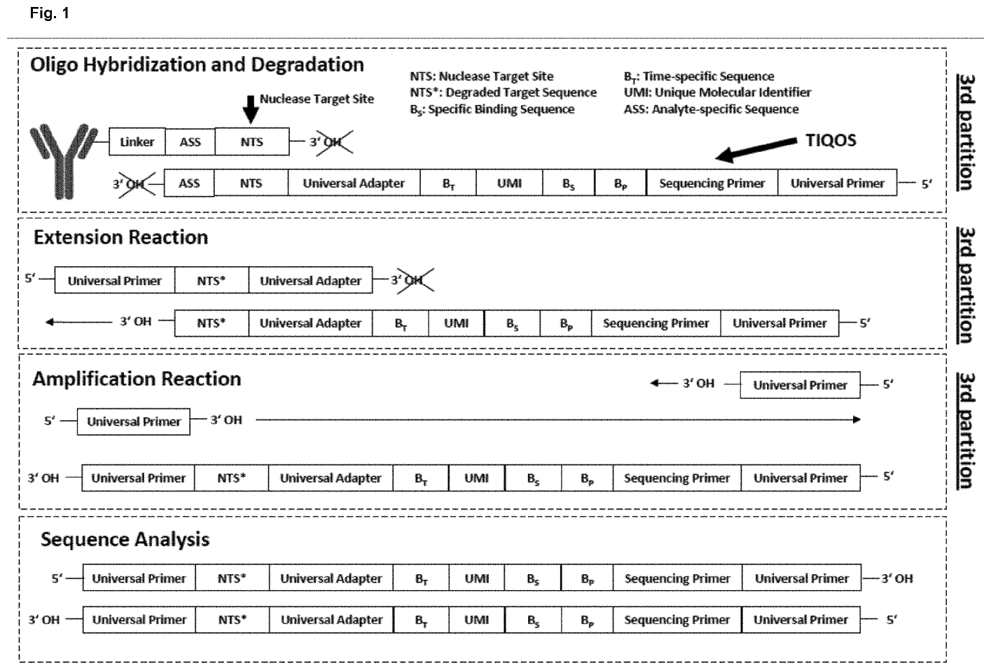

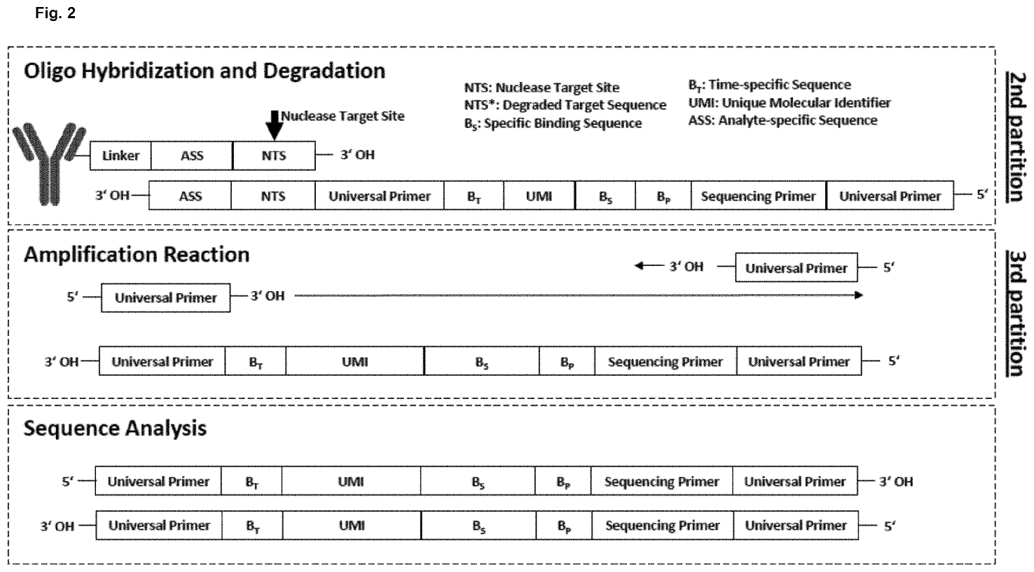

Advantageous embodiments of such method that involves the use of an endonuclease are disclosed in FIG. 1 to FIG. 6.

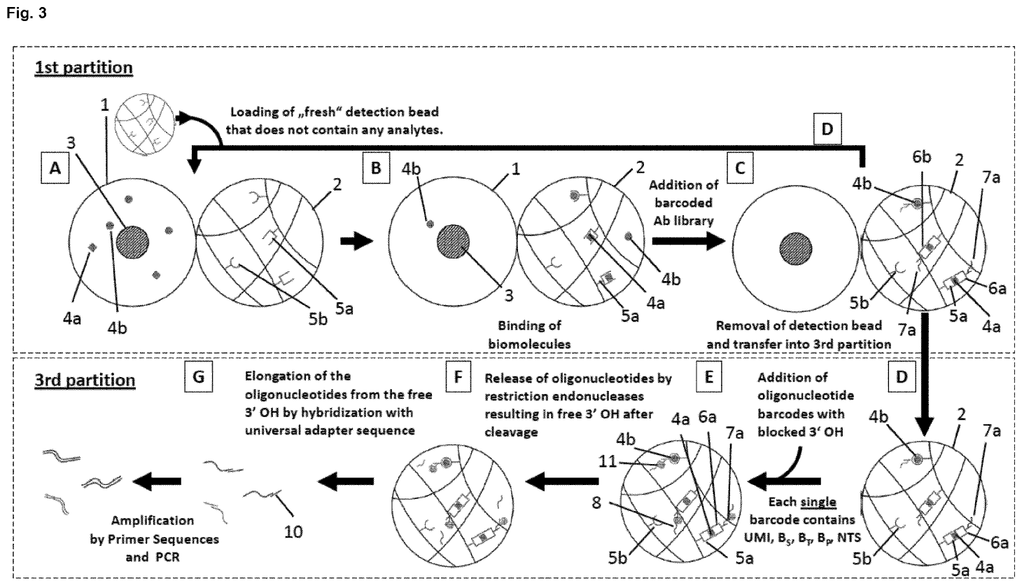

FIG. 1 discloses an embodiment of said method involving the use of an endonuclease wherein the oligonucleotide hybridization, the nuclease cleavage, the barcode label extension reaction and the amplification reaction may be performed in the same partition, such as a well of a microtiter plate or a reaction tube (also referred to as 3^{rd} partition herein). FIG. 3 provides a general overview over the procedure that may be used in this embodiment. In an advantageous embodiment, the method is performed as follows (as illustrated in FIG. 1 and FIG. 3).

A 1^{st} partition (e.g. provided in a microfluidic cell culture chip located within an incubation chamber), contains a cell laden matrix, a capture matrix (e.g. a capture bead) comprising one or more types of capture molecules and the released one or more target analyte(s) (biomolecule of interest). The capture molecule of the capture matrix may be provided by an antibody (as is illustrated in FIG. 3). The cell-laden matrix may comprise one or more cells or the cell-laden matrix comprised at least one cell that has released one or more analytes of interest (an empty matrix where one or more cells have been present—e.g. cell(s) may have undergone apoptosis or may escape from the matrix by cell migration). The capture molecules bind their analyte, thereby providing a loaded capture matrix. This loaded capture matrix is contacted with a secondary capture molecule (in FIG. 1 an antibody) that binds the target analyte that is bound to the loaded capture matrix and to which an oligonucleotide is attached (partition 1 preferred, partition 2 optionally). The oligonucleotide comprising (i) the at least one nuclease target site that is complementary to the at least one nuclease target site of the barcode label of the detection molecule and (ii) the at least one analyte specific sequence that is complementary to the analyte specific sequence of the barcode label of the detection molecule is attached to the secondary capture molecule. For the ease of simplicity, the nuclease target site (NTS) and the analyte specific sequence (ASS) comprised in the barcode label as well as the reverse complements thereof that are comprised in the oligonucleotide are shown as (NTS) and (ASS) in the Figures referring to these elements. In the embodiment illustrated in FIG. 1, the oligonucleotide has a blocked 3′OH end and is attached via a linker to the secondary capture molecule. Afterwards, the loaded capture matrix is preferably washed to remove any unbound secondary capture molecules (partition 1 preferred, partition 2 optionally).

Optionally, the capture matrix is then contacted e.g. by perfusion with one or more types of detection molecules, preferably a mixture of different types of detection molecules to be able to analyse, e.g. detect, two or more released target analytes. This step can be performed in a 2^{nd} Partition (e.g. in a microfluidic processing chip).

However, preferably, the loaded capture matrix comprising the bound secondary capture matrix with the attached oligonucleotide attached is transferred to a 3^{rd} partition (e.g. well of a microtiter plate or a reaction tube). In the 3^{rd} partition, the loaded capture matrix may be contacted e.g. by diffusion with one or more types of detection molecules, preferably a mixture of different types of detection molecules (partition 3, transfer of the loaded capture matrix comprising the bound secondary capture molecule from partition 1/2 can occur using RFCP geometry, said geometry being described elsewhere herein). As described herein in illustrated in FIG. 1, the detection molecule comprises or consists of a barcode label that has a region at its 3′ end that specifically hybridizes with the oligonucleotide that is attached to the secondary antibody. As shown in FIG. 1, the barcode label comprises a blocked 3′ OH end and therefore, cannot be extended by a polymerase. As is illustrated in FIG. 1, the detection molecule may also consist of the barcode label which is here provided by a single-stranded oligonucleotide.

The mixture comprising the loaded capture matrix with the bound secondary capture molecules (whereby the oligonucleotide attached to the secondary capture molecule becomes associated with the loaded capture matrix) may comprise one or more different types of detection molecules, preferably two or more different types of detection molecules.

According to a preferred embodiment, the detection molecules comprise barcode labels have the following characteristics:
All barcode labels share a common barcode sequence B_{P} that encodes a position information.
All barcode labels share a common barcode sequence B_{T} that encodes a time information.
The barcode labels of the same type of detection molecule have a common barcode sequence B_{S} and have a common analyte specific sequence (ASS), wherein said common analyte specific sequence (ASS) is capable of hybridizing with the oligonucleotides of secondary antibodies and a common analyte specific sequence (ASS).
All barcode labels have UMI sequences that are different for every individual barcode label or, in a preferred embodiment, all barcode labels have UMI sequences that are different for every individual barcode molecule of the same type of detection molecule that therefore, have a common barcodes sequence B_{S}.
All barcode labels have a common universal adapter sequence that provides a universal primer target sequence for the extension reaction.
All barcode labels comprise a common nuclease target site (NTS) that enables the specific degradation of barcode labels hybridized to the oligonucleotides attached to the secondary capture molecules using an endonuclease.
All barcode labels comprise at least one common universal primer target sequence (in short also referred to as primer sequence) that enables amplification of the barcode label.
Optionally, but preferably, all barcode labels comprise at least one common sequencing primer target sequence (in short also sequencing primer), that e.g. enables sequencing reactions with common platforms (e.g. Illumina, Pacific Bioscience, Nanopore).

As is shown in FIG. 1, the oligonucleotide attached to the secondary capture molecule and the barcode label hybridize to each other at the complementary analyte specific sequences and the nuclease target sites, whereby a double-stranded portion comprising at least one recognition site for an endonuclease is formed. The hybrid is then cleaved (“degraded”) using at least one endonuclease which specifically recognizes the cleavable recognition site that is formed in the double-stranded hybrid portion. The endonuclease does not cleave the single-stranded oligonucleotide or the single-stranded barcode label. Instead, it only cleaves if the double-stranded hybrid is formed to ensure that the single-stranded barcode label is not cleaved at the nuclease target site (NTS). The endonuclease is preferably a restriction endonuclease. Furthermore, as described herein, preferably multiple endonucleases are used to release the barcode label from the formed hybrid. Cleavage by the one or more endonucleases results provides a free 3′ OH after endonuclease cleavage. Optionally, the cleaved barcode label may be further processed, e.g. single nucleotides could be removed at the 3′ end, however, maintaining a free 3′ OH end. The cleaved and optionally further processed barcode label is extended in an extension reaction at the free 3′ OH end to provide an amplifiable molecule that comprises at least one sequence from the extension reaction. As is illustrated in FIG. 1, the amplifiable molecule is provided in the extension reaction by hybridizing an extension oligonucleotide to the cleaved and optionally further processed barcode label, wherein the extension oligonucleotide provides a template for the extension reaction. The generated amplifiable molecule preferably includes a universal primer target sequence. As is illustrated in FIG. 1, such universal primer target sequence may be added at the 3′ end of the barcode label, by hybridizing an extension oligonucleotide to the cleaved and optionally further processed barcode label, wherein the extension oligonucleotide provides the template for adding in the extension reaction the universal target primer sequence at the 3′ end of the barcode label (referred to as universal primer in FIG. 1). As shown in FIG. 1, the extension oligonucleotide preferably hybridizes to the universal adapter sequence and cleaved (degraded) nuclease target site of the cleaved barcode label and comprises a blocked 3′OH end. The extension oligonucleotide is used as template for the extension reaction, whereby a common primer sequence, preferably a universal primer target sequence, is added to all barcode labels, which is identical for all different barcode labels. The so extended barcode labels comprising the added primer target sequence can subsequently be amplified with a primer or primer pair.

In a further embodiment, the barcode sequences Bp and/or B_{T} are not directly present in the barcode label but are added during the amplification reaction, so that they are present in the sequenceable molecule.

The embodiment illustrated in FIG. 1 has several advantages. The barcode labels can only be amplified after being cleaved and thus degraded by an endonuclease and thus after having hybridized to an oligonucleotide conjugated to a secondary capture molecule (antibody) that has bound a target molecule of interest on the loaded capture matrix. This advantageously results in a decrease of false positive amplicons. Furthermore, partition 2 is not necessary if the secondary capture molecules comprising the oligonucleotides are already added in partition 1. This allows a direct transfer of the loaded capture matrix comprising the bound secondary capture molecules comprising the attached oligonucleotides from partition 1 to partition 3.

A further embodiment is illustrated in FIG. 2. Partition 1 may here contain the loaded capture matrix with the bound analyte (see above for detailed description). The loaded capture matrix is then transferred to Partition 2. Such transfer can be performed as described elsewhere herein. The loaded capture matrix is contacted with a secondary capture molecule (in FIG. 1 an antibody) that binds the target analyte that is bound to the loaded capture matrix and to which an oligonucleotide is attached (partition 1 preferred, partition 2 optionally). The oligonucleotide comprising (i) the at least one nuclease target site that is complementary to the at least one nuclease target site of the barcode label of the detection molecule and (ii) the at least one analyte specific sequence that is complementary to the analyte specific sequence of the barcode label of the detection molecule is attached to the secondary capture molecule whereby it is associated with the loaded capture matrix. In the embodiment illustrated in FIG. 2, the oligonucleotide does not have blocked 3′OH end and is attached via a linker to the secondary capture molecule. However, the 3′ OH ends of the oligonucleotide and the barcode label may also be blocked. After binding, the loaded capture matrix is preferably washed to remove any unbound secondary capture molecules (partition 2).

Afterwards, the loaded capture matrix may be perfused with mixture of barcode labels that have a region specifically hybridizing with the oligonucleotides attached to the secondary capture molecules. The barcode labels essentially fulfil the same criteria as described for the embodiment illustrated in FIG. 1. However, in the embodiment illustrated in FIG. 2, the oligonucleotide attached to the secondary capture molecule and the barcode label comprise free 3′ OH ends. Furthermore, the barcode labels comprise a universal primer target site 5′ to the nuclease target site (NTS). After hybridization of the barcode labels to the oligonucleotides, the capture matrix may be washed one or more times to remove any unbound barcode labels. The hybridized barcode labels are released by using one or multiple endonucleases (preferred restriction endonucleases).

After cleavage of the hybrid, the cleaved barcode labels are collected and transferred to a partition 3 for subsequent amplification.

The barcode labels may be amplified using a PCR reaction or preferably, an isothermal amplification reaction. For amplification, the universal primer sites illustrated in FIG. 2 may be employed using any suitable primer or primer combination. In a further embodiment, the barcode sequences Bp and/or B_{T} are not directly present in the barcode label but are added during the amplification reaction, so that they are present in the sequenceable molecule.

The general procedure of the embodiment illustrated in FIG. 1 is further illustrated in FIG. 3. In A, a cell-laden matrix (1), which preferably is a hydrogel bead, and a capture matrix (2), which preferably is a hydrogel bead, are positioned in close proximity within a first partition, preferably an isolated compartment at a position X|Y of a device, which according to a preferred embodiment is a microfabricated cell culture device. The cell-laden matrix comprises in the illustrated embodiment a single cell (3), which secretes two biomolecules of interest (4a and 4b). However, the cell-laden matrix may also comprise two of more cells which secrete two or more biomolecules of interest or an empty matrix where one or more cells have been present (e.g. cell(s) may have undergone apoptosis or may escape from the matrix by cell migration) and have secreted one or more or two or more biomolecules of interest. The capture matrix (2) comprises in the illustrated embodiment two different types of capture molecules (5a and 5b), which specifically bind their target biomolecules of interest (4a and 4b). The different types of capture molecules are in the illustrated embodiment provided by antibodies with different specificities against the secreted biomolecules of interest. The capture matrix preferably comprises a plurality of capture molecules of the same type to ensure efficient capture of a target biomolecule of interest. The capture molecules may be provided in excess of the expected number of secreted biomolecules of interest.

In B, the cell-laden matrix (1) is incubated to allow sufficient secretion of the biomolecules of interest which diffuse from the cell-laden matrix (1) to the capture matrix (2), where a biomolecule of interest is bound by the matching type of capture molecule (see interaction pairs 4a/5a and 4b/5b). Unbound molecules may be washed away.

In C, one or more types of secondary capture molecules comprising attached, e.g. conjugated, oligonucleotides are added. In the illustrated embodiment, two types of secondary capture molecules are added (6a and 6b), wherein each type of secondary capture molecule specifically binds a target biomolecule of interest. Importantly, each type of secondary capture molecule comprises an attached oligonucleotide, which comprises an analyte specific sequence (ASS′) that is complementary to the analyte specific sequence (ASS) of the barcode label and which indicates the specificity of the secondary capture molecule for its target analyte and a nuclease target site (NTS′) that is complementary to the nuclease target site (NTS) of the barcode label. The oligonucleotide may be attached via a linker to the secondary capture molecule and preferably comprises a blocked 3′OH (see also FIG. 1). In one embodiment, the linker is provided by a photocleavable spacer. In the illustrated embodiment the different types of secondary capture molecules are provided by antibodies which bind the target biomolecule of interest at a different epitope than the capture molecule of the capture matrix that binds and thereby loads the biomolecules of interest to the capture matrix. Thereby, a complex is formed, comprising the capture molecule of the capture matrix, the biomolecule of interest and the secondary capture molecule with the attached oligonucleotide which thereby is associated with the capture matrix (see complex 4a/5a/6a/7a and 4b/5b/6b/7b).

In D, the loaded capture matrix comprising the bound secondary capture molecules and associated oligonucleotides is transferred from the first partition to a third partition, preferably an isolated compartment at a position X|Y of a device, which according to a preferred embodiment is a multi-well plate such as a 96 well plate. The transfer of the capture matrix from partition 1 to partition 3 may be performed using a RFCP geometry as described in WO 2019/048713 A1. The capture matrix is positioned within partition 3 in a manner that enables individual washing of the positioned capture matrix without affecting other capture matrices. Multiple capture matrices might be positioned within multiple partitions of the third partition. The removal of the capture matrix which comprises the complexes comprising the capture molecule and the biomolecule of interest from the first partition (see above) leaves the cell-laden matrix in the first partition. As illustrated in D, an “unloaded” further capture matrix may then be added/loaded into the first partition and a new cycle of loading and capturing may be performed at different time-points. The steps may be repeated at several time-points. To indicate the different time points, preferably different time points in the sequenceable product, a different barcode sequences B_{T} is provided at each different time point of analysis/capture, wherein the barcode sequences B_{T} preferably is either directly included in the barcode label or is introduced during the amplification reaction, so that it is present in the sequenceable molecule.

In E, according to one embodiment (see also FIG. 2) the capture matrix is transferred from the first partition to a second partition which preferably is an isolated compartment at a position X|Y of a device, which according to a preferred embodiment is a microfabricated cell culture device. In the second partition the complex comprising the capture molecule of the capture matrix, the biomolecule of interest, the secondary capture molecule and the oligonucleotide (see complex 4a/5a/6a/7a and 4b/5b/6b/7b) hybridizes to a specific barcode label, also referred to as target-identification and quantification oligonucleotide sequences (TIQOS) (8) which comprises at least (i) the barcode sequence B_{S}, and (ii) a barcode sequence (B_{T}) for indicating a time information, and/or (iii) a barcode sequence (B_{P}) for indicating position information of the cell-laden matrix, and (iv) a primer target sequence and (v) optionally a unique molecular identifier (UMI) sequence. The further elements of the barcode label (such as the nuclease target site (NTS) and the analyte specific sequence (ASS)) have been described above. According to one embodiment, the oligonucleotide is released from the secondary capture molecule in advance of the hybridization reaction to the barcode label, e.g. when a photocleavable linker is used to attach the oligonucleotide to the secondary capture molecule. Unbound barcode labels may be washed away.

In a preferred embodiment (see also FIG. 1), the capture matrix with said complex comprising the capture molecule, the biomolecule of interest, the secondary capture molecule and the oligonucleotide (see complex 4a/5a/6a/7a and 4b/5b/6b/7b) is directly transferred from a first partition into a third partition preferably an isolated compartment at a position X|Y of a device, which according to a preferred embodiment is a multi-well plate such as a 96 well plate. The transfer of the capture matrix from partition 1 to partition 3 may be performed using a RFCP geometry as described in WO 2019/048713 A1. After transfer the conjugated oligonucleotides (7) hybridize to the barcode labels (e.g. a target-identification and quantification oligonucleotide sequence (TIQOS)) (8) which comprises at least (i) the barcode sequence B_{S}, and (ii) a barcode sequence (B_{T}) for indicating a time information, and/or (iii) a barcode sequence (B_{P}) for indicating position information of the cell-laden matrix, and (iv) a primer target sequence and (v) optionally a unique molecular identifier (UMI) sequence. The further elements of the barcode label (such as the nuclease target site (NTS) and the analyte specific sequence (ASS)) have been described above. The barcode label preferably comprises a blocked 3′-OH to prevent elongation of the barcode label. As is described herein, the oligonucleotide may be released from the secondary capture molecule and thus the capture matrix complex in advance of the hybridization reaction to the barcode label (also referred to as TIQOS in FIG. 1), e.g. when a photocleavable linker is used to attach the oligonucleotide to the secondary capture molecule. Unbound barcode labels may be washed away.

In F, after hybridization of the oligonucleotide and the barcode label, a sequence-specific nuclease (11) binds to the formed nucleic-acid double strand at the formed cleavable recognition site. The binding of the sequence-specific nuclease and cleavage results in the release of a cleaved and thus degraded barcode label with a free 3′OH end (see also FIG. 1).

In G, in a preferred embodiment an extension oligonucleotide comprising a universal primer target sequence, a scrambled nuclease target site (NTS*) capable of hybridizing to the cleaved nuclease target site remaining in the cleaved barcode label, a universal adapter sequence capable of hybridizing to the complementary sequence in the barcode label and having a blocked 3′OH hybridizes to the cleaved barcode label which is then extended by an elongation reaction using the extension oligonucleotide (10) as a template (see also FIG. 1).

During the elongation reaction the universal primer sequence is incorporated into the extended barcode label. An amplifiable and furthermore already sequenceable reaction product is thereby generated which may comprise in one embodiment at least (i) the barcode sequence (B_{S}), and (ii) a barcode sequence (B_{T}) for indicating a time information, and/or (iii) a barcode sequence (B_{P}) for indicating position information of the cell-laden matrix, and (iv) a primer sequence and (v) optionally a unique molecular identifier (UMI) sequence. The generation of the sequenceable reaction product preferably comprises the use of at least one oligonucleotide, which in one embodiment is a universal primer, that is capable of hybridizing to the barcode label.

Preferably, G comprises performing an amplification reaction using a primer or primer combination. In the illustrated embodiment, such amplification reaction is performed after performing step F. The amplification reaction is indicated in FIG. 3 as (G) and comprises performing an amplification reaction with a primer or primer combination. If a single primer is used, a linear amplification can be performed by performing several amplification cycles. The use of a primer combination such as a primer pair allows to perform a PCR reaction. The primer or primer combination as well as the additional components required for performing the amplification reaction (such as a polymerase, dNTPs, buffers) may be added to the compartments (e.g. wells) of the third partition that comprise the transferred capture matrix, or may be provided in advance.

The amplification products may then be subsequently sequenced. As is described herein, the method according to the present invention provides multiple pooling options, allowing to make the sequencing very cost and time efficient.

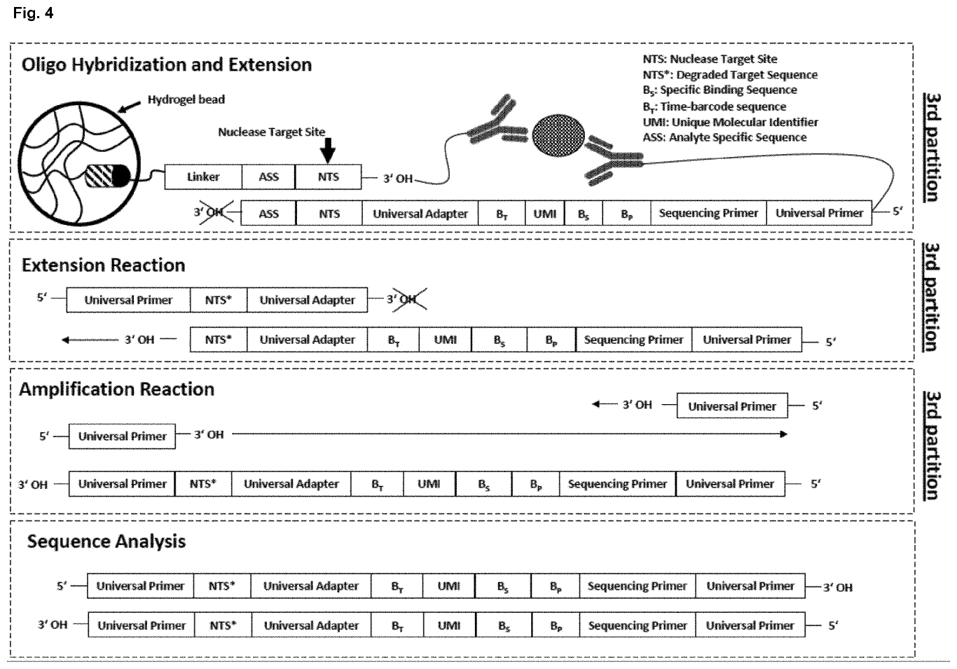

Further embodiments of this strategy employing endonuclease cleavage are illustrated in FIG. 4-FIG. 6, also referred to herein as proximity degradation assay.

A 1^{st} partition (e.g. provided in a microfluidic cell culture chip located within an incubation chamber), contains a capture matrix with at least one type of capture molecule (e.g. an antibody) having conjugated an oligonucleotide that contains an analyte specific sequence (ASS) and a bound analyte. The capture molecule is immobilized within capture matrix in a releasable manner. One end of the oligonucleotide (as shown preferably the 5′end) is attached to the capture matrix via a linker as described herein. The other end of the oligonucleotide (preferably the 3′ end) is conjugated to the capture molecule (e.g. antibody). Thus, the oligonucleotide does not contain a free 3′OH. In particular, the oligonucleotide (e.g. first proximity probe) is immobilized within a capture matrix that is a hydrogel matrix. The immobilization of the oligonucleotide/first proximity probe within a hydrogel matrix is advantageous as it enables the positioning of said hydrogel next to a (single) cell or multiple cells in a controlled manner e.g. by using microfluidic structures as well as the subsequent reduction of the reaction volume (partition 1). Thus, the analyte binding can occur within a reaction volume in the range of 100 pL to 200 nL thereby increasing sensitivity.

The capture matrix from partition 1 is directly transferred into a 3^{rd} partition (e.g. well of a microtiter plate or a reaction tube; (partition 2 is obsolete). Partition 3 contains a mixture of different types of detection molecules with different target analyte specificities. The detection molecules contain barcode labels having a blocked 3′ OH and indicating the target specificity, and optionally quantity, time and position information, and act as partner for binding the corresponding types of oligonucleotides that are (or were) conjugated to the capture molecules. The one of more types of capture molecules with the bound target analyte and attached oligonucleotide are preferably released from the capture matrix to diffuse into solution (optionally: the capture molecules are not released). Release can be achieved via degradation of the linker that attaches the oligonucleotide to the capture matrix. Within the solution, the one or more matching types of detection molecules are present. The detection molecules comprise a secondary capture molecule that binds to the target analyte. The binding of the detection molecule to the analyte that has bound already the capture molecule (e.g. an antibody) of the capture matrix to which the oligonucleotide is still attached results in an increased proximity between said oligonucleotide and the barcode label of the detection molecule, thereby increasing the likelihood of hybridization between the oligonucleotide and the barcode label (a similar principle is known from a conventional Proximity Ligation Assay (PLA) or Proximity Extension Assay (PEA)). The oligonucleotide bound to the capture molecule and the barcode label of the detection molecule hybridize via the complementary analyte specific sequence (ASS) and (ASS′), in the figures only referred to as ASS for the ease of simplicity. Thereby a double-stranded DNA hybrid is formed in the complementary portions, whereby at least one cleavable recognition site for an endonuclease is formed in the double-stranded portion by the nuclease target site (NTS) or the barcode label and the complementary sequence of the proximity oligonucleotide. The cleavable recognition site for an endonuclease is then acting as a target for (one or multiple) endonuclease(s), preferably restriction endonucleases. Degradation by the one or more endonucleases results in a free 3′ OH of the barcode label. This degradation/cleavage results in an inactivation of the proximity oligonucleotide. Thus, every capture molecule comprising the proximity oligonucleotide can only be detected once, thereby decreasing the number of false positive amplicons.

The use of an endonuclease significantly increases the sensitivity of the proximity assay as the melting temperature of the DNA double strand between the proximity oligonucleotide and the barcode label can be adjusted in relation to the temperature optimum of the used endonuclease and vice versa by using different endonuclease types. Thus, the temperature is adjusted in a manner that 1) a hybridization and subsequent degradation is very unlikely for detection molecules that are free in solution and that have not bound any analyte and 2) a hybridization and subsequent degradation is very likely for the oligonucleotide and barcode label that are in close proximity due to an analyte binding event. Endonuclease degradation results in cleaved barcode labels having free 3′OH ends that can thus be elongated in an extension reaction at the free 3′ OH end after hybridizing with an extension oligonucleotide located e.g. in partition 3. Said extension oligonucleotide preferably has a blocked 3′ OH. As is illustrated in FIG. 4, the amplifiable molecule is provided in the extension reaction by hybridizing an extension oligonucleotide to the cleaved and optionally further processed barcode label, wherein the extension oligonucleotide provides a template for the extension reaction. The generated amplifiable molecule preferably includes a universal primer target sequence. As is illustrated in FIG. 4, such universal primer target sequence may be added at the 3′ end of the barcode label, by hybridizing an extension oligonucleotide to the cleaved and optionally further processed barcode label, wherein the extension oligonucleotide provides the template for adding in the extension reaction the universal target primer sequence at the 3′ end of the barcode label (referred to as universal primer in FIG. 4). The extension reaction thus results in the addition of a primer sequence to the barcode label. Said extension reaction ensures that only barcode labels which have been degraded (i.e. cleaved barcode labels) are extended and not conjugated oligonucleotides or barcode labels. In addition, the extension reaction using said extension oligonucleotide enables to perform all different steps within one well/reaction volume without washing steps. The cleaved and extended barcode label is subsequently amplified with a primer or primer pair e.g. by using a PCR reaction or preferably by using an isothermal amplification. In summary, this embodiment in particular comprises the following critical steps:
1. Analyte Binding
2. Proximity induced hybridization
3. Degradation
4. Extension
5. Amplification

In a further embodiment, the barcode sequences Bp and/or B_{T} are not directly present in the barcode label but are added during the amplification reaction, so that they are present in the sequenceable molecule.

The described embodiment using a combination of (i) an proximity oligonucleotide that is attached to the capture matrix and the capture molecule of the capture matrix and (ii) the endonuclease cleavage has several important advantages:
There is no need for 2^{nd} microfluidic chip at pipetting robot reducing consumable costs.
A significant higher sensitivity can be achieved due to the PDA (Proximity Degradation Assay) assay (zeptomole range) which is advantageous and essential for detecting low secreted amounts from single cells.
Amplification reaction as well as PDA assay can be performed within the same well (no additional pipetting steps).
PDA assay also works with multiplexing, and thus, no sequential washing is required.
PDA assay significantly reduces cross reactivities, because mismatched molecules could not hybridize to a double stranded DNA as a target for the nuclease.
A decrease of false positive amplicons is achieved due to use of endonuclease and significantly increased specificity in comparison to conventional polymerases+combination of different endonucleases possible.
A conventional PLA assay always results in a linear amplification of non-ligated oligonucleotides. This results in a more complex analysis and decreases possible degree of multiplexing due to limited NGS reads. This is, however, not the case for the present invention, as the extension and subsequent amplification can only occur after a restriction event performed by an endonuclease (resulting in a free 3′OH group at the barcode label). Thus, no linear amplification of single oligonucleotides occurs, thereby resulting in higher multiplexing capabilities.

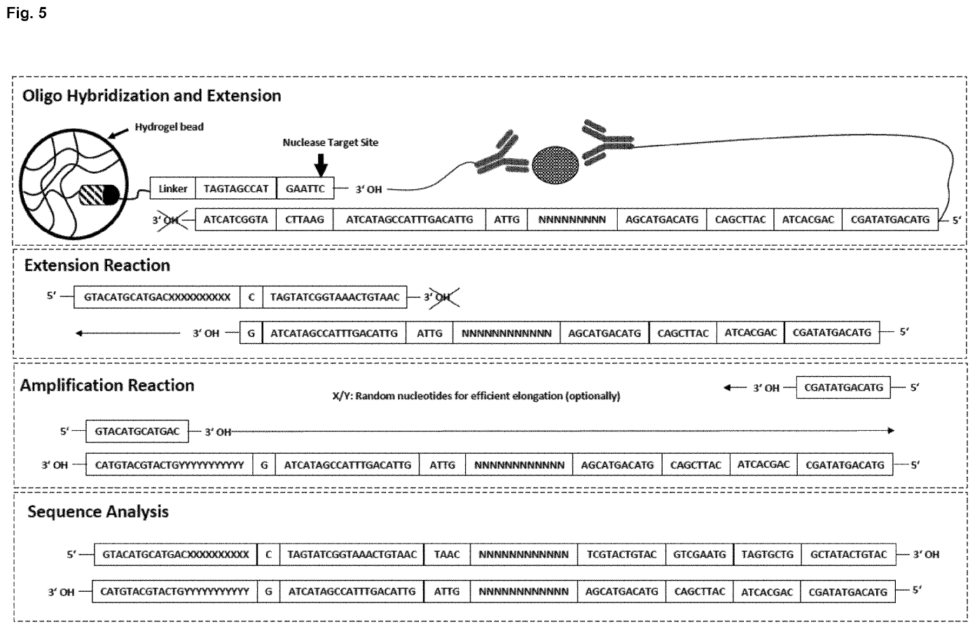

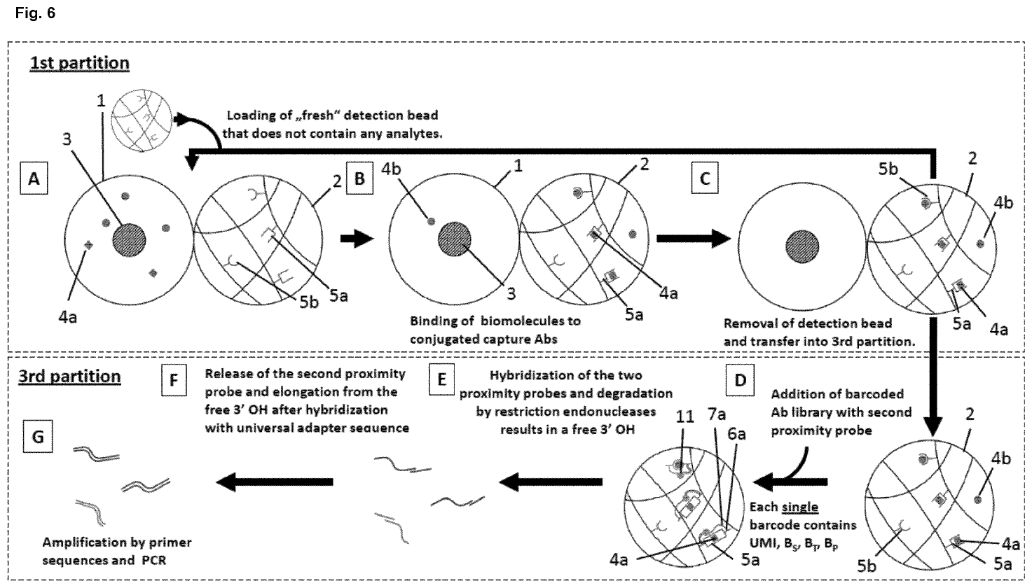

FIG. 5 illustrates a particular embodiment of FIG. 4, including an exemplified sequence of the above described oligonucleotide and barcode label.

The general procedure of the embodiment illustrated in FIG. 4 is further illustrated in FIG. 6. As illustrated in FIG. 6 A, a cell-laden matrix (1), which preferably is a hydrogel bead, and a capture matrix (2), which preferably is a hydrogel bead, are positioned in close proximity within a first partition, preferably an isolated compartment at a position X|Y of a device, which according to a preferred embodiment is a microfabricated cell culture device. The cell-laden matrix comprises in the illustrated embodiment a single cell (3), which secretes two biomolecules of interest (4a and 4b) or more cells which secrete two biomolecules of interest or an empty matrix where one or more cells have been present (e.g. cell(s) may have undergone apoptosis or may escape from the matrix by cell migration) and have secreted two biomolecules of interest. The capture matrix (2) comprises in the illustrated embodiment two different types of capture molecules (5a and 5b), which specifically bind the biomolecules of interest (4a and 4b). The capture molecules are conjugated to a proximity oligonucleotide as disclosed e.g. in FIG. 4 and described herein, which optionally comprises a degradable linker. One end of the proximity oligo is attached to the hydrogel via said linker, the other end is conjugated to the capture molecule resulting in a blocked 3′OH of the proximity oligonucleotide. The different types of capture molecules are in the illustrated embodiment provided by antibodies with different specificities against the secreted biomolecules of interest. The capture matrix preferably comprises a plurality of capture molecules of the same type to ensure efficient capture of a biomolecule of interest. The capture molecules may be provided in excess of the expected number of secreted biomolecules of interest. In B, the cell-laden matrix (1) is incubated to allow sufficient secretion of the biomolecules of interest which diffuse from the cell-laden matrix (1) to the capture matrix (2), where a biomolecule of interest is bound by the matching type of capture molecule (see interaction pairs 4a/5a and 4b/5b). Unbound molecules may be washed away.

In C, the capture matrix is transferred to a third partition, preferably an isolated compartment at a position X|Y of a device, which according to a preferred embodiment is a multi-well plate such as a 96 well plate. The transfer of the capture matrix from partition 1 to partition 3 may be performed using a RFCP geometry as described in WO 2019/048713 A1. The capture matrix is positioned within partition 3 in a manner that enables individual washing of the trapped capture matrix without affecting other capture matrices. Multiple capture matrices might be positioned within multiple partitions of the third partition. The removal of the capture matrix which comprises the complexes comprising the capture molecule and the biomolecule of interest from the first partition leaves the cell-laden matrix in the first partition. As illustrated in D, an “unloaded” capture matrix may be added/loaded into the first partition and a new cycle may be performed at different time-points. The steps may be repeated at several time-points.

After transfer of the capture matrix with said complex comprising the capture molecule and the biomolecule of interest (see complex 4a/5a and 4b/5b), one or more types of detection molecules are added, here two types of detection molecules (6a and 6b), wherein each type of detection molecule specifically binds a biomolecule of interest are added. Importantly, each type of detection molecule comprises a barcode label as is illustrated in FIG. 4 and described herein. The barcode label may be provided by an oligonucleotide sequence that may be attached via a linker to the secondary capture molecule of the detection molecule. The barcode label comprises a blocked 3′-OH to prevent elongation. In an embodiment, the linker is provided by a photocleavable spacer. In the illustrated embodiment the specificity of the different types of detection molecules are provided by antibodies which bind their target biomolecule of interest at a different epitope than the capture molecules (antibodies) of the capture matrix. Thereby, a complex is formed, comprising the oligonucleotide (e.g. first proximity probe), the capture molecule of the capture matrix, the biomolecule of interest and the detection molecule comprising the secondary capture molecule bound to the target biomolecule of interest and the barcode label (e.g. second proximity probe; see complex 4a/5a/6a and 4b/5b/6b). Within this complex, the oligonucleotide and barcode label can build a nucleotide double-strand at the complementary analyte-specific sequence (ASS) and/or (NTS).

In D, after hybridization of the oligonucleotide and the barcode label, a sequence-specific nuclease binds to the formed oligonucleotide double-strand at the formed cleavable recognition site (NTS). The binding of the sequence-specific nuclease and cleavage results in the release of a cleaved and thus degraded barcode label with a free 3′OH end (see also FIG. 4). After degradation an extension oligonucleotide comprising a universal primer sequence, a scrambled nuclease target site (NTS*) capable of binding to the cleaved nuclease target site of the barcode label, a universal adapter sequence binds to the cleaved barcode label (8). The extension oligonucleotide preferably comprises a blocked 3′OH. After binding of the extension oligonucleotide, the cleaved barcode label is extended using the extension oligonucleotide as a template. During the extension reaction the universal primer sequence of the extension oligonucleotide is incorporated into the thereby extended, cleaved barcode label.

In E, a sequenceable reaction product is generated which comprises in one embodiment at least (i) the barcode sequence (B_{S}), and (ii) a barcode sequence (B_{T}) for indicating a time information, and/or (iii) a barcode sequence (B_{P}) for indicating position information of the cell-laden matrix, and (iv) a universal primer sequence, and (v) optionally a unique molecular identifier (UMI) sequence. The generation of the sequenceable reaction product comprises the use of at least one oligonucleotide, which in one embodiment is a universal primer, that is capable of hybridizing to the barcode label.

Preferably, G comprises performing an amplification reaction using a primer or primer combination. In the illustrated embodiment, such amplification reaction is performed after performing step F. The amplification reaction is indicated as step G and comprises performing an amplification reaction with a primer or primer combination. If a single primer is used, a linear amplification can be performed by performing several amplification cycles. The use of a primer combination such as a primer pair allows to perform a PCR reaction. The primer or primer combination as well as the additional components required for performing the amplification reaction (such as a polymerase, dNTPs, buffers) may be added to the compartments (e.g. wells) of the third partition that comprise the transferred capture matrix, or may be provided in advance.

As is described herein, the barcode label may be released from the detection molecule in advance of the amplification reaction, e.g. when a photocleavable linker is used.

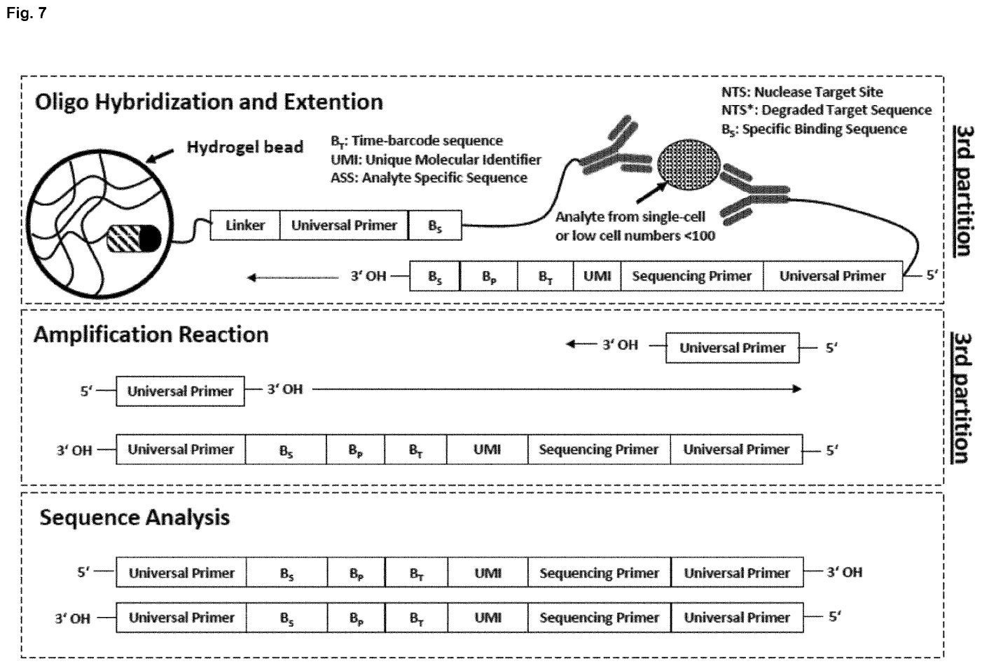

A further strategy described herein is also based on the proximity extension assay, however, without using an endonuclease for cleavage of the barcode label (see FIG. 7).

A 1^{st} partition (e.g. provided in a microfluidic cell culture chip located within an incubation chamber), contains a capture matrix with antibody capture molecule having an oligonucleotide conjugate that is later used for performing a proximity extension assay (PEA) and bound analyte. One end of the oligonucleotide is attached to the capture matrix (e.g. hydrogel) via a linker. The other end is conjugated to the capture molecule. Thus, no free 3′OH is present in the oligonucleotide. Optionally, the free 3′OH is blocked independently from the incorporation within the capture matrix (e.g. hydrogel). In particular, the oligonucleotide is immobilized within a capture matrix (e.g. hydrogel matrix) e.g. by Tamavidin/Biotin linkage. The immobilization of the oligonucleotide within a capture matrix is advantageous as it enables the positioning of said capture matrix next to a (single) cell or multiple cells in a controlled manner e.g. by using microfluidic structures as well as the subsequent reduction of the reaction volume (partition 1). Thus, the analyte binding can occur within a reaction volume in the range of 100 pL to 200 nL thereby increasing sensitivity. The capture molecule is immobilized within the capture matrix in a releasable manner. In terms of conventional PLA/PEA assay the two proximity probes (here oligonucleotide and barcode label of the detection molecule) comprise or consist of conjugated antibodies that bind different epitopes on the same analyte are simultaneously transferred into a partition in which an analyte is present. Thus, the proximity probes are present in a similar concentration and initial analyte binding occurs in the presence of both proximity probes. With regard to the current disclosure, the initial binding of an analyte to an oligonucleotide (e.g. first proximity probe) is performed in a first partition (partition 1). Afterwards, the complex comprising or consisting of the oligonucleotide and bound analyte is transferred into another partition (partition 3)

In a 3^{rd} partition (e.g. well of a microtiter plate or a reaction tube) the detection molecule comprising the barcode label is present. The transfer of the capture matrix into a third partition is advantageous as it enables the addition of the detection molecule decoupled from the cell cultivation area. Capture matrix from partition 1 is directly transferred into partition 3 (partition 2 is obsolete). Partition 3 contains a mixture of one or more types of detection molecules (e.g. conjugated secondary antibodies with different specificities). The detection molecules comprise conjugated barcode labels containing specificity, time and position information and act as partner for binding to corresponding oligonucleotide conjugated to capture molecule. The oligonucleotide of the capture matrix comprises a specificity sequence that is complementary or the specificity sequence of the barcode label allowing for hybridization. After hybridization of the oligonucleotide and the barcode label (e.g. of the two conjugated antibodies), the barcode label conjugated to the second capture molecule (e.g. an antibody) is extended, wherein the oligonucleotide of the capture matrix represents a template for transferring sequence information into the barcode label. In particular, during the extension reaction, a common universal primer sequence is incorporated into the barcode label. Optionally: capture molecules with bound analytes are released from capture matrix to diffuse into solution. Within solution the detection molecule, particularly the secondary capture molecule of the detection molecule, binds to analyte and couples by hybridization (PEA assay). Polymerase extension reaction then adds a common primer sequence to the barcode sequence of the detection molecule. Afterwards, the extended oligonucleotide is amplified within the same well using a PCR or isothermal amplification reaction.

This strategy has the following advantages:
The likelihood of correct hybridization event (=hybridization of the barcode label of the detection molecule with an proximity oligonucleotide of the capture molecule, whereas both have bound the same analyte molecule) is significantly increased as the analyte is bound already to the capture matrix before adding the detection molecule which results in a decrease of false positive hybridization events.
The pre-binding ensures that only analytes of interest are presented to the detection molecules reducing cross-reactivities.
No need for 2^{nd} microfluidic chip at pipetting robot reducing consumable costs
Significant higher sensitivity due to/PEA assay (zeptomole range) which is essential for detecting low secreted amounts from single cells
Amplification reaction as well as PEA assay can be performed within the same well (no additional pipetting steps)
PLA assay also works with multiplexing->no sequential washing required

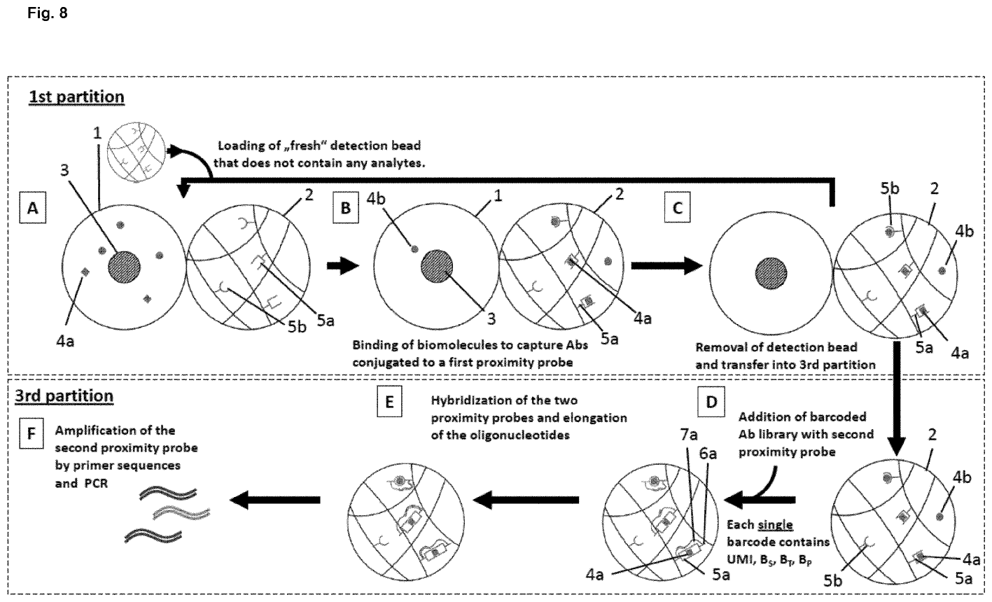

The general procedure of the embodiment illustrated in FIG. 7 is further illustrated in FIG. 8. As illustrated in FIG. 8 A, a cell-laden matrix (1), which preferably is a hydrogel bead, and a capture matrix (2), which preferably is a hydrogel bead, are positioned in close proximity within a first partition, preferably an isolated compartment at a position X|Y of a device, which according to a preferred embodiment is a microfabricated cell culture device. The cell-laden matrix comprises in the illustrated embodiment a single cell (3), which secretes two biomolecules of interest (4a and 4b) or more cells which secrete two biomolecules of interest or an empty matrix where one or more cells have been present (e.g. cell(s) may have undergone apoptosis or may escape from the matrix by cell migration) and have secreted two biomolecules of interest. The capture matrix (2) comprises in the illustrated embodiment two different types of capture molecules (5a and 5b), which specifically bind the biomolecules of interest (4a and 4b). The capture molecules are conjugated to an oligonucleotide which contains a specificity barcode B_{S} a universal primer sequence and optionally a degradable linker. One end of the oligonucleotide is attached to the capture matrix (e.g. hybrigel) via said linker, the other end is conjugated to the capture molecule resulting in a blocked 3′OH of the oligonucleotide. The different types of capture molecules are in the illustrated embodiment provided by antibodies with different specificities against the secreted biomolecules of interest. The capture matrix preferably comprises a plurality of capture molecules of the same type to ensure efficient capture of a biomolecule of interest. The capture molecules may be provided in excess of the expected number of secreted biomolecules of interest.

In B, the cell-laden matrix (1) is incubated to allow sufficient secretion of the biomolecules of interest which diffuse from the cell-laden matrix (1) to the capture matrix (2), where a biomolecule of interest is bound by the matching type of capture molecule (see interaction pairs 4a/5a and 4b/5b). Unbound molecules may be washed away.

In C, the capture matrix is transferred to a third partition, preferably an isolated compartment at a position X|Y of a device, which according to a preferred embodiment is a multi-well plate such as a 96 well plate. The transfer of the capture matrix from partition 1 to partition 3 may be performed using a RFCP geometry as described in WO 2019/048713 A1. The capture matrix is positioned within partition 3 in a manner that enables individual washing of the trapped capture matrix without affecting other capture matrices. Multiple capture matrices might be positioned within multiple partitions of the third partition. The removal of the capture matrix which comprises the complexes comprising the capture molecule and the biomolecule of interest from the first partition leaves the cell-laden matrix in the first partition. As illustrated in D, an “unloaded” capture matrix may be added/loaded into the first partition and a new cycle may be performed at different time-points. The steps may be repeated at several time-points.

In D, one or more types of detection molecules are added, here two types of detection molecules (6a and 6b), wherein each type of detection molecule specifically binds a biomolecule of interest. Importantly, each type of detection molecule comprises a barcode label which comprises at least (i) the barcode sequence (B_{S}), and (ii) a barcode sequence (B_{T}) for indicating a time information, and/or (iii) a barcode sequence (B_{P}) for indicating position information of the cell-laden matrix, and (iv) a primer sequence, and (v) optionally a unique molecular identifier (UMI) sequence. Thus, the specificity of the capture molecule can be determined based on the barcode label comprised in the detection molecule. The barcode label may be provided by an oligonucleotide sequence that may be attached via a linker to the secondary capture molecule, which together form the detection molecule. In an embodiment, the linker is provided by a photocleavable spacer. In the illustrated embodiment, the different types of detection molecules are provided by antibodies which bind the biomolecule of interest at a different epitope than the antibodies used for capturing. Thereby, a complex is formed, comprising the capture molecule, the oligonucleotide, the biomolecule of interest and the detection molecule comprising the barcode label (see complex 4a/5a/6a and 4b/5b/6b).

In E, after hybridization of the oligonucleotide and the barcode label at the complementary sequences (B_{S}), the barcode label is extended using the oligonucleotide as a template. During the extension reaction the universal primer sequence of the oligonucleotide is incorporated into the extended barcode label.

In F, a sequenceable reaction product is generated by amplification which comprises at least (i) the barcode sequence (B_{S}), and (ii) a barcode sequence (B_{T}) for indicating a time information, and/or (iii) a barcode sequence (B_{P}) for indicating position information of the cell-laden matrix, and (iv) a universal primer sequence, and (v) optionally a unique molecular identifier (UMI) sequence. The generation of the sequenceable reaction product comprises the use of at least one oligonucleotide, which in one embodiment is a universal primer, that is capable of hybridizing to the extended barcode label of the at least one type of detection molecule.

Preferably, G comprises performing an amplification reaction using a primer or primer combination. In the illustrated embodiment, such amplification reaction is performed after performing step F. The amplification reaction is indicated as step G and comprises performing an amplification reaction with a primer or primer combination. If a single primer is used, a linear amplification can be performed by performing several amplification cycles. The use of a primer combination such as a primer pair allows to perform a PCR reaction. The primer or primer combination as well as the additional components required for performing the amplification reaction (such as a polymerase, dNTPs, buffers) may be added to the compartments (e.g. wells) of the third partition that comprise the transferred capture matrix, or may be provided in advance.

As is described herein, the barcode label and/or the oligonucleotide may be released from their capture molecules in advance of the amplification reaction, e.g. when a photocleavable linker is used.

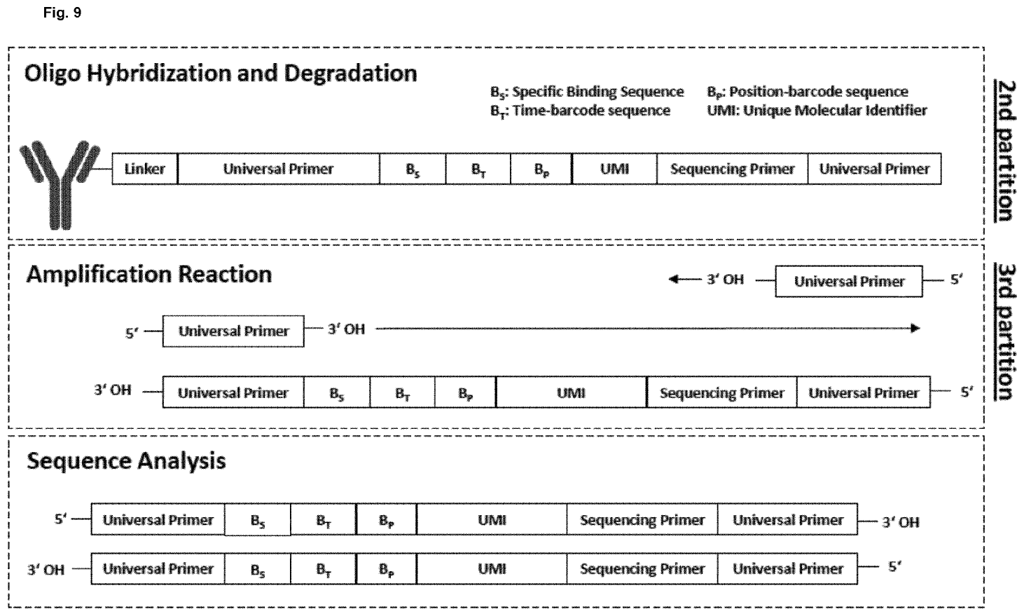

A further strategy is illustrated in FIGS. 9 to 12 which is based on the use of a single oligonucleotide/barcode label. FIG. 9 shows one embodiment, wherein detection molecule comprises a barcode label attached to a capture molecule, here an antibody, that is capable of specifically binding the target analyte of interest that is loaded onto the capture matrix. The different possible and preferred elements of the barcode label are shown in this embodiment. Preferably, the barcode label comprises all these elements. After binding to the loaded capture matrix, an amplification reaction can be performed followed by a sequencing reaction.

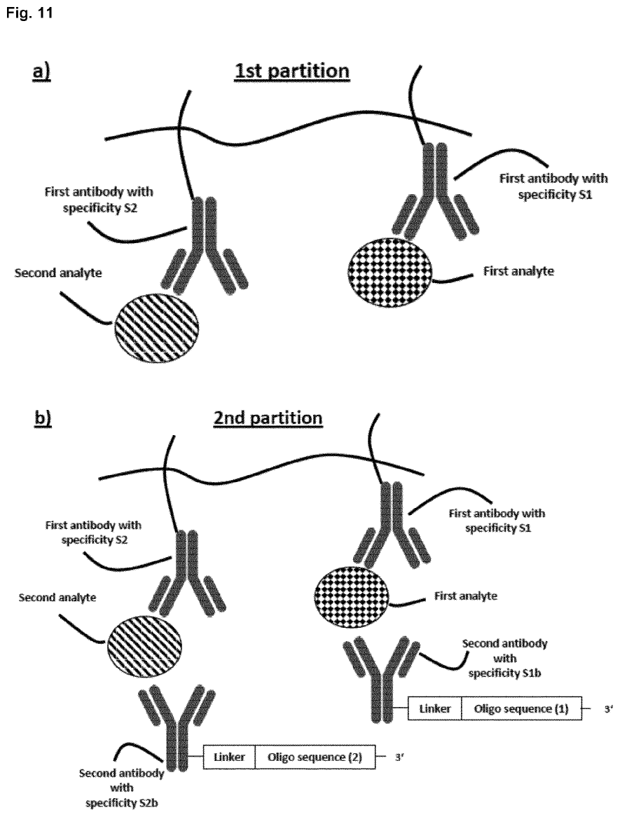

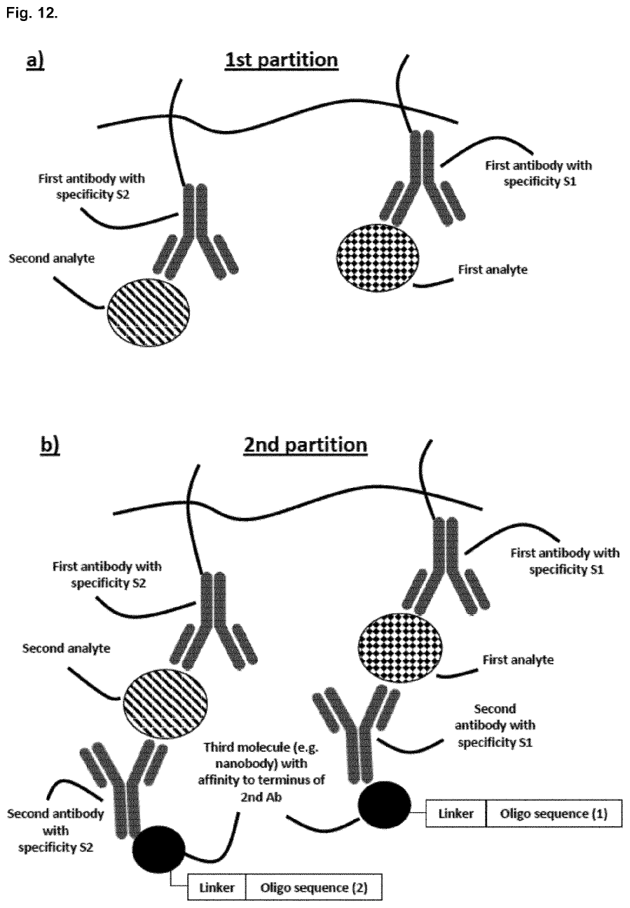

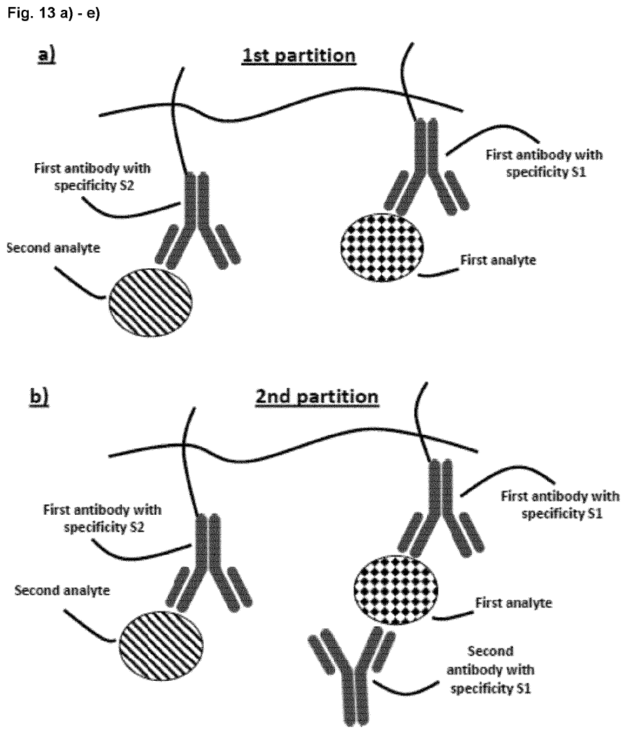

According to one embodiment illustrated in FIG. 11, the 1^{st} partition comprises the loaded capture matrix. The loaded capture matrix may then be transferred to a second partition for binding of the detection molecules comprising the barcode labels comprising the target specific barcode sequence B_{S} (in FIG. 11, the barcode label abbreviated as oligo sequence (1) for the first target analyte and as oligo sequence (2) for the second analyte). As is also illustrated in FIG. 9, the detection molecule preferably comprises a secondary antibody binding the target analyte conjugated to the barcode label. After binding, the capture matrix is preferably washed to remove any unbound detection molecules. The loaded capture matrix comprising the bound detection molecules may then be transferred to partition 3 (amplification reaction). The barcode label comprising the different barcode sequences may then be amplified, preferably using one or more universal primer target sites present in the barcode label. Amplification in partition 3 may be performed using a PCR reaction or an isothermal amplification reaction.

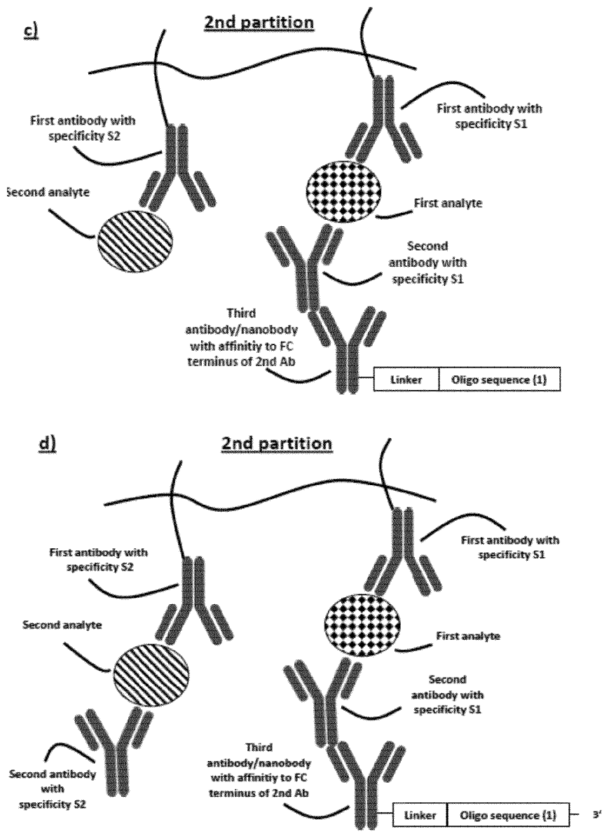

According to one embodiment illustrated in FIG. 12, the 1^{st} partition comprises the loaded capture matrix. The loaded capture matrix may then be transferred to a 2nd partition. In the 2^{nd} partition, the loaded capture matrix is contacted with one or more types of tagging molecules, wherein each type of tagging molecule is capable of binding to a different target analyte. Each type of tagging molecule comprises a binding molecule for binding its target analyte and an interaction moiety for binding to the detection molecule. In the embodiment illustrated in FIG. 12, the tagging molecules comprise an antibody (binding molecule) which is coupled to biotin or a biotin binding polypeptide (such as tamavidin). Each type of tagging molecule is bound to its corresponding type of detection molecule comprising a barcode label with a common barcode sequence B_{S} indicative for the target analyte. To allow binding, the detection molecule comprises a binding moiety that recognizes and binds the interaction moiety of the tagging molecule. The tagging molecule and the detection molecule are thus bound to each other via the interaction pair of the interaction moiety of the tagging moiety and the corresponding binding moiety of the detection molecule. A type of tagging molecule capable of binding a specific target analyte is bound to its corresponding type of detection molecule comprising a barcode label comprising a barcode sequence B_{S} indicative for the target analyte prior to contacting the so formed complex with the loaded capture matrix. Therefore, according to one embodiment, the loaded capture matrix is contacted with a mixture of secondary antibodies that bind to bound analytes as tagging molecules, wherein. said secondary antibodies have coupled tamavidin bound to a biotinylated oligonucleotide as detection molecule, the barcode label oligonucleotide containing specificity, time and position information. Afterwards the capture matrix is washed to remove any unbound tagging molecules, here secondary antibodies. The capture matrix containing the capture molecules, the bound target analyte and the bound tagging molecules conjugated to the detection molecules is transferred to partition 3 (amplification reaction).

In partition 3 the barcode labels transferred from partition 2 are amplified, e.g. using a PCR reaction or preferably an isothermal amplification reaction.

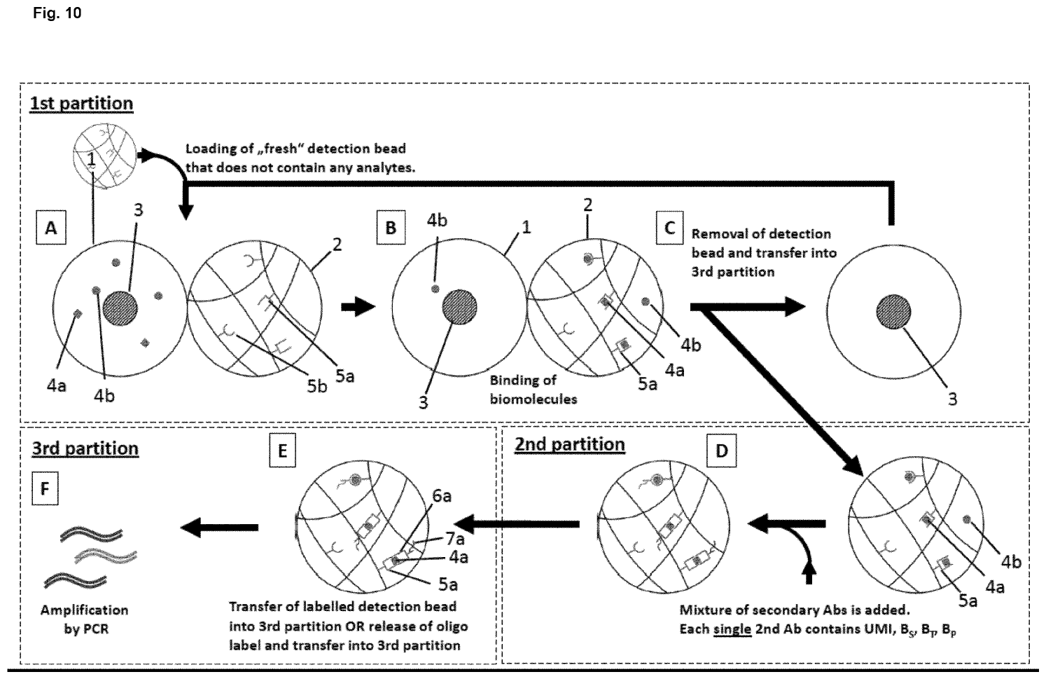

This method is further illustrated in FIG. 10.

As illustrated in A, a cell-laden matrix (1), which preferably is a hydrogel bead, and a capture matrix (2), which preferably is a hydrogel bead, are positioned in close proximity within a first partition, preferably an isolated compartment at a position X|Y of a device, which according to a preferred embodiment is a microfabricated cell culture device. The cell-laden matrix comprises in the illustrated embodiment a single cell (3), which secretes two biomolecules of interest (4a and 4b) or more cells which secrete two biomolecules of interest or an empty matrix where one or more cells have been present (e.g. cell(s) may have undergone apoptosis or may escape from the matrix by cell migration) and have secreted two biomolecules of interest. The capture matrix (2) comprises in the illustrated embodiment two different types of capture molecules (5a and 5b), which specifically bind the biomolecules of interest (4a and 4b). The different types of capture molecules are in the illustrated embodiment provided by antibodies with different specificities against the secreted biomolecules of interest. The capture matrix preferably comprises a plurality of capture molecules of the same type to ensure efficient capture of a biomolecule of interest. The capture molecules may be provided in excess of the expected number of secreted biomolecules of interest.

In B, the cell-laden matrix (1) is incubated to allow sufficient secretion of the biomolecules of interest which diffuse from the cell-laden matrix (1) to the capture matrix (2), where a biomolecule of interest is bound by the matching type of capture molecule (see interaction pairs 4a/5a and 4b/5b). Unbound molecules may be washed away.

In C, the capture matrix is transferred to a second partition, preferably an isolated compartment at a position X|Y of a device, which according to a preferred embodiment is a microfabricated device. The transfer of the capture matrix from partition 1 to partition 2 may be performed using a RFCP geometry as described in WO 2019/048713 A1. The capture matrix is trapped within partition 2 in a manner that enables individual perfusion and washing of the trapped capture matrix without affecting other capture matrices. Multiple capture matrices might be trapped within multiple partitions of the second partition. The removal of the capture matrix which comprises the complexes comprising the capture molecule and the biomolecule of interest from the first partition leaves the cell-laden matrix in the first partition. As illustrated in D, an “unloaded” capture matrix may be added/loaded into the first partition and a new cycle may be performed at different time-points. The steps may be repeated at several time-points.

In D, after transfer of the capture matrix, one or more types of detection molecules are added preferably by perfusing the capture matrix within the second partition. The detection molecules specifically bind biomolecules of interest that are immobilized within the capture matrix by a capture molecule (e.g. 5a). Afterwards, capture matrices are washed to remove any unbound detection molecules. In the illustrated embodiment the detection molecules are provided by an antibody which binds the biomolecule of interest at a different epitope than the antibody used for capturing. Thereby, a complex is formed, comprising the capture molecule, the biomolecule of interest and the detection molecule (see complex 4a/5a/6a and 4b/5b/6b).

Importantly, each type of detection molecule comprises a barcode label which comprises at least (i) the barcode sequence (B_{S}) and (ii) optionally a unique molecular identifier (UMI) sequence, and preferably in addition (iii) a barcode sequence (B_{T}) for indicating a time information, (iv) a barcode sequence (B_{P}) for indicating position information of the cell-laden matrix, and (v) a primer sequence. The barcode label may be provided by an oligonucleotide sequence that may be attached via a linker to the detection molecule. In an embodiment, the linker is provided by a photocleavable spacer.

In E, the capture matrix is transferred to a third partition, preferably an isolated compartment at a position X|Y of a device, which according to a preferred embodiment is a multi-well plate such as a 96 well plate. The transfer of the capture matrix from partition 2 to partition 3 may be performed using a RFCP geometry as described in WO 2019/048713 A1. The capture matrix is trapped within partition 2 in a manner that enables individual perfusion and washing of the trapped capture matrix without affecting other capture matrices. Multiple capture matrices might be trapped within multiple partitions of the second partition

In F, a sequenceable reaction product is generated which comprises at least (i) the analyte-specific sequence (ASS), and (ii) a barcode sequence (B_{T}) for indicating a time information, and/or (iii) a barcode sequence (B_{P}) for indicating position information of the cell-laden matrix, and (iv) a primer sequence and (v) optionally a unique molecular identifier (UMI) sequence. The generation of the sequenceable reaction product comprises the use of at least one oligonucleotide, which in one embodiment is a universal primer, that is capable of hybridizing to the barcode label of the at least one type of detection molecule.

Preferably, G comprises performing an amplification reaction using a primer or primer combination. In the illustrated embodiment, such amplification reaction is performed after performing step F. The amplification reaction is indicated as step G and comprises performing an amplification reaction with a primer or primer combination. If a single primer is used, a linear amplification can be performed by performing several amplification cycles. The use of a primer combination such as a primer pair allows to perform a PCR reaction. The primer or primer combination as well as the additional components required for performing the amplification reaction (such as a polymerase, dNTPs, buffers) may be added to the compartments (e.g. wells) of the third partition that comprise the transferred capture matrix, or may be provided in advance.

As is described herein, the barcode label may be released from the detection molecule in advance of the amplification reaction, e.g. when a photocleavable linker is used. The amplification products may then be subsequently sequenced. As is described herein, the method according to the present invention provides multiple pooling options, allowing to make the sequencing very cost and time efficient.

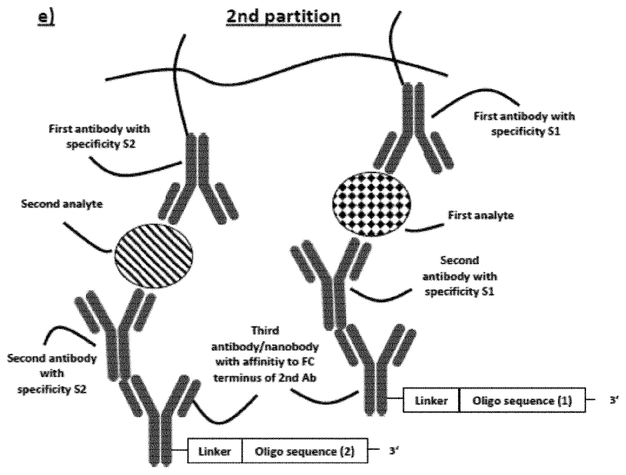

A further strategy is illustrated in FIGS. 13 to 16 which is based on the use of a detection molecule comprising a barcode sequence, wherein the method comprises the steps of (aa) adding a first type of tagging molecule, that specifically binds the first target biomolecule of interest that is captured by the capture molecule of the capture matrix to the capture matrix, thereby forming a complex comprising the loaded capture matrix and the tagging molecule bound to its target biomolecule of interest, and (bb) adding the first type of detection molecule wherein the first type of detection molecule comprises the barcode label and a binding moiety for binding the first type of tagging molecule and binding the first type of detection molecule to the tagging molecule.

A strategy described herein based on the use of a detection molecule in a method comprising steps (aa) and (bb) described above is illustrated in FIG. 13. In particular, the interaction moiety of the tagging molecule and the binding moiety of the detection molecule that binds said interaction moiety form part of an interaction pair, wherein said interaction pair is preferably selected from: the Fc region of an antibody molecule and an Fc region binding protein, wherein the Fc region binding protein is optionally selected from an anti-Fc antibody, an anti-Fc binding antibody fragment and a nanobody

A 1^{st} partition (e.g. provided in a microfluidic cell culture chip located within an incubation chamber), contains a cell laden matrix, a capture matrix (e.g. a capture bead) comprising one or more types of capture molecules and the released one or more target analyte(s) (biomolecule of interest). The capture molecule of the capture matrix may be provided by an antibody (as is illustrated in FIG. 13a). The cell-laden matrix may comprise one or more cells or the cell-laden matrix comprised at least one cell that has released one or more analytes of interest (an empty matrix where one or more cells have been present—e.g. cell(s) may have undergone apoptosis or may escape from the matrix by cell migration).

Capture matrix from partition 1 with bound analytes is transferred a 2^{nd} Partition (e.g. in a microfluidic processing chip). Transfer of the capture matrix from partition 1 to partition 2 may be performed using a RFCP geometry as described in WO 2019/048713 A1. The capture matrix is trapped within partition 2 in a manner that enables individual perfusion and washing of trapped capture matrix without affecting other capture matrices. Multiple capture matrices might be trapped within multiple partitions 2. The capture matrix is perfused with a first type of tagging molecule, that specifically binds the first target biomolecule of interest that is captured by the capture molecule of the capture matrix to the capture matrix, thereby forming a complex comprising the loaded capture matrix and the tagging molecule bound to its target biomolecule of interest (FIG. 13b). Afterwards, the capture matrix is washed to remove any unbound first type of tagging molecule. The capture matrix is then perfused with the first type of detection molecule wherein the first type of detection molecule comprises the barcode label and a binding moiety for binding the first type of tagging molecule and binding the first type of detection molecule to the tagging molecule. Preferably the first type of detection molecule is a conjugated antibody that binds to the first type of tagging molecule, preferably the FC region of an antibody bound to the biomolecule of interest (FIG. 13c). Afterwards, the capture matrix is washed again to remove any unbound first type of detection molecule. The barcode label provided by the first type of detection molecule (e.g. conjugated to the antibody) contains a barcode sequence for the specificity of the previously perfused secondary antibody, the barcode sequence representing the time at which the capture matrix has been transferred from partition 1 to partition 2, a barcode sequence for identifying the position within partition 1 from which the capture matrix has been transferred and an UMI sequence for absolute quantification using NGS. The process is repeated with different types of tagging molecules and different types of detection molecules (e.g. a second type, a third type, a fourth type and so on), thereby enabling the sequential labelling of bound analytes of different type located within a capture matrix (multiplexing) (FIG. 13d-e).

The capture matrix containing capture molecules (e.g. antibodies), analyte, bound tagging molecules and bound detection molecules is transferred to a 3^{rd} partition (e.g. well of a microtiter plate or a reaction tube), in particular for performing an amplification reaction. Barcode-labels of the capture matrices from partition 2 are amplified using a PCR reaction or preferred an isothermal amplification reaction.

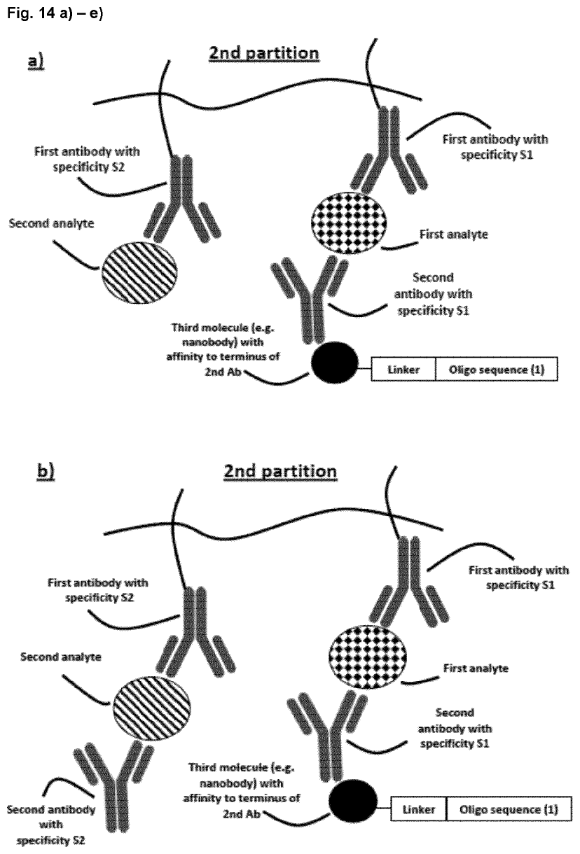

A further strategy described herein is also based on the use of a detection molecule in a method comprising steps (aa) and (bb) described above, however, the interaction moiety of the tagging molecule and the binding moiety of the detection molecule that binds said interaction moiety form part of an interaction pair, wherein said interaction pair is preferably selected from: biotin and a biotin binding polypeptide, wherein the biotin binding polypeptide is optionally selected from tamavidin, streptavidin and avidin (see FIG. 14).

A 1^{st} partition (e.g. provided in a microfluidic cell culture chip located within an incubation chamber), contains a cell laden matrix, a capture matrix (e.g. a capture bead) comprising one or more types of capture molecules and the released one or more target analyte(s) (biomolecule of interest). The capture molecule of the capture matrix may be provided by an antibody (as is illustrated in FIG. 13a). The cell-laden matrix may comprise one or more cells or the cell-laden matrix comprised at least one cell that has released one or more analytes of interest (an empty matrix where one or more cells have been present—e.g. cell(s) may have undergone apoptosis or may escape from the matrix by cell migration).

The capture matrix containing capture molecules and bound analytes is perfused with the first type of tagging molecule. This step can be performed in a 2^{nd} Partition (e.g. in a microfluidic processing chip). Such tagging molecule may be an antibody/nanobody comprising a biotin binding polypeptide, in particular tamavidin conjugated thereto (FIG. 14b). Then, the first type of detection molecule is perfused, wherein the detection molecule in particular comprises or consists of a biotinylated barcode label containing specificity, time, quantity and position information. Afterwards, the capture matrix is washed to remove any unbound tagging molecule and detection molecule. The process is preferably repeated with tagging molecules and detection molecules of different types (e.g. second type, third type, fourth type, etc.), preferably wherein each tagging molecule type binds to a different biomolecule of interest and wherein each detection molecule comprises a barcode label having a barcode sequence indicating the specificity of the tagging molecule (FIG. 14d-e). The capture matrix containing capture molecules, analyte, bound tagging molecule and bound detection molecule, wherein in particular tagging molecule and detection molecule are bound to each other via a biotin biotin-binding protein interaction, is transferred to partition 3 (amplification reaction).

As above, the capture matrix comprising the bound barcode label is transferred to a 3^{rd} partition (e.g. well of a microtiter plate or a reaction tube). In the 3^{rd} partition barcode labels of the capture matrix from partition 2 are amplified using a PCR reaction or preferred an isothermal amplification reaction.

This strategy is associated with the following advantages:
Use of commercially available ELISA pairs without requiring further conjugation of oligonucleotides and subsequent purification e.g. of conjugated antibodies.
The detection molecule is the same for all tagging molecules and only differs in terms of the type of conjugated barcode label (e.g. barcode sequence) reducing costs and quality control efforts.
All tagging molecules ca be labelled with the same strategy, e.g. biotinylated oligonucleotide, thereby reducing cost and production complexity.
The use of the biotin/tamavidin system results in a cost reduction in comparison to using an interaction pair based on an FC binding protein (e.g. antibody).

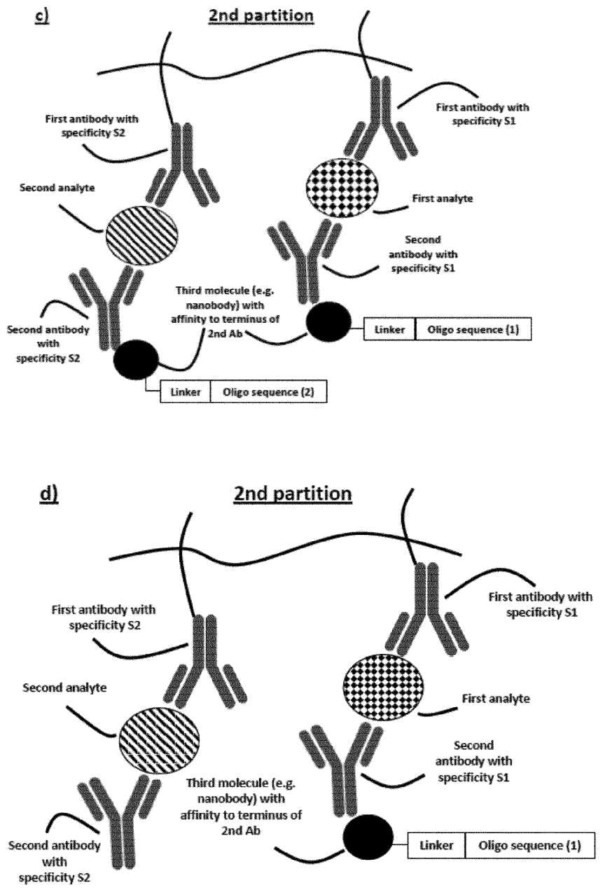

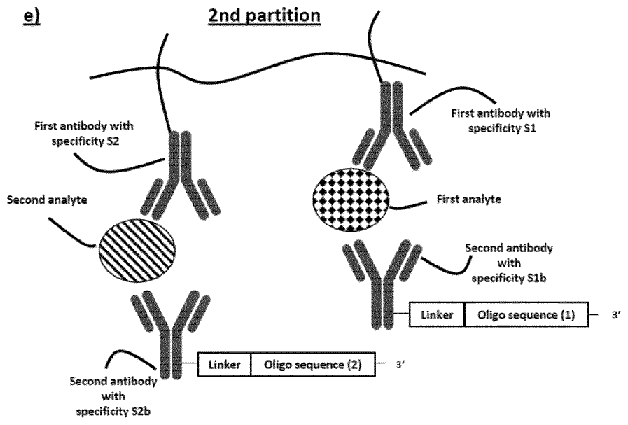

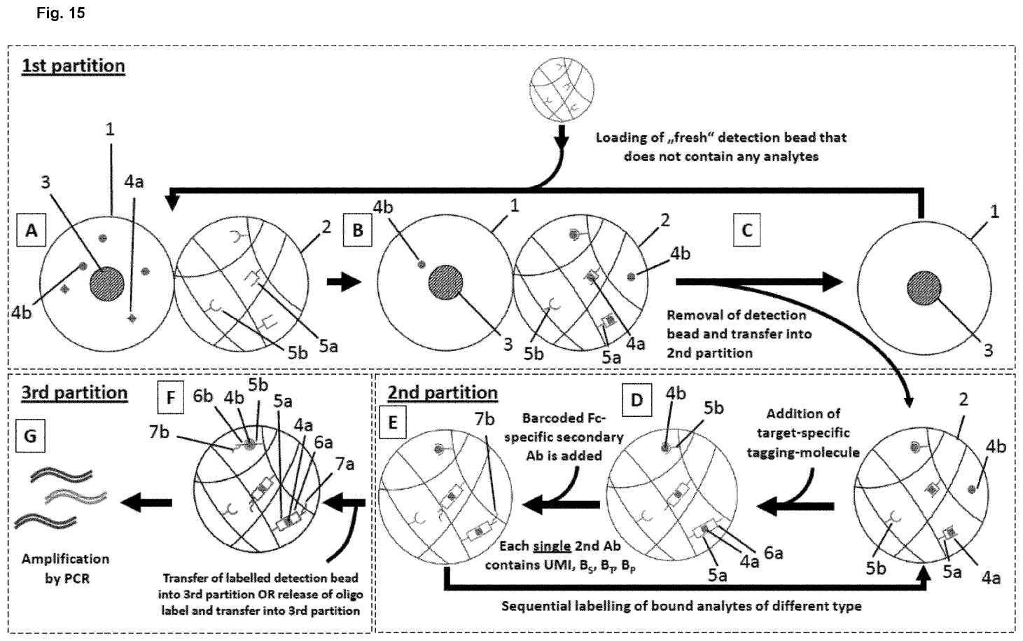

This method is further illustrated in FIG. 15.

As illustrated in FIG. 15, a cell-laden matrix (1), which preferably is a hydrogel bead, and a capture matrix (2), which preferably is a hydrogel bead, are positioned in close proximity within a first partition, preferably an isolated compartment at a position X|Y of a device, which according to a preferred embodiment is a microfabricated cell culture device. The cell-laden matrix comprises in the illustrated embodiment a single cell (3), which secretes two biomolecules of interest (4a and 4b) or more cells which secrete two biomolecules of interest or an empty matrix where one or more cells have been present (e.g. cell(s) may have undergone apoptosis or may escape from the matrix by cell migration) and have secreted two biomolecules of interest. The capture matrix (2) comprises in the illustrated embodiment two different types of capture molecules (5a and 5b), which specifically bind the biomolecules of interest (4a and 4b). The different types of capture molecules are in the illustrated embodiment provided by antibodies with different specificities against the secreted biomolecules of interest. The capture matrix preferably comprises a plurality of capture molecules of the same type to ensure efficient capture of a biomolecule of interest. The capture molecules may be provided in excess of the expected number of secreted biomolecules of interest.

In B, the cell-laden matrix (1) is incubated to allow sufficient secretion of the biomolecules of interest which diffuse from the cell-laden matrix (1) to the capture matrix (2), where a biomolecule of interest is bound by the matching type of capture molecule (see interaction pairs 4a/5a and 4b/5b). Unbound molecules may be washed away.

In C, the capture matrix is transferred from said first partition to a second partition, preferably an isolated compartment at a position X|Y of a device different from the device of the first partition, which according to a preferred embodiment is a microfabricated device. The transfer of the capture matrix from the first partition to the second partition may be performed using a RFCP geometry as described in WO 2019/048713 A1. The capture matrix is trapped within the second partition in a manner that enables individual perfusion and washing of the trapped capture matrix without affecting other capture matrices. Multiple capture matrices might be trapped within multiple partitions of the second partition. The removal of the capture matrix which comprises the complexes comprising the capture molecule and the biomolecule of interest from the first partition leaves the cell-laden matrix in the first partition. As illustrated in C, an “unloaded” capture matrix may be added/loaded into the first partition and a new cycle may be performed at different time-points. The steps may be repeated at several time-points.

In D, after transfer of the capture matrix to the second partition, one type of a tagging-molecule (6a) is added preferably by perfusing the capture matrix within the second partition. The tagging-molecule specifically binds a biomolecule (4a) which is immobilized within the capture matrix by a capture molecule (5a). Afterwards capture matrices are washed to remove any unbound tagging-molecules (6a). In the illustrated embodiment the tagging-molecule (6a) is provided by an antibody which binds the biomolecule of interest at a different epitope than the antibody used for capturing. In other embodiments the tagging-molecule (6a) is provided by a streptavidin-conjugated antibody. Thereby, a complex is formed, comprising the capture molecule, the biomolecule of interest and the tagging-molecule (see complex 4a/5a/6a).

In E, the capture matrix is then perfused with a conjugated detection molecule that specifically binds to the FC region of the tagging-molecule (6a). Importantly, each detection molecule is conjugated to a barcode label which comprises at least (i) the barcode sequence (B_{S}), and (ii) a barcode sequence (B_{T}) for indicating a time information, and/or (iii) a barcode sequence (B_{P}) for indicating position information of the cell-laden matrix, and (iv) optionally a unique molecular identifier (UMI) sequence, and a primer sequence. The barcode label may be provided by an oligonucleotide sequence that may be attached via a linker to the detection molecule. In an embodiment, the linker is provided by a photocleavable spacer. In the illustrated embodiment the detection molecule (7) is provided by an antibody. In other embodiments the detection molecule, in form of a nanobody or biotin is conjugated to said barcode label (7).

In F, the capture matrix is transferred from said second partition to a third partition, preferably an isolated compartment at a position X|Y of a device, which according to a preferred embodiment is a multi-well device such as a 96 well plate. The transfer of the capture matrix from partition 2 to partition 3 may be performed using a RFCP geometry as described in WO 2019/048713 A1. The capture matrix is positioned within partition 3 in a manner that enables individual perfusion and washing of the positioned capture matrix without affecting other capture matrices. Multiple capture matrices might be positioned within multiple partitions of the third partition. In another preferred embodiment all capture matrices are transferred and pooled from said second partition to a reaction tube.

In G, a sequenceable reaction product is generated which comprises at least (i) the barcode sequence (B_{S}), and (ii) a barcode sequence (B_{T}) for indicating a time information, and/or (iii) a barcode sequence (B_{P}) for indicating position information of the cell-laden matrix, and (iv) optionally a unique molecular identifier (UMI) sequence. The generation of the sequenceable reaction product comprises the use of at least one oligonucleotide, which in one embodiment is a universal primer, that is capable of hybridizing to the barcode label of the at least one type of detection molecule.

Preferably, G comprises performing an amplification reaction using a primer or primer combination. In the illustrated embodiment, such amplification reaction is performed after performing step F. The amplification reaction is indicated in FIG. 15 as (G) and comprises performing an amplification reaction with a primer or primer combination. If a single primer is used, a linear amplification can be performed by performing several amplification cycles. The use of a primer combination such as a primer pair allows to perform a PCR reaction. The primer or primer combination as well as the additional components required for performing the amplification reaction (such as a polymerase, dNTPs, buffers) may be added to the compartments (e.g. wells or reaction tube) of the third partition that comprise the transferred capture matrix, or may be provided in advance. As is described herein, the barcode label may be released from the detection molecule in advance of the amplification reaction, e.g. when a photocleavable linker is used.

The amplification products may then be subsequently sequenced.

FIG. 16 shows one embodiment, wherein the barcode label is attached to a capture molecule (e.g. antibody or nanobody) or a biotin moiety, that are capable of binding the tagging molecule that specifically binds to the biomolecule of interest. The biomolecule of interest is loaded onto the capture matrix. The different possible and preferred elements of the barcode label are shown in this embodiment. Preferably, the barcode label comprises all these elements. After binding to the loaded capture matrix, an amplification reaction can be performed followed by a sequencing reaction.

It is further referred to the figures and the description of PCT/EP2020/056975 (priority application number: EP 19 162 691.0), in particular to FIGS. 1, 2B, 2D, 4, 10, 11, 12, 13, 14, 15, 16, 17 19, 20 and 21, described on pages 90 to 110, which are herein incorporated by reference.

Further embodiments and options are disclosed in the appended claims.

In the method of the present invention, different types of matrices are applied, which either are loaded with at least one cell (corresponding to a cell-laden matrix) or with one or more types of capture molecules (corresponding to a capture matrix). The matrices have multiple advantages, including that cells can be cultivated under physiological conditions inside the matrix. On the other hand the capture molecules comprised in the capture matrix can be flexibly attached to the matrix which has an increased surface area for attachment (e.g. in comparison to solid particles, wherein only the surface is available for attachment). Each matrix type can then be advantageously brought into contact or proximity to each other, preferably inside a compartment. Such a compartment may be preferably provided by a microfabricated cell culture device, allowing matrices to be transported into and away from the compartment (and if desired also away from the microfabricated cell culture device into another format (e.g. well plate)). Moreover, the microfabricated cell culture device preferably comprises means to switch the compartment between an open and an isolated state throughout cultivation. For instance, in an isolated compartment, biomolecules of interest that are released by the at least one cell throughout the incubation can diffuse out of the cell-laden matrix after their release. Afterwards, the biomolecules of interest can diffuse to the capture matrix, wherein capture molecules can bind biomolecules of interest, while due to the isolated state of the compartment, the biomolecules are not lost (e.g. due to perfusion or washing). Preferably, each type of capture molecule binds a different biomolecule of interest. Hence, advantageously biomolecules of interest can be captured by the capture matrix, respectively the one or more types of capture molecules. Afterwards, detection molecules are added, wherein each type of detection molecule preferably binds to a different biomolecule of interest, and wherein the molecules of each type of detection molecule comprise a barcode label which comprises a barcode sequence (B_{S}) indicating the specificity of the detection molecule. This has the advantage, that multiple biomolecules of interest can be analyzed in a multiplexed manner, as the barcode label allows for a subsequent differentiation, e.g. by sequencing. This background is also described in PCT/EP2020/056975 (priority application number: EP 19 162 691.0) on p. 3 to 4, herein incorporated by reference.

As is also disclosed PCT/EP2020/056975, the generation of a sequenceable reaction product advantageously allows subsequent pooling of multiple reaction products for sequencing, as these can be differentiated and clearly assigned based on the comprised sequence elements/barcodes. Such differentiation preferably takes place after sequencing the generated reaction product. Hence, a very efficient and multiplexed analysis with a high throughput of cell-released biomolecules is achieved by the provided technology. The generated sequenceable reaction product can optionally comprise a unique molecule identifier (UMI) sequence allowing to analyze the bound molecules in a highly quantitative manner, which is advantageous for an absolute analysis of biomolecules of interest.

Suitable materials for providing the cell-laden matrix are disclosed in PCT/EP2020/056975, herein incorporated by reference. According to the first aspect of the present disclosure, a method is provided for analyzing one or more cell released biomolecules, wherein the cells are provided by a cell-laden matrix. The cell-laden matrix comprises at least one cell that is capable of releasing one or more biomolecules of interest for instance by secretion. According to one embodiment the cell-laden matrix comprises a hydrogel, wherein preferably the matrix material is provided by a hydrogel. According to a preferred embodiment, the at least one cell is encapsulated inside a matrix. A matrix preferably provides a three-dimensional matrix which can advantageously surround the at least one cell. This advantageously provides an environment to the cells that mimics the environment the cells naturally encounter and thus a more physiological environment can be established. For instance, a matrix can be provided that mimics the biochemical, mechanical and structural environment a cell would encounter in nature. In a particular embodiment, a human or human-derived cell may be encapsulated by a hydrogel matrix, which can be advantageously adapted to provide a particular three-dimensional environment to the cells, including one that the cell would encounter in the body in a vital or diseased state. Suitable embodiments for the at least one cell are described herein further below throughout the further embodiments of the method of the first aspect and it is referred thereto. The further below disclosed embodiments can be advantageously applied for the method according to the first aspect.

According to one embodiment, the cell-laden matrix comprises at least one cell. The cell-laden matrix may advantageously comprise a pre-defined cell composition. Such a pre-defined cell composition can be selected from the group comprising a single cell, multiple cells, cell colonies, mini-tissues, mini-organs, tissue samples, and combinations thereof. Other cell compositions (i.e. cell/cells to be provided in form of a cell-laden matrix) are described further below and also apply here. The pre-defined cell composition advantageously enables profiling of secreted molecules from pre-defined cell compositions (arrangements). One result of such an embodiment may be that only secretomes (e.g. the sum of released biomolecules of interest) from cells of interest will be quantified. The embodiment advantageously enables to customize experiments in a cost-effective manner maintaining high data integrity. Furthermore, the cell-laden matrix may have comprised at least one cell that has released one or more biomolecules of interest (but e.g. died due to apoptosis as disclosed herein).

The released biomolecules of interest according to the present disclosure can be a number of different kinds/types of biomolecules. Various biomolecules which may be analyzed in scope of the present invention are disclosed below in the section disclosing the further embodiments of the method of the first aspect and these embodiments also apply here. Particular biomolecules of interest are proteins which may be released by the at least one cell in scope of secretion processes. Exemplary proteins may be cytokines which are secreted by cells and their analysis allows to study the interaction of cells in view of cell-cell communication by cytokines. Such an analysis is important in understanding cellular processes and can contribute to study for instance the interaction between cancer and immune cells to improve immuno-therapies. Various other applications of the present disclosure are feasible and are apparent throughout the present disclosure.

Moreover, the matrices disclosed herein, including the cell-laden matrix and capture matrix, preferably comprise a hydrogel, which may be formed upon the gelation/polymerization/curing of a monomer, pre-polymer, precursor, polymer and/or building block. Particular, monomers, pre-polymers, precursors, polymers and/or building blocks are disclosed below in the further embodiments of the method of the first aspect and can be advantageously applied in order to form matrices of the present disclosure. Suitable embodiments are described herein.

In addition, the matrices disclosed herein, including the cell-laden matrix and capture matrix, may have different shapes. In a preferred embodiment, matrices are formed using droplet microfluidics. For example, a flow focusing geometry can be used for the generation of highly monodisperse droplets having a spherical shape. If the droplet diameter is larger than the width/height of the microfluidic channel in which the hydrogel formation may occur, formed matrices have a plug-like shape. In addition, matrices may be formed by conventional pipetting. Thus, matrix solutions comprising monomers, pre-polymers, precursors, polymer and/or building blocks for gelation/polymerization/curing reactions may be pipetted on a 2D surface resulting in the formation of a droplet having the shape of a spherical segment and/or a hemi-spherical shape. The shape depends on the surface tension between the droplet and the surrounding surfaces and may be adjusted by changing the surface characteristics. In another embodiment, matrix solutions comprising monomers, pre-polymers, precursors, polymer and/or building blocks for gelation/polymerization/curing reactions may be pipetted into a geometry having a pre-defined shape (e.g. a cylindrical geometry). Thus, matrices may assume the shape of the container containing the matrix solution during matrix formation.

According to one embodiment, the volume of matrices disclosed herein, including the cell-laden matrix and capture matrix, may vary depending on the used method for matrix formation. In a preferred embodiment, matrices are formed using droplet microfluidics as described in the present disclosure having a volume within the range of 50 fl to 50 nl, in particular between 200 pl and 400 pl. In one embodiment, matrices may be formed by methods such as conventional pipetting having a volume between 0.5 μl to 500 μl, such as 1 μl to 200 μl or 2 μl to 100 μl. In one embodiment, the volume of a matrix is ≤200 μl, such as ≤100 μl, ≤50 μl, ≤10 μl, ≤1 μl, ≤0.5 μl, ≤300 nl, ≤200 nl, ≤100 nl, ≤50 nl or ≤5 nl, preferably 0.05 μl to 2000 μl;

A microfabricated cell culture device as used herein in particular refers to a device having geometries/structures with size dimensions smaller than 1000 μm while being compatible with the incubation of cells. Said geometries may be fabricated using conventional microfabrication techniques such as lithography, soft lithography, replica molding or techniques such as 3D printing, CNC-milling or injection molding.

According to one embodiment, the matrix of the cell-laden matrix (and/or the capture matrix) is a hydrogel which has one or more of the following characteristics:
a) the hydrogel comprises cross-linked hydrogel precursor molecules of the same type or of different types;
b) the hydrogel is composed of at least two different polymers with different structures as hydrogel precursor molecules, wherein optionally, at least one polymer is a copolymer;
c) the hydrogel is formed using at least one polymer which has a linear structure and at least one polymer which has a multiarm or star-shaped structure;
d) the hydrogel is formed using a t least one polymer of formula (P1)

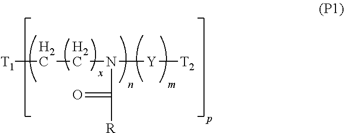

wherein
R is independently selected from a hydrogen atom, a hydrocarbon with 1-18 carbonatoms (preferably CH₃, —C₂H₅), a C₁-C₂₅-hydrocarbon with at least one hydroxy group, a C₁-C₂₅-hydrocarbon with at least one carboxy group, (C₂-C₆)alkylthiol, (C₂-C₆)alkylamine, protected (C₂-C₆)alkylamine (preferably —(CH₂)₂₋₆—NH—CO—R (with R=tert-Butyl, perfluoroalkyl)), (C₂-C₆)alkylazide, polyethylene glycol, polylactic acid, polyglycolic acid, polyoxazoline, or wherein R is a residue R⁴
Y is a moiety containing at least one graft, comprising at least one residue R⁴,
T₁ is a terminating moiety, which may contain a residue R⁴,
T₂ is a terminating moiety, which contains a residue R⁴,
p is an integer from 1 to 10,
n is an integer greater than 1 and preferably, below 500,
m is zero or an integer of at least, preferably greater than 1, and preferably, below 500, the sum n+m is greater than 10,
x is independently 1, 2 or 3, preferably x is independently 1 or 2, most preferably x is 1,
R⁴ independently comprise at least one functional group
   for crosslinking and/or
   for binding biologically active compounds, and
   optionally comprising a (preferably degradable) spacer moiety connecting said functional group with the binding site to the respective moiety of the structure of formula (P1),
   wherein the entirety of all m-fold and n-fold repeating units are distributed in any order within the polymer chain and wherein optionally, the polymer is a random copolymer or a block copolymer.

According to one embodiment, the matrix of the cell-laden matrix and/or the capture matrix is a particle, preferably a spherical particle. According to one embodiment, the matrices disclosed herein are preferably spherical, e.g. spherical hydrogel matrices but other forms may also be applied. Applicable shapes/forms of the matrix (such as a hemi-spherical or plaque-like shape) are described further below and also apply here. According to one embodiment, the matrix has a diameter of ≤1000 μm, such as ≤800 μm, ≤600 μm or ≤400 μm, preferably ≤200 μm, such as 5 μm to 150 μm. Other applicable diameters of the matrix are described below when disclosing the further embodiments of the method of the first aspect. According to a particular embodiment, the cell-laden matrix may be a hydrogel matrix that provides a three-dimensional environment to the at least one cell, wherein preferably the matrix is at least 5 μm and ≤200 μm in diameter.

The individual steps of the method according to the first aspect will be further explained below. It is also referred to the figures which illustrate different exemplary embodiments of the method according to the present disclosure.

Steps a) and b)

In the method, a cell-laden matrix is provided. In step a), a capture matrix is provided, wherein the capture matrix comprises one or more types of capture molecules, wherein each type of capture molecule binds a biomolecule of interest. The cell-laden matrix may be provided such as e.g. prepared using methods disclosed herein.

According to step b), the cell-laden matrix is incubated to allow release of the one or more biomolecules of interest. As is disclosed herein, incubating the cell-laden matrix to allow release (e.g. secretion) of the biomolecules of interest may already occur for a time period before the capture matrix is provided in proximity to the cell-laden matrix in order to allow capture of the released biomolecules of interest by the capture matrix. However, the capture matrix may also be present during the entire incubation process. The one or more biomolecules of interest may diffuse from the cell-laden matrix to the capture matrix, where the one or more biomolecules of interest are specifically bound by the one or more types of capture molecules.

The provided capture matrix preferably comprises a hydrogel, wherein the matrix material is preferably provided by a hydrogel. According to one embodiment, the matrix is three-dimensional. According to a preferred embodiment, the capture matrix comprises a three-dimensional hydrogel. By providing a capture matrix comprising one or more types of capture molecules, a high surface area is provided for attaching the capture molecules, which advantageously allows to provide at least one type of capture molecules at a high number (e.g. in comparison to solid particles, which merely provide the surface of the particle for attaching capture molecules thereto). As disclosed above for the cell-laden matrix, the capture matrix may be formed upon gelation/polymerization/curing of a monomer, pre-polymer, precursor, polymer and/or building block, which are disclosed below in the further embodiments of the method of the first aspect and can be advantageously applied in order to form matrices of the present disclosure. The capture matrix may comprise a crosslinked monomer, pre-polymer, precursor, polymer and/or building block, known from the prior art by the skilled person. Typical polymers of the prior art may be applied, selected from the non-limiting list comprising polyacrylamide (PMA), poly(lactic acid) (PLA), poly(vinyl alcohol) (PVA), polyethylene glycol (PEG), polyoxazoline (POx), and polystyrene (PS). The capture matrix may be formed upon reaction of the same monomer, pre-polymer, precursor, polymer and/or building block or different monomer, pre-polymer, precursor, polymer and/or building block. According to a preferred embodiment, the capture matrix comprises a pore size that allows for the diffusion of at least a portion of the released biomolecules of interest into the matrix. This advantageously allows biomolecules of interest not only to access the surface but also the interior in order to bind to the one or more types of capture molecules.

According to one embodiment, the matrix is a particle, preferably a spherical particle such as a bead. According to one embodiment, the capture matrix is provided in form of a hydrogel matrix, preferably a spherical hydrogel matrix. However the form/shape of the capture matrix is not limited to a spherical shape and other shapes (such as a hemi-spherical or plaque-like shape) are also possible and described below. According to one embodiment, the matrix has a diameter of ≤1000 μm, such as ≤800 μm, ≤600 μm or ≤400 μm, preferably ≤200 μm such as 5 μm to 150 μm. Other applicable diameters of the matrix are described below and also apply here.

According to one embodiment, the capture molecules are attached to the capture matrix in order to be capable of binding a biomolecule of interest and thereby capture the biomolecule of interest in a form which can be freely moved/transported (e.g. the capture matrix can be freely transferred away from the cell-laden matrix). According to an advantageous embodiment, the capture matrix comprises at least one type of capture molecules, which can be in an exemplary embodiment at least one type of antibody with a defined specificity for a biomolecule of interest. Biomolecules of interest that are released by the at least one cell can be immobilized within the capture matrix of the present disclosure, which enables single- and multiplexing within one experiment in a highly customizable manner, as the capture molecules specifically bind to biomolecules of interest derived/released from the cell-laden matrix. Applicable capture molecules are also disclosed below in conjunction with the further embodiments of the method according to the first aspect. Exemplary capture molecules include but are not limited to antibodies, antibody fragments, aptamers, etc. One preferred capture molecule may be a capture molecule derived from an antibody. According to a preferred embodiment, one type of capture molecules comprises multiple capture molecules of the same type. Therefore, advantageously multiple biomolecules of interest of the same type can be captured, e.g. in order to perform quantitative analysis.

According to one embodiment, the capture matrix comprising the one or more types of capture molecules is positioned after a pre-defined cultivation/stimulation period. The capture matrix may also be provided together with the cell-laden matrix or shortly afterwards, in some embodiments even before the cell-laden matrix. In an embodiment, wherein the different matrices are provided shortly after each other, preferably first the cell-laden matrix is provided and then the capture matrix, so that the released biomolecules of interest can be directly bound by the one or more types of capture molecules of the capture matrix. Therefore, biomolecules of interest are first released by the at least one cell (e.g. over an incubation period) and diffuse out of the cell-laden matrix to the capture matrix. Accordingly, the matrix material of the cell-laden matrix and the capture matrix are preferably selected such that the biomolecules of interest can diffuse through the matrices and preferably do not non-specifically interact with/bind to the matrix material, so that the biomolecules of interest can be efficiently captured by the one or more types of capture molecules. Hence, it is advantageous to limit the binding of biomolecules of interest to specific binding to the capture molecules (while a certain degree of unspecific binding may occur but is not preferred). After exiting the cell-laden matrix, the biomolecules of interest diffuse to and preferably into the capture matrix, wherein the one or more biomolecules of interest are specifically bound by the one or more types of capture molecules. The capture matrix is preferably tailored such that the one or more biomolecules of interest can diffuse into the matrix and do not (or non-significantly) non-specifically interact with/bind to the matrix material. Preferably, the one or more biomolecules of interest specifically interact with the capture molecules.

Throughout incubation, artificial “capture effects” can be avoided by the present method, as released biomolecules of interest need to first diffuse out of the cell-laden matrix and then diffuse into the capture matrix to be bound by the at least one type of capture molecules. Typically, the biomolecules also need to diffuse through a part of the surrounding fluid before diffusing onto/into the capture matrix. Therefore, the biomolecules of interest are slowly removed from the cell-laden matrix and hence the cells). In contrast, prior art methods for analysis of biomolecules of interest predominantly do not comprise a matrix that surrounds the cell(s), so that biomolecules of interest are directly and quickly captured. This can impede a physiological cell response, e.g. intra-cellularly or inter-cellularly, e.g. between two cells to be analysed. Hence, the at least two matrices used according to the present invention allow to provide physiological environments (including a physiological cell signalling) in order to establish of dynamic cell-cell-interactions and autocrine signalling. The subsequent quantification of secreted biomolecules advantageously can prevent capture effects.

The Cell Culture Device

According to a preferred embodiment, the provision of a cell-laden matrix and a capture matrix is performed utilizing a cell culture device. Preferably, the cell culture device is a microfabricated cell culture device. According to one embodiment, the cell culture device comprises at least one compartment, preferably an array of compartments, wherein an array corresponds to a plurality of compartments that are ordered. Preferably, the order of the plurality of compartments comprises positions of x- and y-coordinates (e.g. n×m array). An exemplary plurality of compartments (also referred to as an array) is depicted in FIG. 1 of PCT/EP2020/056975 and it is referred thereto. Other orders including no order (e.g. random order) of compartments are also within scope of the present disclosure.

The at least one compartment is typically connected to channels, through which transport can be performed. According to a preferred embodiment, the device comprises a fluid reservoir and fluid channels for providing fluid to the at least one compartment. For instance, fluid can be transported through the channels into the compartments and then further out of the compartments to either a subsequent compartment (e.g. following compartment of a plurality of compartments) or a waste or a reservoir. The fluid may also be transported to other positions of the cell culture device (e.g. a storage position, etc.). In addition to the fluid (which can be an aqueous fluid, such as cell culture media, or a non-aqueous fluid, such as fluorinated oil), other components can be transported through the channels (e.g. inside the fluid). According to a preferred embodiment, matrices, including cell-laden matrices and capture matrices can be transported through the channels. Moreover, cell suspensions and solutions which are capable of crosslinking or being crosslinked can be transported through the channels. Hence, the at least one (microfabricated) compartment can be perfused with different solutions in a controlled manner, allowing for instance, washing of the matrices and perfusion with cell media. For instance, washing may be beneficial in scope of the method of the first aspect in order to remove unbound molecules which would give false positive results if not removed. According to one embodiment, a plurality of compartments is provided in an array, wherein compartments can advantageously share common inlets (e.g. feeling line), allowing matrices (e.g. the capture matrices) being positioned using one feeding line. This advantageously increases the speed of the disclosed method.

According to one embodiment, at least one cell is first encapsulated by utilizing the cell culture device in one droplet, which forms the matrix after droplet generation. Also the capture matrix may be formed by generating a droplet utilizing the cell culture device, followed by matrix generation of the droplet. A formed cell-laden matrix and/or capture matrix may then be transported through the channels to a compartment, wherein the matrices can be positioned in proximity to each other (also referred to as accommodated). According to one embodiment, the device comprises at least one compartment for accommodating at least one, preferably at least two matrices, including at least one capture matrix and/or at least one cell-laden matrix. According to one embodiment, the device comprises a compartment for accommodating at least one matrix, preferably two matrices, wherein a microfabricated geometry for matrix immobilization is present suitable for positioning the at least one matrix. According to one embodiment, a plurality of compartments for accommodating at least one matrix, preferably by an array of compartments is comprised in the cell culture device.

According to a preferred embodiment, a plurality of cell-laden matrices and capture matrices are provided in a cell culture device comprising a plurality of compartments, wherein at least one cell-laden matrix and at least one capture matrix are provided within a compartment of the cell culture device. According to a preferred embodiment, the cell-laden matrix and the capture matrix are provided in proximity within a compartment of a device. Alternatively, the cell-laden matrix and the capture matrix are provided in separate compartments, wherein the separate compartments are in fluid communication with each other or can be brought in fluid communication with each other (e.g. by the operation of a valve) so that the released biomolecules of interest can contact the capture matrix for capturing. According to a preferred embodiment, the cell-laden matrix and capture matrix are located in proximity, preferably in close proximity, at a defined position (e.g. in one compartment, in particular in a microfabricated compartment at position (n|m) of an n×m array of microfabricated compartments). This is particularly advantageous, as the proximity between said matrices allows for diffusion of biomolecules of interest between the said matrices (e.g. released biomolecules of interest derived from single or multiple cells located within the cell-laden matrix can diffuse through the matrix to the neighboring capture matrix). Hence, it is possible to achieve high capture efficiency of released biomolecules of interest due to short diffusion distances between two matrices. Moreover, the theoretical reduction of reaction volume can increase the sensitivity by increasing concentrations (e.g. by increasing the local concentration and thus capturing efficiency). According to a particular embodiment, the reaction volume may be further reduced by replacing an aqueous phase that may surround the matrices (in particular the cell-laden matrix) with a water-immiscible phase (e.g. oil phase) to generate a matrix comprising a shell of said water-immiscible phase (e.g. alternating biphasic compartment generation). Thereby advantageously, the reaction volume can be reduced to increase locally the concentration of the biomolecules of interest as these are hindered in diffusing out of the matrix by said shell. Furthermore, a capture matrix may be provided in close proximity (e.g. in direct contact to the cell-laden matrix) to enable diffusion from the cell-laden matrix. Hence, a detection mechanism with higher sensitivity may be achieved. According to another embodiment, the cell-laden matrix and capture matrix can be separated from each other by distance and/or time (e.g. by providing the capture matrix and cell-laden matrix in different compartments). Accordingly, at least one cell-laden matrix and at least one capture matrix are positioned preferably by a microfabricated geometry for matrix immobilization inside a compartment, wherein the compartment accommodating the at least one cell-laden matrix is different from the compartment accommodating the at least one capture matrix and wherein both compartments can be switched to be either in fluid contact with each other or to be in no fluid contact with each other. In order to still allow capture of biomolecules of interest, the cell-laden matrix and capture matrix may be provided in neighboring compartments that can be selectively brought into fluid contact with each other (e.g. by a valve, preferably a microfabricated valve). Thereby, advantageously, cell cultivation under physiological conditions is separated from further reactions (e.g. from step c) and/or d)). Moreover, capture effects may be avoided ensuring physiological environments (for improved signaling).

In addition, the matrices can be selectively removed from the compartment (e.g. the capture matrix can be independently from the cell-laden matrix transported in and out of the compartment). According to one embodiment, the method comprises obtaining capture matrices from a plurality of compartments and transfer of the capture matrices to a device comprising a plurality of compartments. For instance, the capture matrix may be removed from a compartment, while the cell-laden matrix stays inside the compartment, whereupon the capture matrix can be transported to a storage position (also referred to as a compartment of a device which is not the compartment comprising the cell-laden matrix). Another capture matrix can then be added if desired. Moreover, a capture matrix within a compartment located within an array can be removed without removing capture matrices located within other compartments of an array of compartments. Thereby, capture matrices can be obtained in a controlled manner and the position information is advantageously preserved (e.g. by selectively obtaining capture matrices of a particular compartment and transferring the capture matrix into a separate well of a well plate). In case multiple capture matrices are obtained, each capture matrix is transferred to a storage position, wherein the storage positions of matrices from different compartments are preferably different. The storage position may be any position capable of storing a matrix, including a position on the cell culture device (it was initially provided to) or a position outside of the cell culture device (e.g. by transporting the matrix outside the cell culture device into another format, such as a well of a well plate, e.g. a collection well). Transferring the capture matrix to a storage position at which the capture matrix can be perfused independently from the cell-laden matrix has the advantage, that the capture matrix can be washed/processed without affecting the cell-laden matrix/matrices. Thus, the cell behaviour, respectively the cell-laden matrix is not affected by processing of the capture matrix.

According to one embodiment, after obtaining a capture matrix from a compartment comprising the cell-laden matrix, another capture matrix comprising one or more types of capture molecule (but preferably no biomolecules of interest bound thereto) may be provided and transferred to said compartment. Positioning and transfer of fluids and other components (e.g. the matrices) may be performed utilizing a cell culture device comprising compartments comprising a trapping geometry. According to a preferred embodiment, the device comprises a microfabricated geometry for matrix immobilization inside a compartment for releasably positioning at least one matrix.

According to one embodiment, the device comprises a microfabricated geometry for matrix immobilization inside a compartment, wherein the geometry for matrix immobilization has one or more of the following characteristics:
it is capable of positioning the cell-laden matrix and the capture matrix in proximity;
it is capable of positioning at least two cell-laden matrices and the capture matrix in proximity;
it is capable of positioning the cell-laden matrix and at least two capture matrices in proximity; or
it is capable of positioning at least two cell-laden matrices and at least two capture matrices in proximity.

The trapping geometry (which may also be referred to as a positioner or positioning means), enables immobilization of single or multiple matrices (including two, three, four or more matrices, preferably two or three matrices) in a pre-defined/controlled manner. Such a configuration advantageously enables positioning of a capture matrix and a cell-laden hydrogel matrix in a very controlled manner within the same compartment thereby allowing the capture of released biomolecules of interest. According to one embodiment, multiple cell-laden matrices and capture matrices are provided and positioned (preferably at least one of each matrix kind) by a trapping geometry inside a compartment, wherein multiple such compartments are provided (i.e. in an array). Such an embodiment advantageously allows to quantify simultaneously/in parallel released biomolecules of interest from pre-defined cell compositions provided by the cell-laden matrices in a highly multiplexed manner.

The trapping geometry may comprise a valve arrangement adapted to provide a fluid passing through a microfabricated geometry for matrix immobilization. In one embodiment, the device comprises a trapping geometry comprising a valve arrangement adapted to provide a fluid passing through a microfabricated geometry for matrix immobilization wherein the valve arrangement is adapted to selectively change the direction of fluid passing the microfabricated geometry for matrix immobilization, in particular wherein a fluid a first direction urging the at least one matrix into the microfabricated geometry for matrix immobilization and a fluid in the second direction urging the at least one matrix out of the microfabricated geometry for matrix immobilization, and in particular fluid in the second direction delivering the at least one matrix in direction of an exit section. Such a configuration can advantageously transfer one or more matrices inside the compartment. Such a configuration can further advantageously be utilized for obtaining one or more matrices from the compartment. The mechanism of such a configuration may rely or may also be referred to as a reverse flow cherry picking (RFCP) mechanism, as is described e.g. in WO 2019/048713 A1. Such a valve arrangement allows to transfer fluid which may comprise individual matrices from and into a compartment. Therefore, capture matrices can be transferred after a pre-defined period into another format, while the position information is maintained allowing to correlate the release profile of biomolecules of interest with the corresponding cell(s).

According to a preferred embodiment, the valve arrangements are based on microfabricated valves. These are disclosed below and also apply here. A microfabricated valve may be capable of switching the compartment to an open or closed state. According to one embodiment, a microfabricated valve comprises a first channel, a second channel, a connection channel connecting the first channel and the second channel, a valve portion arranged within the connection channel, wherein the valve portion is adapted to selectively open and close the connection channel. According to one embodiment, a microfabricated valve comprises at least three layers, wherein a first channel is located within a first layer; a second channel is located within a third layer; a valve portion is located within a second layer; the second layer is arranged between the first and the third layer. A device may comprise a microfabricated valve, wherein a first channel comprises a microfabricated geometry for matrix immobilization suitable for positioning at least one matrix being contained in a fluid which flows through the first channel, wherein the microfabricated geometry for matrix immobilization is arranged within the first channel in such a way that a fluid flow can be reduced by the microfabricated geometry for matrix immobilization, in particular, the microfabricated geometry for matrix immobilization narrows the cross section of the channel; and/or wherein a second channel comprises a microfabricated geometry for matrix immobilization suitable for positioning particles being contained in a fluid which flows through the second channel, wherein the microfabricated geometry for matrix immobilization is arranged within the second channel in such a way that a fluid flow can be reduced by the microfabricated geometry for matrix immobilization, in particular, the microfabricated geometry for matrix immobilization narrows the cross section of the channel.

In order to continuously provide nutrition to the cell-laden matrix (and thus to the at least one cell), the compartments, wherein the cell-laden matrix is accommodated, may be switched to an open state to provide fluid to the compartment. Alternatively, the compartment may be switched to a closed state (also referred to as an isolated state) to provide an isolated compartment, which has the advantage to not flush away secreted biomolecules of interest. Moreover, an isolated compartment can hold both the cell-laden matrix and the capture matrix inside the compartment (e.g. in a defined volume, i.e. closed reaction volume), wherein released biomolecules of interest are not lost, due to removal or exchange of fluid. Therefore, preferably isolated compartments are provided throughout step b), preventing loss of one or more biomolecules of interest. Yet, the method may not be limited to isolated compartments, as also compartments in the open state may be applicable, e.g. in case the flow rate is kept low enough or no flow is provided, allowing biomolecules of interest to remain in proximity to the cell-laden matrix and/or the capture matrix. According to a preferred embodiment, released biomolecules of interest diffuse within the isolated compartment and can be advantageously captured within the compartment for further analysis/processing. According to one embodiment, the device comprises at least one compartment that is capable of being switched between an isolated and an open state, wherein the isolated state corresponds to a state at which fluid that is present in the compartment is in no contact with fluid not present in the compartment and wherein the open state corresponds to a state at which fluid that is present in the compartment is in contact with fluid not present in the compartment.

The cell-laden matrix is preferably incubated to allow release of the one or more biomolecules of interest as is also described in PCT/EP2020/05697, herein incorporated by reference. Incubation may occur over a defined time period. Preferably, the incubation is performed by utilizing a cell culture device, which is preferably a microfabricated cell culture device. The cell-laden matrix, in particular, the at least one cell can be incubated inside a compartment of the cell culture device. Therefore, suitable conditions for incubating/cultivating are preferably applied, including but not limited to supply of one or more of a suitable temperature (e.g. 37° C. for human cells), CO₂-level (e.g. around 5% for human cells) and humidity. As disclosed herein, such incubation may also occur before the capture matrix is added for binding the released biomolecule(s) of interest.

According to one embodiment, the provided cell-laden matrix and capture matrix are provided with a fluid, preferably a fluid that is immiscible with water, wherein said matrices, provided with said fluid, are preferably generated by utilizing a cell culture device, which preferably is a microfabricated cell culture device, and preferably by
(i) releasably positioning the cell-laden matrix and the capture matrix by a preferably microfabricated geometry for matrix immobilization inside a compartment, wherein the compartment comprises a first fluid, preferably an aqueous fluid;
(ii) removing the first fluid from the compartment and replacing the first fluid by a second fluid that provides said fluid, wherein said fluid is preferably immiscible with water; and
(iii) optionally, removing the second fluid from the compartment and replacing it by the first fluid or a third fluid, that is preferably immiscible with the second fluid.

According to one embodiment, the cell-laden matrix and the capture matrix may be confined in a volume that is smaller than the volume of a compartment, in which the matrices may be advantageously positioned (e.g. by a microfabricated geometry for matrix immobilization). This embodiment is described in PCT/EP2020/056975 (see e.g. claims 23 and 24 and associated description), herein incorporated by reference.

According to one embodiment, the cell-laden matrix is incubated to allow release (e.g. secretion) of one or more biomolecules of interest before providing the capture matrix in step (a). After providing the capture matrix, one or more biomolecules of interest are specifically bound by the one or more types of capture molecules of the capture matrix. The cell-laden matrix is preferably provided in a defined volume of a fluid, preferably a fluid that is immiscible with water, and wherein the capture matrix is provided in a defined volume of the same type of fluid, and wherein after contacting the cell-laden matrix and the capture matrix said fluids of the same type merge to provide a defined volume of fluid that is shared by the cell-laden matrix and the capture matrix. As disclosed herein, the capture matrix may be positioned in contact with the cell-laden matrix before, during or after such incubation.

According to one embodiment, the cell-laden matrix is incubated before providing the capture matrix in step (a). Such an incubation step can be performed by utilizing a cell culture device, preferably a microfabricated cell culture device. The incubation can take place as described herein. According to a preferred embodiment, the cell-laden matrix is provided in a defined volume of a fluid (also referred to as second fluid), wherein said fluid is immiscible with a first fluid, wherein the first fluid is the fluid present in the cell-laden matrix, which is preferably an aqueous fluid (e.g. cell culture medium). Preferably, the second fluid is immiscible with water. Thereby, the available volume accessible for the released biomolecules of interest for diffusion is reduced to the volume of the matrices (respectively the first fluid comprised in the matrices) and optionally, the volume of the first fluid that still surrounds the matrix (e.g. in form of a shell, e.g. water-shell or aqueous shell). According to one embodiment, the capture matrix is provided in step a) after incubating the provided cell-laden matrix in a defined volume of a fluid (e.g. second fluid). The incubation may be performed by utilizing a cell culture device comprising a microfabricated geometry for matrix immobilization, wherein preferably the cell-laden matrix is immobilized and is provided inside the second fluid. After incubating the cell-laden matrix, a capture matrix may be preferably provided in a defined volume of the same type of fluid. In this case, the same type of fluid corresponds to the type of fluid the cell-laden matrix was provided in/with. The defined volumes of the fluid (e.g. second fluid) that the matrices are provided in/with may be any volume of fluid as long as the volume of fluid is capable of at least partially surrounding the matrices. In a preferred embodiment, the volume of fluid is capable of fully surrounding the matrices. After providing the capture matrix provided in a defined volume of the same type of fluid, said capture matrix, respectively capture matrix provided in a defined volume of the same type of fluid is contacted with the cell-laden matrix, provided in a defined volume of the fluid (e.g. second fluid). When the matrices contact each other, respectively the provided fluids of the same type (e.g. second fluids), the fluids merge (which may also be referred to as coalesce) to provide a defined volume of fluid that is shared by the cell-laden matrix and the capture matrix. The shared volume may be any volume as long as it is at least partially, preferably fully, capable of surrounding said matrices. It may correspond to the sum of the defined volumes provided or may be less than the sum or more than the sum. The capture matrix may be provided such that the cell-laden matrix and the capture matrix are in close proximity. Preferably, the cell-laden matrix and the capture matrix are in direct contact with each other, advantageously allowing for a direct diffusion of biomolecules of interest from the cell-laden matrix to the capture matrix. A direct contact between the cell-laden matrix and the capture matrix may be established by the microfabricated geometry for matrix immobilization inside a compartment. For instance, a cell-laden matrix may be positioned directly next to a capture matrix by the microfabricated geometry for matrix immobilization inside a compartment. The delayed provision of the capture matrix has the advantage that released biomolecules of interest can first accumulate within the cell-laden matrix provided in a defined volume of the second fluid (e.g. biomolecules accumulate in the aqueous fluid present in the cell-laden matrix and optionally a surrounding aqueous shell, which may also be referred to as the first fluid), as the biomolecules of interest may be less soluble in the second fluid, which is immiscible with the first fluid (e.g. immiscible with water). In a particular embodiment, more than one cell-laden matrix is provided in a defined volume of a fluid (e.g. second fluid). These may be preferably in close proximity, more preferably in direct contact with each other to allow for a direct diffusion of biomolecules of interest between the cell-laden matrices. For instance two cell-laden matrices or three cell-laden matrices may be provided, wherein the at least one cell of each cell-laden matrix can be of the same type or different. In a particular example, the cells may be different in order to study their interaction. The provision of the cell-laden matrices provided in a defined volume of a fluid (e.g. second fluid) allows for a paracrine and autocrine signaling of cells and avoiding capture effects (which can occur when biomolecules of interest are directly captured; see disclosure above for further details about the capture effect). Only when the capture matrix is provided and positioned in close proximity, preferably in direct contact, with one or more cell-laden matrices, the released biomolecules of interest bind to the one or more type of capture molecules.

In one embodiment the cell culture device is a conventional cell culture flask or cell culture plate such as a 12-well plate, a 24-well plate, 96-well plate, a 384-well plate.

According to one embodiment, the cell-laden matrix is provided in a cell culture device selected from a cell culture flask or cell culture plate, such as a 12-well plate, a 24-well plate, 96-well plate, a 384-well plate. In one embodiment, the cell-laden matrix is provided in a compartment of the cell culture plate, e.g. a well. Per compartment, one or more cell-laden matrices (e.g. 1, 2, 3 or more, optionally 1) may be provided. The cell-laden matrix can be positioned in the compartment of the cell culture plate such that liquid which may surround the cell-laden matrix can be exchanged without affecting the cell-laden matrix, e.g. without contacting or disrupting the cell-laden matrix. For instance, the cell-laden matrix can be provided inside the compartment of the cell culture plate leaving an outer rim allowing liquid to be accessed, e.g. by a pipette, without affecting the cell-laden matrix. The cell-laden matrix is optionally incubated before being in fluidic contact with the capture matrix. For instance, the capture matrix may placed in a separate compartment or containment (e.g. tube). After incubating the cell-laden matrix to release the biomolecule(s) of interest, the capture matrix may be added for binding the released biomolecule(s) of interest. First incubating the cell-laden matrix prior to contacting the released biomolecule(s) of interest with the capture matrix allows that the released biomolecule(s) of interest may first accumulate in the compartment of the cell culture plate before binding to the one or more types of capture molecules of the capture matrix. However, different contacting orders of capture matrix and cell-laden matrix are possible and within the scope of the present disclosure, as disclosed herein.

According to one embodiment, the cell-laden matrix is analyzed by optical analysis throughout the incubation, wherein methods and devices for optical analysis are well-known in the art. An exemplary optical analysis may be microscopic analysis. In a preferred embodiment, optical analysis can be advantageously performed in combination with the cell culture device (e.g. the cell-laden matrix can be optically analyzed by microscopy when present in the cell culture device, preferably present in a compartment of a cell culture device).

Further details of the here described subject-matter, including the cell culture device, channels, microfabricated valve, and valve arrangement are disclosed further below in the further embodiments of the method of the first aspect and it is here referred to the respective disclosure which also applies here.

It is also within the scope of the present disclosure, to transfer the capture matrix to a storage position, whereupon the one or more types of detection molecules are added. In such an embodiment, the direct contact between the one or more type of detection molecules and the cell-laden matrix can be avoided.

The addition of the one or more types of detection molecules is in one embodiment performed by utilizing the cultivation device, which is preferably a microfabricated cultivation device. The one or more types of detection molecules may be introduced into the cultivation device and in particular into the compartments accommodating the one or more cell-laden matrices and capture matrices. This has the advantage to directly provide the one or more types of detection molecules to the capture matrices without requiring transferring the capture matrix before addition of the detection molecules (which is however, an option). Moreover, by directly introducing the one or more types of detection molecules, only small volumes of liquid may be required, as the compartments and channels of the cell cultivation device are relatively small in comparison to standard cell culture dishes. Hence, material is saved. Introducing detection molecules in such a manner has the further advantage of enabling multiplexing, wherein two or more types of detection molecules can be introduced in a multiplexed manner—without the necessity to perform further liquid handling processes.

According to a one embodiment, the compartment comprising one or more matrices (preferably, the at least one cell-laden matrix and the at least one capture matrix) is washed by perfusion with a washing solution to remove unbound compounds. Compartments may also be washed which do not comprise a matrix (e.g. in form of a pre-perfusion). According to a particular embodiment, the capture matrix in proximity to the cell-laden matrix is washed after a pre-defined incubation time with a solution comprising one or more types of detection molecules. As a result the binding equilibrium of biomolecules of interest is simultaneously achieved to washing and no additional incubation time is required (in comparison to prior art methods, such as an external assay), which can be more time-efficient.

The number of types of detection molecules may be the same as the number of types of capture molecules or may be different. Preferably, the detection molecules are used in excess to allow efficient and quantitative binding of the captured biomolecules of interest.

The detection molecule comprises or consists of a barcode label. The barcode label preferably comprises or consists of an oligonucleotide. In one embodiment, the barcode label comprises DNA, RNA, PNA, LNA or combinations thereof. Preferably, the barcode label comprises a DNA sequence. The attachment between the barcode label and any capture or binding molecule as described herein and in the claims may be covalent and preferably is cleavable (e.g. photocleavable). Such an embodiment has the advantage, that the barcode label can be easily separated and thus quickly accessible for further analysis or processing.

As is described herein and in the claims, the method according to the present invention allows to generate a sequenceable reaction product that comprises a barcode sequence B_{P} for indicating position information of a cell-laden matrix analysed, wherein a sequenceable reaction product is generated for a cell-laden matrix comprised in a compartment that differs in its barcode sequence B_{P} from the barcode sequence B_{P} of the sequenceable reaction product(s) generated for a cell-laden matrix comprised in another compartment. As discussed herein, the barcode sequence B_{P} may e.g. be comprised in the barcode label of the detection molecule or may be introduced during the barcode label extension or during an amplification reaction.

As is described herein and in the claims, the method according to the present invention allows to generate a sequenceable reaction product that comprises a barcode sequence (B_{T}) for indicating a time information and wherein n cycles of the method (preferably steps a) to d)) are performed at different time points tx, wherein n is at least 2 and x indicates the different time points, and wherein for each cycle a sequenceable reaction product is generated that differs in its barcode sequence B_{T} from the barcode sequence B_{T} of all other performed cycles. Such an embodiment advantageously comprises obtaining more than one capture matrix from the same compartment repeatedly. In particular, a capture matrix may be incubated together with the cell-laden matrix for a particular time interval. At the time point t₁, the capture matrix may be obtained from the compartment and transferred to a storage position, whereas the cell-laden matrix remains inside the compartment. Furthermore another capture matrix (also referred to as new capture matrix or “fresh” capture matrix), can be positioned next to the remaining cell-laden matrix inside the compartment. Released biomolecules may be repeatedly bound by the capture molecules of the capture matrix, which advantageously allows to acquire time-lapse secretion profiled of biomolecules of interest. According to one embodiment, the steps a) to d) are performed more than one time. Therefore, the capture matrix is preferably transferred after a defined time interval, which is further disclosed below and also applies here, into a storage position or further processed and a “fresh” capture matrix is transferred into the compartment comprising the cell-laden matrix. Therefore, advantageously the disclosed reverse flow cherry picking may be applied. The steps may be performed more than one time, preferably two times, three times, four times, more preferably ≥five times.

As discussed herein, the barcode sequence B_{T} may e.g. be comprised in the barcode label of the detection molecule or may be introduced during the barcode label extension or during an amplification reaction.

The sequenceable reaction product comprising different barcode sequences (B_{T}) for indicating a time information as disclosed herein can also be obtained using a cell culture device which is a cell culture plate, such as a 12-well plate, a 24-well plate, 96-well plate, a 384-well plate. Such an embodiment advantageously comprises obtaining more than one capture matrix from the same compartment of the cell culture plate, e.g. well. In line, at the time point t₁, the capture matrix may be obtained from the compartment of the cell culture plate, e.g., well, and optionally transferred, whereas the cell-laden matrix remains inside the compartment. Furthermore, a fresh capture matrix can be added to the remaining cell-laden matrix inside the compartment of the cell culture plate. Released biomolecules may be repeatedly bound by the capture molecules of the capture matrix, which advantageously allows to acquire time-lapse secretion profiled of biomolecules of interest. Incubation of the cell-laden matrix in the compartment of the cell culture plate can take place for a time interval selected from ≥10 min, ≥20 min, ≥30 min, 1 h, ≥2 h, ≥3 h, ≥4 h, 5 h or more, up to days 1 d, 2 d or several days. According to one embodiment, the time interval is selected from the range of 30-120 min. The repeated incubation and binding can be performed multiple times, e.g. ≥two times, ≥three times, ≥four times, more preferably ≥five times.

According to a preferred embodiment, the method comprises sequencing the generated sequenceable reaction product(s).

The method may comprise pooling generated sequenceable reaction products from different cycles and/or generated from different compartments and sequencing the obtained pool. According to one embodiment, a plurality of sequenceable reaction products (e.g. an oligonucleotide library) is generated comprising a library of different barcode labels (e.g. oligonucleotides) that contain a barcode sequence B_{S}, a barcode sequence B_{T} and/or a barcode sequence B_{P}, and optionally a quantity information (UMI), as well as optionally, adapter sequences for sequencing. In such a case, the sequenceable reaction product can advantageously encode the required information to correlate the sequencing results to the respective at least one cell (barcode sequence B_{P}), the time point (barcode sequence B_{T}), the biomolecules of interest (barcode sequence B_{S}) and the quantity of biomolecules of interest (UMI sequence). In such a case the generated sequenceable reaction products can be pooled (e.g. a defined fluid volume is taken from each generated sequenceable reaction product and put together, e.g. in one reaction tube) and sequenced/sent for sequencing. Hence, a few or even a single NGS samples may be obtained for sequencing, which is cost effective, as it saved the amount of required sequencing components (e.g. primer, reagents, enzymes, etc.). Further embodiments are described herein.

Prior to sequencing, it is advantageous to perform an amplification reaction to acquire multiple copies of the optionally extended barcode label. Accurate quantification is still possible if UMI sequences are used.

The generated sequenceable reaction product(s), which may be preferably pooled, can be sequenced using any sequencing method. In case further steps to modify the sequenceable reaction product (e.g. by addition of particular adapters) are required in order to be sequenceable by a particular sequencing technologies, these may either be performed throughout step d) of the presently disclosed method or may be subsequently performed in frame of the sequencing protocol of the applied sequencing technology. The disclosed method may not be limited by the particularly applied sequencing technology.

Preferably, sequencing is performed by next generation sequencing (NGS). Prior art next-generation sequencing approaches are reviewed e.g. in Goodwin et al., Nature Reviews, June 2016, Vol. 17: pp. 333-351 “Coming of age: ten years of next-generation sequencing technologies”, Yohe et al., Arch Pathol Lab Med, November 2017, Vol. 141: pp. 1544-1557 “Review of Clinical Next-Generation Sequencing” and Masoudi-Nejad, Chapter 2 “Emergence of Next-Generation Sequencing in “Next Generation Sequencing and Sequence Assembly” SpringerBriefs in Systems Biology, 2013, all herein incorporated by reference. Widely used and here applicable sequencing approaches are referred to such as sequencing by synthesis (SBS) and sequencing by ligation (SBL). SBS includes following non-limiting sequencing technologies: cyclic reversible termination (e.g. Illumina, QIAGEN) and single-nucleotide addition (IonTorrent). SBL includes following non-limiting sequencing technologies: SOLiD and complete Genomics. Non limiting, further applicable sequencing technologies include DNA microarrays, Nanostring, qPCR, optical mapping, single-molecule real-time (SMRT) sequencing (e.g. Pacific Biosciences), Oxford Nanopore Technologies.

The method may furthermore comprise a step of evaluating the obtained sequencing data. The analysis of the sequencing data can advantageously be performed to correlate the obtained sequencing data with information about the one or more biomolecules of interest.

In particular, the analysis of the obtained sequencing data based on the core sequence elements described herein allows to correlate the obtained sequencing data with the type of released biomolecule of interest (e.g. by the sequencing data obtained from the B_{S} sequence element). Moreover, a further correlation can be drawn on the number of biomolecules of interest released (e.g. by the sequencing data obtained from the UMI sequence element), the time point of cultivation at which the capture matrix was obtained (e.g. by the sequencing data obtained from the B_{T} sequence element), and/or the position of the compartment, respectively the position of the cell-laden matrix and/or the capture matrix (e.g. by the sequencing data obtained from the B_{P} sequence element). The number of further correlations that can be drawn depend on the sequence elements that were incorporated into the generated sequenceable reaction product. The results may be plotted in form of a diagram, e.g. quantity of the one or more biomolecules of interest over time, for the different positions/cell-laden matrices.

As disclosed herein, the generated sequenceable reaction products may be pooled in order to generate a pooled sequenceable reaction product for sequencing. The differentiation of the sequencing reaction products comprised in the pool into the individual sequencing reaction products may be performed on the basis of the sequence elements which are incorporated into the sequenceable reaction products of the individual sequencing reaction products. Thus, advantageously, based on the sequencing data, an analysis algorithm can be employed to extract the sequencing information and to determine on such a basis the concentration/number of the biomolecules of interest. The method of analysis (which may also be referred to as an analysis algorithm) can vary depending on the provided sequence elements incorporated into the generated sequenceable reaction product and depending on the generated pool. Ideally, the method of analysis should be capable of differentiating the individually captured biomolecules of interest by the sequencing information of the (pooled) sequenceable reaction products.

According to one embodiment, the analysis algorithm first identifies and categorizes, if present, the barcode sequences B_{P}, then the barcode sequences B_{T}, then the barcode sequences B_{S}, then the UMI sequence. Thus advantageously, the position of the compartment, the time point of cultivation, the biomolecule of interest and the number of biomolecules of interest ca be determined, if said data was incorporated into the sequenceable product when performing the method.

The particular order of the analysis algorithm for identification and categorization can vary (e.g. first barcode sequences B_{T}, then the barcode sequences B_{P}, then the barcode sequences B_{S}, then the UMI sequence; or barcode sequences B_{S}, then the barcode sequences B_{T}, then the barcode sequences B_{P}, then the UMI sequence; or first barcode sequences B_{P}, then the barcode sequences B_{S}, then the barcode sequences B_{T}, then the UMI sequence; or first UMI sequence, then barcode sequences B_{S}, then the barcode sequences B_{T}, then the barcode sequences B_{P}). According to one embodiment, the analysis algorithm may be repeated multiple times. According to one embodiment, the algorithm may be repeated as many times as required until the concentration/number of the biomolecules of interest for the time points for the positions, preferably until the concentration/number of all biomolecules of interest for all time points for all positions, is determined.

Other processing steps may also be performed in frame of the present method. Such processes include but are not limited to washing, purification, extraction, separation, centrifugation, sedimentation, etc. According to one embodiment, after step c) or d) the one or more cells can be extracted in an additional step, wherein the cell can be analyzed by sequential analysis means, including NGS sequencing of the genome or selected genes.

Suitable and preferred embodiments for the cell-laden matrix are disclosed in PCT/EP2020/056975, herein incorporated by reference. The matrix may comprise polymers and/or precursor molecule, preferably in a predominantly crosslinked form, which have been disclosed in PCT/EP2018/074527, in particular, polymers and/or precursor molecules disclosed in claims 101 to 155, which are herein incorporated by reference. In a preferred embodiment, the matrix comprises a hydrogel. The hydrogel may be a hydrogel as disclosed in PCT/EP2018/074527, in particular, hydrogels as disclosed in claims 1 to 51 and 72, which are herein incorporated by reference. PCT/EP2018/074527 further discloses methods for producing a hydrogel in claims 52 to 71, which are herein incorporated by reference. Furthermore, a kit for producing a hydrogel is disclosed in PCT/EP2018/074527 in claims 99 and 100, which are also herein incorporated by reference.

According to a preferred embodiment, the matrix is three-dimensional. According to one embodiment, three-dimensional may be understood as providing an environment that can be sensed spatially. For instance, a cell may sense a three-dimensional matrix around itself and not only at one side of the cell, which would be the case for planar matrixes. However, according to one embodiment of the present disclosure, three dimensional may also be understood as providing a quasi-planar environment to the cells at which cells are for instance at the border of a three dimensional matrix, wherein cells encounter a planar or curved surface at one side and a three-dimensional matrix at the other side.

According to another embodiment, further features may be provided by the three dimensional matrix, for instance by addition of further structural features such as topography, mechanical strain and shear stress into the matrix (which sometimes in the art is described as a fourth dimension but is here encompassed by the term “three-dimensional”). Furthermore, the matrix may be time- or signal-responsive, which may be understood as another dimension in the art. According to one embodiment, degradative molecules are added in order to degrade the matrix. For instance, if the matrix is crosslinked by hybridization of (complementary) molecules forming hydrogen-bridges (e.g. DNA-DNA-hybridization; PNA-PNA-hybridization; PNA-DNA-hybridization, etc.), degradative molecules may be added that interrupt the hybridization crosslinks. Thereby, the crosslinks may be released, whereupon the matrix can degrade to be capable of releasing the one or more cells.

The released cell might be further analyzed in regard to its genome and/or transcriptome by bimolecular techniques. The degradation of the hydrogel and the recovery of the cells provide the possibility to link the phenotype of the cell to the underlying genotype. According to one embodiment, methods for degrading a hydrogel matrix which have been described in PCT/EP2018/074527, in particular in claims 84 to 96 are applicable in conjunction with the present disclosure and are herein incorporated by reference. According to one embodiment, degradative molecules are secreted by encapsulated cells in order to remodel the surrounding matrix. For instance, if the matrix comprises matrix metalloproteinase target sites, cell secreted matrix metalloproteinases (MMP) degrade the cross-linked matrix. Secretion of degradable enzymes can enable cell motility and chemotaxis of the cells.

According to one embodiment, the matrix is a particle, preferably a spherical particle. The matrix is preferably a particle, having a shape selected from ellipsoidal, bead, spherical, droplet, elongated, rod, rectangular, and box. The matrix may have a regular shape, corresponding to an isometric particle or predominantly isometric particle. According to another embodiment, the matrix may have an irregular shape, corresponding to an anisometric shape.

According to one embodiment, the matrix diameter may be ≤1000 μm, such as ≤800 μm, ≤600 μm or ≤400 μm, preferably ≤200 μm. The matrix may have a diameter selected from a range of 5 μm to 1000 μm, and 10 μm to 500 μm, preferably selected from a range of 10 μm to 200 μm, 20 to 150 μm, and 50 to 100 μm. According to one embodiment, the matrix has a diameter of 80 μm. According to a preferred embodiment, the diameter of the matrix is adjusted to the size of the compartment, enabling transfer inside and out of the compartment. Moreover, the diameter of the matrix may be adjusted to the size of a microfabricated geometry for the immobilization of one or more matrices. The diameter may be considered as the longest axis of the matrix.

The cell-laden matrix may comprise one or more cells. The at least one cell may be selected from a prokaryotic and/or an eukaryotic cell. The at least one cell may be selected from the groups consisting of bacteria, archaea, plants, animals, fungi, slime moulds, protozoa, and algae. According to a preferred embodiment, the one or more cells may be selected from animal cells, preferably human cells. According to one embodiment, the at least one cell may be selected from cell culture cell lines. According to another embodiment, the one or more cells may be selected from the group consisting of stem cells, bone cells, blood cells, muscle cells, fat cells, skin cells, nerve cells, endothelial cells, sex cells, pancreatic cells, and cancer cells. According to another embodiment, the at least one cell may be derived from cells of the nervous system, the immune system, the urinary system, the respiratory system, the hepatopancreatic-biliary system, the gastrointestinal system, the skin system, the cardiovascular system, developmental biology (including stem cells), pediatrics, organoids, and model organisms. According to another embodiment, the at least one cell may be derived from one or more of blood and immune system cells, including erythrocytes, megakaryocytes, platelets, monocytes, connective tissue macrophages, epidermal Langerhans cells, osteoclast (in bone), dendritic cells, microglial cells, neutrophil granulocytes, eosinophil granulocytes, basophil granulocytes, hybridoma cells, mast cells, helper T cells, suppressor T cells, cytotoxic T cells, natural killer T cells, B cells, natural killer cells, reticulocytes, hematopoietic stem cells, and committed progenitors for the blood and immune system. According to one embodiment, the at least one cell may be derived from one or more of myoloid-derived suppressor cells type M (M-MDSC, tumor-supporting M2 macrophages, CAR-T cells, CAF (cyclophosphamide, doxorubicin, and fluorouracil)-treated cancer cells, cancer cells from residual tumors, cancer cells from relapsed tumors, cells isolated by biopsy, tumor initiating cells (TICs) and cancer stem cells, and tumor infiltrating lymphocytes (TILs).

The cell-laden matrix may comprise one cell. The cell-laden matrix may comprise more than one cell. According to another embodiment, the cell-laden matrix comprises a colony of cells. Preferably, a colony of cells can be located inside the three-dimensional matrix. According to another embodiment, the cell number changes throughout performing the method. For instance, the cell number increases over the course of cultivation, decreases over the course of cultivation or remains constant over the course of cultivation. A colony of cells may be formed by proliferation of one or more cells, wherein preferably cells proliferate inside the three-dimensional matrix. In another embodiment, the cell-laden matrix comprises at least two different types of cells that interact. In particular, the cell-laden matrix may comprise two different types of cells that interact. Alternatively, the cell-laden matrix may comprise three different types of cells that interact or four different types of cells that interact of five different types of cells that interact, or more than five different types of cells that interact.

According to another embodiment, more than one cell-laden matrix is provided. According to such an embodiment, the cell-laden matrices may be provided such, that at least two cells that interact with each other are analyzed and wherein each cell is located inside a three-dimensional matrix. Preferably, the cell-laden matrices are then located in close proximity to each other. According to an embodiment where more than one cell-laden matrix is provided, the cell-laden matrices may comprise one or more cells, wherein the cell number may be the same or different between the provided cell-laden matrices. The cell number may change throughout performing the method of the present disclosure. For instance, the cell number may increase throughout performing the method of the present disclosure, wherein the increase may be equal or different for different provided cell-laden matrices. When providing more than one cell-laden matrix, the type of cells present in per cell-laden matrix may be the same or different. For instance, immune cells may be provided in one cell-laden matrix and cancer cells in another cell-laden matrix, allowing to study the interaction of both types of cells.

According to one embodiment, migration between cells might be studied for secreted chemokines to combine information on secreted biomolecules with phenotypic function (e.g. migration of T-cell through matrix towards cancer cell (verifies that t-cell detects corresponding cytokine) and subsequent killing of cancer cell (verifies successful TCR binding as well as efficient killing of cancer cell). In addition, matrix might contain MMP (matrix metalloproteinase) sites for verification of matrix remodelling performed by efficient T-cells.

According to one embodiment, the method is not only useful for the analysis of the released molecules of single cells (or cell colonies) that are in proximity, preferably close proximity, but also for the analysis of chemoattractant-based cell-cell interaction between to different cell types (e.g. an immune cell and a cancer cell). To this end, chemo-attraction, migration and phenotypic interactions between cells positioned in two separated cell-laden matrices might be studied and linked to released biomolecules of interest (e.g. secreted chemokines). This enables identification of defined combinations of secreted biomolecules of interest with a distinct phenotypic function. For instance, the successful migration of T-cells through the matrix towards cancer cell located in a different cell matrix verifies that T-cells detect cancer cell-derived cytokine and/or chemokines and subsequent kill cancer cells by TCR recognition.

According to one embodiment, the method is not only useful for the analysis of chemoattractant-based cell-cell interactions but also for the analysis of direct cell-cell interactions. To this end, cells of the same or different type can be co-encapsulated within one cell-laden matrix (e.g. hydrogel matrix) and optionally brought into direct contact by cell-centring method disclosed in (PCT/EP2018/074526). Subsequently, the time-lapse secretion profile can be monitored as according to the present disclosure. For instance, an immune cell and a cancer cell can be co-encapsulated within one cell-laden matrix (e.g. hydrogel matrix) and the time-lapse secretion profile can subsequently be monitored as according to the present disclosure.

In one embodiment one single cell of a specific cell type is encapsulated within a matrix and subsequently positioned within a (microfabricated) compartment comprising at least one positioning mean. The analysis of released biomolecules of interest is performed as disclosed by positioning a capture matrix in close proximity to the cell-laden matrix. This has the advantage that the secretion profiles of isolated single cells can be generated which is in particular of importance for the characterization of heterogeneous cell populations and the identification of cells having unique and clinically relevant functions such as cancer stem cells. According to a preferred embodiment, methods for encapsulating at least one cell are performed as described in PCT/EP2018/074527, in claims 73 to 83, which are herein incorporated by reference.

In one embodiment two cells of different cell types are encapsulated within the same matrix and subsequently positioned within a (microfabricated) compartment. The Co-encapsulation can be performed by droplet formation using established techniques including corresponding sorting mechanism such as DEP-based sorting procedures (for instance, disclosed in PCT/EP2018/074526; Mazutis et al., 2013, Nature Protocols, 8, pages 870 to 891; Kleine-Bruggeney et al., 2019, Small, 15(5):e1804576). Subsequently a matrix (i.e. capture matrix) is positioned next to or in proximity to the cell-laden matrix within the same (microfabricated) compartment. This has the advantage that the isolated interaction of two different cell types can be analyzed. For example, one cell might secrete biomolecules that affect the neighboring cell thereby inducing certain cell responses. This is especially advantageous in the field of immuno-oncology. For example, the interaction between single cancer stem cells and single immune cells and the corresponding secreted biomolecules might give important insights on cell behavior and function. By the encapsulation of two cell types within one matrix, the distance between the two cell types can be minimized thereby increasing the chance that the cells get into direct contact. This is especially advantageous in processes where cell-cell contact is necessary for inducing a desired cell response.

In one embodiment two cells of different cell types are encapsulated within two separate matrices. Subsequently a capture matrix is positioned next to or in close proximity to the cell-laden matrices within the same (microfabricated) compartment. This has the advantage, that two cell types can be spatially separated in a controlled manner. Thus, it is possible to investigate paracrine signaling between two single cells provided in separate matrices (e.g. hydrogel matrices).

In one embodiment two or more cells of different cell types are encapsulated within separated matrices and subsequently positioned in proximity to each other within a (microfabricated) compartment. Subsequently a capture matrix is positioned next to or in proximity to the cell-laden matrices within the same (microfabricated) compartment. This has the advantage, that the interaction and signaling between multiple cell types can be studied.

In one exemplary embodiment, a cytotoxic T-cell, a macrophage and a tumor cell are encapsulated within separated matrices (preferably hydrogel matrices) and subsequently positioned in proximity of each other within a (microfabricated) compartment. Subsequently a capture matrix is positioned in proximity to the cell-laden matrices within the same (microfabricated) compartment. In frame of this embodiment, the migration of T-cells through the matrix towards cancer cell located in a different cell matrix verifies the detection of cancer cell-derived cytokine and/or chemokines by T-cells, the capability of matrix degradation and the ability to kill cancer cells by TCR recognition.

According to one embodiment, the released biomolecules of interest are released by one cell. According to another embodiment, the released biomolecules of interest are released by more than one cell. Furthermore, the number of cells that release the biomolecule of interest may change throughout performing the method according to the present disclosure. Alternatively, the number of cells may change when performing other methods or procedures. The mode of release may not be limiting according to the present disclosure. Cells may release biomolecules of interest via secretion vesicles. Cells may release biomolecules of interest via exocytosis.

The Biomolecules of Interest

According to the present disclosure, cells may release one or more biomolecules of interest. According to one embodiment, the method according to the first aspect allows to determine the profile of released biomolecules of interest. A profile of released biomolecules of interest may be understood as a time-dependent measurement of the absolute or relative amount of biomolecules of interest released and/or bound by the capture molecule. One or more biomolecules are preferably selected from the group comprising peptides, polypeptides and proteins (e.g. enzymes such as metalloproteases) and combinations thereof. Other biomolecules of interest may also be released and analyzed by the method according to the present disclosure. For instance, carbohydrates, nucleic acids, small organic molecules or lipids, glycopeptides and combinations thereof may be released (e.g. via secretion) and analyzed.

According to one embodiment, one or more types of biomolecules of interest are released. According to one embodiment, at least 1, at least 2, or at least 3 different types of biomolecules of interest are released, preferably at least 5, at least 6, at least 7, at least 8, or at least 9 different types of biomolecules of interest, more preferably 10 or more different types of biomolecules of interest are released and analyzed. According to one embodiment, one type of biomolecules is released and analyzed.

According to a preferred embodiment, the release of the one or more biomolecules of interest takes place by secretion of biomolecules. According to a particular embodiment, all biomolecules of interest that are released by secretion.

According to a preferred embodiment, the biomolecules of interest may be selected from cytokines. Moreover, one or more biomolecules of interest may be selected from the group consisting of cytokines, chemokines, interferons (INF), interleukins (IL), lymphokines, and tumor necrosis factor (TNF). According to another embodiment, the one or more biomolecules of interest are selected from the group consisting of interleukins (ILs), including IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36α, IL-36β, IL-36γ, IL-37, IL-1Ra, IL-36Ra and IL-38; interferons (INFs), including type I IFNs (such as IFN-α (further classified into 13 different subtypes such as IFN-α1, -α2, -α4, -α5, -α6, -α7, -α8, -α10, -α13, -α14, -α16, -α17 and -α21), and IFN-β, IFN-δ, IFN-ε, IFN-ζ, IFN-κ, IFN-v, IFN-τ, IFN-ω), type II IFN (such as IFN-γ) and type III IFNs (such as IFN-A1 and IFN-λ2/3); tumor necrosis factors (TNF), such as TNF-α, TNF-β, CD40 ligand (CD40L), Fas ligand (FasL), TNF-related apoptosis inducing ligand (TRAIL), and LIGHT; chemokines, including CCL1, CCL2, CCL3, CCL4, CCL5, CCL6, CCL7, CCL8, CCL9/CCL10, CCL11, CCL12, CL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, XCL1, XCL2, CX3C, and CX3CL1; other cytokines, such perforin, granzyme, MCP-1, MCP-2, MCP-3. Rantes, IP-10. Osteopontin, MIP-1a, MIP-1b, MIP-2, MIP-3a, MIP-5, VEGF, IGF, G-CSF, GM-CSF, Eotaxin, PDGF, Leptin, and Flt-3; and/or combinations thereof.

According to one embodiment, the one or more biomolecules of interest are selected independently for each time-point. For instance, in the beginning of the experiment growth factors such as EGF and VEGF are analyzed, in the middle of the experiment, chemokines such as CCL2 and CCL5 are analyzed and in the end of the experiment interleukins such as IL-6 and IL-10 are analyzed.

According to one embodiment, the capture matrix may be provided by a hydrogel, wherein the hydrogel can have one or more of the features described above in conjunction with the cell-laden matrix provided by a hydrogel. Furthermore, the capture matrix may be provided by a hydrogel, comprising a molecule, which is at least partly soluble in aqueous solutions and can be derived from one or more of the molecules described above in conjunction with the cell-laden matrix provided by a hydrogel. According to another embodiment, the capture matrix may be provided by a hydrogel comprising one or more polymers, especially a as building or for hydrogel formation, as described above. Suitable capture matrixes are described in PCT/EP2020/056975, herein incorporated by reference.

In a preferable embodiment, the capture matrix comprises a hydrogel, which preferably has a spherical shape. In a preferred embodiment, wherein the matrix comprises a hydrogel, a polymer or pre-polymer is chosen by one skilled person in the art from at least one of the polymers, pre-polymers or precursors described above in conjunction with the cell-laden matrix. The capture matrix may comprise polymers and/or precursor molecule, which have been disclosed in PCT/EP2018/074527, in particular, polymers and/or precursor molecules disclosed in claims 101 to 155, which are herein incorporated by reference. In a preferred embodiment, the capture matrix comprises a hydrogel. The hydrogel may be a hydrogel as disclosed in PCT/EP2018/074527, in particular, hydrogels as disclosed in claims 1 to 51 and 72, which are herein incorporated by reference. PCT/EP2018/074527 further discloses methods for producing a hydrogel in claims 52 to 71, which are herein incorporated by reference. Furthermore, a kit for producing a hydrogel is disclosed in PCT/EP2018/074527 in claims 99 and 100, which are herein incorporated by reference.

In the preferred embodiment, the matrix comprises a hydrogel, wherein the hydrogel comprises a polymer or pre-polymer (preferably predominantly in a crosslinked state) which is selected from the group comprising poly(lactic acid) (PLA), polyglycolide (PGA), copolymers of PLA and PGA (PLGA), poly(vinyl alcohol) (PVA), polyethylene glycol (PEG), poly(ethylene oxide), poly(ethylene oxide)-co-poly(propylene oxide) block copolymers (poloxamers, meroxapols), poloxamines, polyanhydrides, polyorthoesters, poly(hydroxyl acids), polydioxanones, polycarbonates, polyaminocarbonates, poly(vinyl pyrrolidone), poly(ethyl oxazoline), carboxymethyl cellulose, hydroxyalkylated celluloses such as hydroxyethyl cellulose and methylhydroxypropyl cellulose, and natural polymers such as nucleic acids, polypeptides, polysaccharides or carbohydrates such as polysucrose, hyaluranic acid, dextran and similar derivatives thereof, heparan sulfate, chondroitin sulfate, heparin, or alginate, and proteins including without limitation gelatin, collagen, albumin, or ovalbumin, or copolymers, or blends thereof. In particularly preferred embodiments, the monomers can be selected from polyactic acid (PLA), poly(vinyl alcohol) (PVA), polyethylene glycol (PEG), polyoxazoline (POx) and polyacrylamide (PAM).

According to a preferred embodiment, the capture matrix can be three-dimensional. As described above, other features may also be incorporated into the capture matrix and may be applicable in view of the present disclosure.

Moreover, the capture matrix may be a particle, preferably a spherical particle. Other shapes are also applicable for the capture matrix, including those described above in conjunction with the cell-laden matrix.

The capture matrix may have a diameter selected from a range of 5 μm to 1000 μm, such as 10 μm to 500 μm, preferably selected from a range of 10 μm to 200 μm, 20 to 150 μm, and 50 to 100 μm. Further characteristics in respect to the diameter have been described above for the cell-laden matrix and may also be applicable for the capture matrix.

In one embodiment at least two capture matrices comprising different types of capture molecules (e.g. antibodies) are subsequently positioned next to or in proximity to the cell-laden matrix or matrices within the same (microfabricated) compartment.

In another embodiment the capture matrix comprises one or more types of capture molecules, in particular antibodies. The capture matrix can be selected from polymer particles (e.g. beads), magnetic particles, hydrogel spheres/matrices/beads, or resins or combinations thereof.

In another embodiment, at least one capture particle is encapsulated within a matrix, preferably a hydrogel matrix. The at least one capture particle comprises one or more types of capture molecules and can be selected by the person skilled in the art from the group consisting of polymer particles (e.g. polystyrene beads), magnetic particles, hydrogel spheres/matrices/beads, resins or capture matrices or combinations thereof. The particle diameter may be ≤200 μm, such as ≤100 μm or ≤80 μm, preferably ≤30 μm. The at least one capture particle may have a diameter selected from a range of 0.1 μm to 100 μm, and 1 μm to 50 μm, preferably selected from a range of 0.1 μm to 80 μm, 0.5 to 50 μm, and 1 to 30 μm. The capture particle is preferable smaller in diameter than the matrix encapsulating said at least one capture particle. The polymers for the hydrogel matrix can be selected from polyactic acid) (PLA), poly(vinyl alcohol) (PVA), polyethylene glycol (PEG) and polyoxazoline (POx), polyacrylamide (PMA) and agarose.

As disclosed herein, the capture matrix may be contacted with the cell-laden matrix in different orders for binding the released one or more biomolecules of interest.

The Capture Molecules

According to a preferred embodiment, the capture matrix comprises one or more types of capture molecules. In particular, the one or more types of capture molecules may be bound to the capture matrix. They are bound such, that capture molecules are capable of binding biomolecules of interest, e.g. by providing a capture matrix that allows diffusion of the biomolecule of interest into the capture matrix to the capture molecules, by binding the capture molecules such that it does not prevent the capture molecules from binding to the biomolecules of interest. In an exemplary embodiment, one or more types of capture molecules may be incorporated by reaction(s) based on:
covalent bond formation chosen from the group consisting of:
   enzymatically catalyzed reactions
      transglutaminase factor XIIIa
   not-enzymatically catalysed
      click chemistry
      photo-catalyzed
   uncatalyzed reactions
      Copper-free highly selective click chemistry
      Michael-type addition
      Diels-Alder conjugation
non-covalent bond formation:
   Hydrogen bonds formed by:
      Nucleic acids
   Hydrophobic interactions
   Van-der-Waals
   Electrostatic interactions

According to one embodiment, incorporation of one or more types of capture molecules into said capture matrix (e.g. an oxazoline-based hydrogel matrix) is implemented by peptide nucleic acids. PNA oligomers may be incorporated by amide bond formation between the NHS-ester from the hydrogel precursor molecule and the primary amine of a PNA oligomer. The capture molecule may be fused to a complementary PNA oligomer. The fusion product may then be immobilized by hydrogen bond formation between the two PNA oligomers. The capture molecule can be removed by addition of a molar excess of complementary PNA oligomers. The complementary PNA oligomers can compete with the PNA/capture fusion product. Alternatively, the one or more types of capture molecule can be fused to a complementary modified PNA oligomer. The modification may comprise a photo-cleavable linker between two PNA molecules. After hydrogen bond formation between the two PNA oligomers, the capture molecule can be easily removed by UV irradiation. In both cases the capture molecule may comprise or consist of an antibody, a small molecule, an antigen, a protein binding domain, a nucleic acid, a polysaccharide or an aptamer.

The attachment of the capture molecule, in particular a capture antibody to the capture matrix can be performed by any reaction well known by the person skilled in the art, including but not limited to ligation chemistry such as Diels-Adler reaction, Michael addition, Staudinger ligation, affinity-tags, Biotin-Avidin and native chemical ligation.

According to a preferred embodiment, the one or more types of capture molecules are attached to the capture matrix such that the capture molecules are not released throughout steps a) to c), preferably steps a) to d). In the particular embodiment, wherein the one or more types of capture molecules are attached via hybridization of PNAs, the PNA sequence hybridization is adjusted to not result in dehybridization over any of the steps a) to c) and step d) (aa), wherein before amplification (e.g. step d) (bb)) preferably, the extended barcode oligonucleotide is released from the one or more types of detection molecules and the capture matrix is removed.

According to one embodiment, the one or more types of capture molecules are selected from the group consisting of proteins, peptides, nucleic acids, carbohydrates, lipids, polymers, and small organic molecules. According to one embodiment, the one or more types of capture molecules are selected from the group consisting of antibodies, antibody fragments, hybrid antibodies, recombinant antibodies, single-domain antibodies (nanobodies), recombinant proteins comprising at least a portion of an antibody, chimeric antibodies, humanized antibodies, multiparatopic antibodies, multispecific antibodies, fusion proteins, aptamers, DNA aptamers, RNA aptamers, peptide aptamers, receptors, receptor fragments, non-antibody protein scaffolds comprising a molecular recognition moiety—including DARPins (Designed Ankyrin Repeat Proteins), Repebodies, Anticalins, Fibronectins, Affibodies, engineered Kunitz domains—Affirmer proteins, Adhiron proteins, lipocalins, lipid derivatives, phospholipids, fatty acids, triglycerides, glycerolipids, glycerophospholipids, sphingolipids, saccharolipids, polyketides, cationic lipids, cationic polymers, poly(ethylene) glycols, spermines, spermine derivatives or analogues, poly-lysines, poly-lysine derivatives or analogues, polyethyleneimines, diethylaminoethyl (DEAE)-dextrans, cholesterols, sterol moieties, cationic molecules, hydrophobic molecules and an amphiphilic molecules, preferably selected from the group consisting of antibodies, antibody fragments, and aptamers. According to a preferred embodiment, the one or more types of capture molecules are antibodies or antigen binding fragments thereof. According to one embodiment, an antibody is used as a type of capture molecule, wherein the antibody specifically binds a first biomolecule of interest at a first epitope. This concept may also be used for further biomolecules of interest.

According to the present disclosure, the capture matrix can comprise one or more types of capture molecules, wherein each type of capture molecule is capable of specifically binding a biomolecule of interest. According to one embodiment, the capture matrix comprises at least 2, at least 3, at least 4, at least 5, at least 6, at least 7, at least 8, at least 9, or at least 10 different types of capture molecules, wherein each capture molecule is capable of specifically binding a biomolecule of interest and wherein each type of capture molecule is capable of binding to a different type of biomolecule. It is within the scope of the present disclosure that a type of capture molecule is provided by different kinds of capture molecules (e.g. antibody and antibody fragment) which bind to the same type of biomolecule of interest (e.g. at the same or a different epitope). According to a preferred embodiment, one type of capture molecules comprises multiple identical capture molecules. Preferably multiple capture molecules of each type are bound to the capture matrix. This ensures efficient capture of released biomolecules of interest.

In one embodiment, the capture matrix comprising capture molecules (e.g. immobilized detection antibodies) can be provided (e.g. by positioning positioned the capture matrix in the compartment) in a delayed manner to improve the natural phenotype of cells such as autocrine and paracrine signaling. The release of various cytokines can dependent on each other (e.g. IL-2 and IL-5). If IL-2 is reduced, for instance, due to capture by binding to the capture molecules, no IL-5 is secreted resulting in false negative results. Such an effect can be avoided by cultivation of cells without presence of capture molecules for a prolonged period and addition of the capture matrix with capture molecules afterwards. To prevent loss of biomolecules of interest during the positioning of the capture matrix a biphasic compartment generation (for example as disclosed in PCT/EP2018/074526) can be used. Thus, the cell-laden matrix located within an aqueous phase is first positioned within a compartment of the cell culture device (preferably a microfabricated cell culture device). Afterwards, the aqueous phase is exchanged by an oil phase (such as fluorinated oil (e.g. HFE-7500)) and the cell-laden matrix is incubated for a defined period. Then, a capture matrix located within an aqueous droplet is delivered via the oil phase to the position of the cell-laden matrix. This has the advantage that the secreted molecules are not washed away as soon as the capture matrix is positioned next to the cell-laden matrix.

The detection molecules are described in detail in the claims and figures.

The same or different number of types of detection molecules and types of capture molecules may be applied. Such an embodiment may be found useful in case a type of capture molecules is capable of binding more than one type of biomolecule of interest (e.g. of the biomolecules of interest have a similar or equal recognition moiety). Afterwards, for detection, it may be useful to differentiate between the biomolecules of interest and thus provide a greater number of types of detection molecules than the number of types of capture molecules. Vice versa, it may also be useful to provide a greater number of types of capture molecules than detection molecules (e.g. in case the detection molecules bind a number of similar biomolecules of interest, where a differentiation between the different types of biomolecules (e.g. subspecies of biomolecule types) is not required and/or desired).

The Device Comprising the Cell-Laden Matrix and the Capture Matrix

According to a preferred embodiment, the cell-laden matrix and the capture matrix are provided in proximity to each other within an isolated compartment of a device. According to another embodiment, a plurality of cell-laden matrices and capture matrices are provided in a cell culture device comprising a plurality of compartments, in particular an isolated compartment or compartments that can be isolated from each other, wherein a cell-laden matrix and a capture matrix are provided within a compartment, in particular an isolated compartment, of the cell culture device. According to a preferred embodiment, a device which can be utilized for performing the method of the present disclosure corresponds to a device disclosed in PCT/EP2018/074526 in claims 40 to 73 and these are herein incorporated by reference. Other devices are also described elsewhere herein and in the examples.

According to a preferred embodiment, the method is performed utilizing a cell culture device, preferably a microfabricated cell culture device. The device may comprise a compartment for accommodating one or more cell-laden matrices. Preferably, the device comprises an array of compartments for accommodating cell-laden matrices. Moreover, the device may comprise a fluid reservoir and fluid channels for supplying fluid to the compartment. The device preferably further comprises means to switch the compartment between a closed state and an open state, wherein the closed state corresponds to a state at which fluid that is present in the compartment is in no contact with fluid not present in the compartment and wherein the open state corresponds to a state at which fluid that is present in the compartment is in contact with fluid not present in the compartment. The closed state of the compartment may correspond to an isolated compartment, wherein an at least partially closed system may be provided.

The device may comprise one or more microfabricated valves, wherein preferably the one or more microfabricated valves are capable of switching the compartment between an open and closed state. According to a preferred embodiment, the microfabricated valve comprises a first channel, a second channel, a connection channel connecting the first channel and the second channel, a valve portion arranged within the connection channel, wherein the valve portion is adapted to selectively open and close the connection channel. Moreover, the microfabricated valve may comprise at least three layers, wherein a first channel is located within a first layer; a second channel is located within a third layer; a valve portion is located within a second layer; the second layer is arranged between the first and the third layer.

The device may comprise microfabricated geometries and means for handling and processing of particles, in particular hydrogel matrices. Said handling and processing includes for example geometries and means for the positioning of particles at a pre-defined location, the storage of said particles at the position for a pre-defined period, the controlled retrieval of positioned particles and the transfer of retrieved particles to another pre-defined location.

Furthermore, the device may comprise a microfabricated valve, wherein a first channel comprises a positioning mean suitable for positioning one or more cell-laden matrices and/or capture matrices being contained in a fluid which flows through the first channel, wherein the positioning mean is arranged within the first channel in such a way that a fluid flow can be reduced by the positioning means, in particular, the positioning means narrows the cross section of the channel. According to one embodiment, the device comprises a second channel comprising a positioning mean suitable for positioning one or more cell-laden matrices and/or capture matrices being contained in a fluid which flows through the second channel, wherein the positioning means is arranged within the second channel in such a way that a fluid flow can be reduced by the positioning means, in particular, the positioning means narrows the cross section of the channel. According to a preferred embodiment, the device comprises one or more compartments for accommodating one or more cell-laden matrices and/or capture matrices, wherein a positioning mean is present suitable for positioning the one or more cell-laden matrices and/or capture matrices inside the compartment.

Positioning and Removal of Matrices

The first advantage is that matrices with defined characteristics (such as size, composition (e.g. immobilization of compounds or cells)) can be positioned on said array of the cell culture device in a programmable manner. For example, if said array has n×m microfabricated individualizable compartment (n representing the number of rows and m representing the number of columns), a defined number of particles, in particular spherical hydrogel matrices with defined characteristics can be positioned in each of the n×m microfabricated compartments. Thus, one microfabricated compartment might contain one or more matrices that might contain no, single or multiple cells of the same or of different type or that might contain capture molecules. For example, a first matrix that contains one single cell of cell type 1 might be positioned next to a matrix that contains one or more types of capture molecules in one microfabricated compartment.

A second advantage in comparison to the prior art is that said immobilized matrices can be removed in a defined way from said array at any time-point and from any position and said removed matrices can subsequently be transferred into another format such as a well plate or similar format. In addition, removal of said matrices does not affect matrix integrity (e.g. hydrogel integrity) and thus results in a higher cell viability as well as in a maintenance of any information (such as bound molecules) that might be associated with the matrices. For example, if a first matrix is located within a microfabricated compartment at position (n, m) and a second matrix is located within close proximity to the first matrix or is in direct contact with the first matrix, the second matrix might be removed first while the first matrix stays within the microfabricated compartment. Afterwards, the first matrix might be removed in a second step. This can also be done for more than two matrices.

A further advantage of the present disclosure is that matrices located within different microfabricated compartments can be removed simultaneously. For example, a first matrix located within a microfabricated compartment (n1, m1) might be removed at the same time at which a second matrix located within a microfabricated compartment (n2, m2) is removed. This can also be done for more than two matrices located at more than two different positions. Thus, the advantage is a significant reduction of time needed for removing said matrices and transferring them into another format suitable for a corresponding downstream analysis.

Details are also described in PCT/EP2020/056975, herein incorporated by reference.

Controllable Fluid Perfusion

A further advantage of the cell culture device is that microfabricated compartments can be individually perfused with a fluid. For example, cells located in matrices (i.e. cell-laden matrices) positioned in an array of said cell culture device can be continuously or stepwise perfused with fresh cultivation medium resulting in a removal of cellular waste products and supply with fresh nutrients. Thus, cells can be cultivated within n×m microfabricated compartments for an extended period as new nutrients can be supplied continuously whereas all microfabricated compartments might have the same culture conditions.

The Microfabricated Valve

According to one embodiment, the present disclosure relates to microfabricated structures and methods for the control of fluid flows within said cell culture device using a microfabricated (elastomer) valve. According to a preferred embodiment, the cell culture device comprises a microfabricated valve as disclosed in PCT/EP2018/074526 in claims 1 to 39, herein incorporated by reference. One of the main advantages of said microfabricated valve is that it can be used for performing and improving the most critical and important processes used in microfluidic devices as well as in the field of microdroplet microfluidics and in particular, for the generation of the disclosed array. In particular, these processes include control of fluid flows, fluid pumping and fluid mixing in microfluidic devices as well as the formation of droplets, formation of encapsulation, in particular single-cell encapsulations, co-encapsulation, droplet mixing, the formation of (hydrogel) matrices and droplet de-mulsification in terms of microdroplet-based microfluidics. The main advantage of said microfabricated elastomer valve is the low actuation pressure (≤100 mbar) that is needed for its actuation as well as the nominal diameter that is suitable for the transport of larger matrices. Another advantage of the elastomer valve is that it can be fabricated in a cost-effective and simple manner using standard multilayer lithography methods. In a first embodiment said microfabricated structure for flow control comprises a first microfabricated layer with recesses comprising a first microfabricated channel which is defined as “first flow channel” and a second microfabricated layer that has a recess which connects the first microfabricated channel with the space above the second microfabricated channel. This recess is defined as “connection channel”. The connection channel is separated by a second recess of the second microfabricated layer by a thin elastomeric membrane with a thickness between 1 μm and 80 μm. The first flow channel might contain a first fluid and the space above the second microfabricated layer might contain a second fluid of the same or of different type. The recess within the second microfabricated layer that is separated by an elastomeric membrane from the connection channel is here defined as “actuation channel”.

Channels

The term “channel” requires at least any cavity which is adapted to accommodate a fluid. In an embodiment the channel may constitute a part of a conduct for conducting a stream of fluid. A channel may be a formed by a fluid conduct; a channel may be formed by a reservoir. Such a reservoir may be closed or may be open with a connection to the atmosphere. In an embodiment the channel may be a reservoir. For example, this reservoir may be closed except for the opening which connects it to another channel. Alternatively the reservoir may be open, for instance it may have an open upper end. In a one embodiment the second channel is a reservoir, in particular an open reservoir.

The Valve Action

Opening and closing of valve. In one embodiment, the actuation channel contains a fluid such as air or fluorinated oil (e.g. HFE-7500 (Novec)). Upon increasing the pressure in said actuation channel, a pressure difference between the connection channel and the actuation channel is generated. Thus, an actuation force is acting on the elastomeric membrane separating the connection channel and the actuation channel. This actuation force results in a bending of the membrane and a closing of the connection channel thereby separating the first flow channel from the space above the second microfabricated layer. After removing said pressure, the connection channel opens again due to the elastomeric characteristics of the used membrane. In a particular embodiment, the deflection distance of the membrane might be in the range of 1 μm to 100 μm. In another embodiment, the connection channel is not fully closed and thus the hydrodynamic resistance of the connection channel can be controlled in a defined manner by changing the applied pressure and thus the actuation force acting on the membrane. In one embodiment, the pressure might be varied between 0 mbar and 4000 mbar (absolute pressure) in steps of 1 mbar to adjust the hydrodynamic resistance of the connection channel. In particular embodiments the actuation force might be applied by using fluids (hereinafter also referred to as control fluid or actuating fluid) of the following type:
Gases such as air, nitrogen and argon
Liquids such as water, silicon oils, fluorinated oils and other oils
Solutions containing salts and/or polymers such as polyethylene glycol or glycerol
Ferromagnetic fluids
Hydrogels that are capable of swelling and shrinking upon application of a stimulus.

For example said stimulus might be one of the following types: temperature, ionic strength, electric field strength, magnetic field strength, pH value

In addition to applying an actuation force via a pressure-based actuation system, valve actuation might be performed by other actuation systems that might be of the following types: electrostatic, magnetic, electrolytic or electrokinetic.

Valves can be actuated by injecting gases (e.g., air, nitrogen, and argon), liquids (e.g., water, silicon oils and other oils), solutions containing salts and/or polymers (including but not limited to polyethylene glycol, glycerol and carbohydrates) and the like into the control channel, a process preferred to as “pressurizing” the control channel. In addition to elastomeric valves actuated by pressure-based actuation systems, monolithic valves with an elastomeric component and electrostatic, magnetic, electrolytic and electrokinetic actuation systems may be used. See, e.g., US 20020109114; US 20020127736, and U.S. Pat. No. 6,767,706.

In particular embodiments valves (including valves with dimensions as described above) do not completely block the flow channel lumen with the membrane is fully actuated by a control channel pressure of 30, 32, 34, 35, 38 or 40 psi.

Fluid injection. In another advantageous embodiment, the space above the second microfabricated layer is composed of a recess within a third microfabricated layer that is defined as “second flow channel”. The second flow channel might contain a fluid of type 2 and the first flow channel might contain a fluid of type 1 with fluid of type 2 and fluid of type 1 being miscible. A defined amount of the fluid of type 2 might be injected into the fluid of type 1 by applying a hydrodynamic pressure within the second flow channel that is larger than the hydrodynamic pressure in the first flow layer and by opening said elastomer valve for a defined time (e.g. 0.1 ms to 500 ms). The main advantage of using said microfabricated elastomer valve for injection of a fluid is the short opening and closing time that is needed due to the low actuation pressure resulting in a very fast valve operation. The opening time may be for example be 1, 2, 3, 4, 5 ms, s. or min. Methods for injecting a fluid have been described in PCT/EP2018/074526 in conjunction with claims 74 to 77 and 95 to 98 relating to methods for creating droplet, which are herein incorporated by reference. A droplet may comprise hydrogel particles, a hydrogel matrix, hydrogel beads, hardened and/or gelled and/or polymerized hydrogels or any other accumulated particles in particular are bonded to each other in a chemical or physical way (e.g. by surface tension), that keeps the particles together and delimits the accumulated particles from the environment, in particular a fluid surrounding the particles. According to a preferred embodiment, a droplet when injected comprises or consists of a liquid. In case the droplet comprises a liquid, the droplet predominantly consists of a liquid but further components can be present, for instance, as in a suspension (e.g. a micro- or nanoparticle suspension). After droplet injection, preferably one or more compound present in the predominantly liquid droplet react to form a matrix. Compounds which may be applied to form a matrix have been described above in conjunction with the cell-laden matrix and the capture matrix and it is here referred to those compounds. For instance, one or more of the polymers, pre-polymers, buildings blocks, precursor, monomers, etc. may be applied to generate a matrix after droplet formation. In one particular example, a precursor is dissolved and then injected to form a droplet. Over the course of transporting the droplet, the precursor may react, e.g. by polymerizing, to form a matrix. Various modes of matrix formation may be applicable in scope of the present disclose and have been described for instance in PCT/EP2018/074527.

Parallel actuation. In another advantageous embodiment, multiple microfabricated elastomeric valves might be actuated simultaneously which increases the process speed by parallelization. To this end, multiple microfabricated valves are located within the same actuation channel. If an actuation force is applied in said actuation channel, all microfabricated valves are closed at the same time. Each microfabricated valve might have a first and a second flow channel as described above which are separated from the first and second flow channels of the other microfabricated valves. Thus, different fluids located in the second flow channels might be injected simultaneously into different fluids located in the first flow channel. In another embodiment, all microfabricated valves are connected to the same second flow channel.

Details are also described in PCT/EP2020/056975, herein incorporated by reference.

Layers

In an especially preferred embodiment, the microfabricated valve comprises at least three layers, wherein the first channel is located within a first layer, the second channel is located within a third layer, the valve portion is located within a second layer and the second layer is arranged between the first and the third layer.

The use of three layers enables manufacturing of a vast number of different microfabricated valves. This increases the design variety and allows designing microfabricated valves according to different process requirements, like mixing of different fluids.

Moreover, this embodiment provides a vast number of possible valve designs and allows configuring the microfabricated valve according to the desired application.

Microfabricated Compartments

Microfabricated compartment for matrix immobilization and removal. In another aspect, the present disclosure relates to microfabricated structures and methods for the controlled positioning and sequential removal of matrices within microfabricated compartments.

According to a preferred embodiment, methods as disclosed in PCT/EP2018/074526 in claims 78 to 98 can be utilized in scope of the present disclosure. Hence, the said disclosure, in particular claims 78 to 98 are herein incorporated by reference. Details are also described in PCT/EP2020/056975, herein incorporated by reference.

In a first advantageous embodiment, microfabricated compartments located within said array might have at least one inlet and one outlet. A first microfabricated compartment at position (1,1) might be connected to a second microfabricated compartment (2,1). To this end, the outlet of microfabricated compartment (1,1) acts as an inlet for microfabricated compartment (2,1). The microfabricated compartment at position (2,1) might be connected to a third microfabricated compartment (3,1). Thus, all microfabricated compartments from one column n might be connected so that microfabricated compartment (n−1,1) is connected to microfabricated compartment (n,1). In addition, a microfabricated compartment positioned at (n,1) might be connected to a microfabricated compartment (1,2) which might be connected to a microfabricated compartment positioned at (2,2). This might be repeated so that all microfabricated compartments can be perfused simultaneously with the same fluid. The inlet of microfabricated compartment (1,1) might be connected to a reservoir for supply with different fluids. The outlet of the microfabricated compartment (n,m) might be connected to a collection reservoir. Thus, all connected microfabricated compartments might be perfused with the same fluid. For example, said perfusion fluid might be an aqueous phase containing nutrients or a suspension containing one or more matrices. The inlets and outlets of said microfabricated compartments might be closed by using an elastomer valve as described within the present disclosure. Microfabricated compartments might be first loaded with a fluid and then isolated from each other by closing said valves. Thus, a fluid volume located within microfabricated compartment (1,1) cannot be mixed with a fluid volume located within another microfabricated compartment (n,m). This has the advantage that the cell-cell communication between cells located within different microfabricated compartments might be prevented which is of importance as any secreted molecules from cells located within a first microfabricated compartment might influence the cell response of cells located within a second microfabricated compartment.

Positioning on Chip

Details are also described in PCT/EP2020/056975, herein incorporated by reference.

Sequential positioning. In another embodiment, said connected microfabricated compartments might be perfused with a solution containing one or more particles in particular hydrogel matrices. Said microfabricated compartments might contain a microfabricated geometry for the positioning of matrices in particular for hydrodynamic trapping of matrices. If a first microfabricated compartment does not contain any matrices, a first matrix entering said microfabricated compartment will likely be positioned within a microfabricated trapping geometry. The positioning of said first matrix might change the hydrodynamic resistance of the microfabricated compartment so that a second matrix that enters said microfabricated compartment moves into a bypass channel and afterwards enters a second microfabricated compartment. Said second matrix might be immobilized within the second microfabricated compartment. A third matrix might then bypass the first and the second microfabricated compartment, entering the third microfabricated compartment. Thus, matrices might be positioned in connected microfabricated compartments in a sequential manner—a first incoming matrix might be positioned within a first microfabricated compartment, a second incoming matrix might be positioned within a second microfabricated compartment and so on.

Defined positioning of matrices with different compositions. In another embodiment, matrices located within microfabricated compartments of said array might have different compositions. For example, a first matrix of type 1 might be generated by the on-demand formation and fusion of several droplets into one larger droplet and subsequent positioning of said droplet for cell/particle centering, hydrogel formation and demulsification as is described in the prior art. The matrix might be located within a microfluidic channel that is connected to a first microfabricated compartment. Thus, a pressure might be applied so that the matrix enters said microfabricated compartment and said matrix of type 1 might be positioned in said first microfabricated compartment. Said process might be repeated for the generation of a matrix of type 2 which is subsequently positioned within a second microfabricated compartment located next to said first microfabricated compartment. This process composed of matrix generation and immobilization might be repeated until all microfabricated compartments contain one matrix.

Positioning of two matrices within one microfabricated compartment. In another embodiment, said microfabricated compartments might have a microfabricated geometry for the positioning of two matrices of the same or of different type either in contact or in close proximity. To this end, a first microfabricated compartment might have a trapping geometry as well as a bypass channel. If a first matrix enters said first microfabricated compartment, the matrix moves into the trapping geometry as the main volume flow goes through said trapping geometry. A second matrix entering said first microfabricated geometry might enter the same trapping geometry as the hydrodynamic resistance of the bypass channel is larger than the hydrodynamic resistance of the trapping geometry containing one matrix. After trapping of two matrices the hydrodynamic resistance of said microfabricated trapping geometry increases and a third matrix moves into the bypass channel and afterwards to a second microfabricated compartment.

Positioning of three matrices within one microfabricated compartment. In another embodiment, said microfabricated compartments might have a microfabricated geometry for the positioning of three matrices of the same or of different type either in contact or in close proximity. To this end, a first microfabricated compartment might have a trapping geometry as well as a bypass channel. If a first matrix enters said first microfabricated compartment, the matrix moves into the trapping geometry as the main volume flow goes through said trapping geometry. A second matrix entering said first microfabricated geometry might enter the same trapping geometry as the hydrodynamic resistance of the bypass channel is larger than the hydrodynamic resistance of the trapping geometry containing one matrix. This is also true for a third matrix entering said first microfabricated compartment. After trapping of three matrices the hydrodynamic resistance of said microfabricated trapping geometry increases and a fourth matrix moves into the bypass channel and afterwards to a second microfabricated compartment.

Further configurations, wherein more than three matrices may be positioned or two or more matrices may be positioned within one compartment are also applicable in view of the present disclosure.

Reverse Flow Cherry Picking (RFCP)

According to one embodiment, the cell-laden matrix is preferably located inside a three-dimensional matrix and is releasably fixed by a positioning mean inside a compartment. Moreover, the cell-laden matrix and/or the capture matrix can be releasably fixed by a positioning mean inside a compartment, in particular within the same compartment. According to one embodiment, the cell-laden matrix and capture matrix are fixed by a positioning mean inside a compartment, wherein the positioning mean has one or more of the following characteristics:
it is capable of fixing the cell-laden matrix and the capture matrix next to each other, wherein optionally, the cell-laden matrix and the capture matrix may be in direct contact with each other or positioned with a distance between both matrices of less than 100 μm, 50 μm, 30 μm, 10 μm, 5 μm, or 1 μm; The cell-laden matrix and the capture matrix may contact each other at a single or multiple points, in particular they may share the same contact surface.
it is capable of fixing at least one cell-laden matrix and the capture matrix next to each other;
it is capable of fixing more than one cell-laden matrix, wherein the cell-laden matrices comprise either a single cell and/or a colony located inside a three-dimensional matrix, and the capture matrix next to the more than one cell-laden matrix; and/or
it is capable of fixing two cell-laden matrices, which are each located inside a three-dimensional matrix, and the capture matrix next to each other.
it is capable of fixing at least one cell-laden matrix and at least one capture matrix to each other.

According to one embodiment, the cell-laden matrix and the capture matrix can be fixed by a positioning mean inside a compartment, wherein the compartment accommodating the cell-laden matrix is different from the compartment accommodating the capture matrix and wherein both compartments can be switched to be either in fluid contact with each other or to be in no fluid contact with each other.

According to one embodiment, the compartment can have a valve arrangement adapted to provide a fluid passing through a positioning mean wherein the valve arrangement is adapted to selectively change the direction of fluid passing the location, in particular wherein a fluid is directed such urging the cell-laden matrix and/or the capture matrix into the positioning mean and a fluid in the second direction urging the cell-laden matrix and/or the capture matrix out of the positioning mean, and in particular fluid in the second direction delivering the cell-laden matrix and/or the capture matrix in direction of an exit section. Thereby, preferably, the cell-laden matrix and/or the capture matrix may be transported into a fixed position of the compartment, as well as transported out of a fixed position towards and exit section. The cell-laden matrix and/or the capture matrix may further be transported to other compartments to store and process the cell-laden matrix and/or the capture matrix. According to a preferred embodiment, the capture matrix can be introduced into and removed from a compartment in order to capture secreted biomolecules of interest for a tailorable amount of time. After the tailored amount of time has passed, the capture matrix can be removed from the compartment and another capture matrix can be added.

According to one embodiment, detection matrices can be obtained from the (isolated) compartments and be transferred to a separate device comprising a plurality of compartments, wherein each detection matrix is transferred to an isolated compartment of the device.

In a preferred embodiment, said device is a 96-well plate, a 384-well plate, a 1536-well plate.

Details are also described in PCT/EP2020/056975, herein incorporated by reference.

Further Embodiments of RFCP

Details are also described in PCT/EP2020/056975, herein incorporated by reference.

Removal of matrices from position (n,m). In one advantageous embodiment, matrices might be located within a microfabricated chamber at position (n, m) within said n×m array that enables the spatial immobilization of matrices as well as the transfer of said matrices into another format such as a 96-well plate at a desired time-point.

To this end, said microfabricated compartment might comprise a microfabricated geometry for the immobilization of matrices. In addition, said microfabricated compartment might contain at least two inlets and two outlets—a first inlet and a first outlet as well as a second inlet and a second outlet. The first inlet and the first outlet might be closed by using a first microfabricated valve as described previously. In addition, the second inlet and the second outlet might be closed using a second microfabricated valve as described previously as well. For the immobilization of matrices, said microfabricated compartment is perfused with fluid containing single or multiple matrices from the first inlet to the first outlet while the second inlet and the second outlet are closed. Afterwards, the first inlet and the first outlet might be closed and the microfabricated compartment might be perfused with a perfusion fluid from the second inlet to the second outlet.

Embodiments of said trapping geometry will be described in a following section of this disclosure. Said trapping geometry is connected to at least four microfluidic channels with defined hydrodynamic resistances, a first and a second microfluidic channel having a hydrodynamic resistance R₂ and R₃, respectively and a third and a fourth microfluidic channel with the hydrodynamic resistances R₄ and R₁, respectively (FIG. 11). The hydrodynamic resistances of the first and the second microfluidic channel (R₂ and R₃) might be increased by using microfabricated valves such as described previously (elastomer valve) with a first microfluidic valve v₁ (Vm2) for controlling the hydrodynamic resistance R₂ and a second microfluidic valve v₂ (Vn2) for controlling the hydrodynamic resistance R₃. The microfabricated structure comprising a microfabricated geometry for the immobilization of matrices might have the resistance R₀. The first microfluidic channel might be connected on one side with the fourth microfluidic channel as well as with the microfabricated geometry for matrix immobilization (defined here as node N₀₁₂) and on the other side with the third microfluidic channel (defined here as node N₂₄). In addition, the third microfluidic channel might be connected on the other side to the microfabricated geometry for matrix immobilization as well as to the second microfluidic channel (defined here as node N₀₃₄). The second microfluidic channel might be connected on the other side to the fourth microfluidic channel (defined here as node N₁₃) which might be connected to the first microfluidic channel and the microfabricated geometry for matrix immobilization (node N₀₁₂) (FIG. 11). The hydrodynamic pressure p₁ at the intersection of the first microfluidic channel and the third microfluidic channel (node N₂₄) might be higher than the hydrodynamic pressure p₂ at the intersection of the second and the fourth microfluidic channel (node N₁₃). The described hydrodynamic resistances, pressures and connections are analogous to an unbalanced Wheatstone bridge known from electronic circuits. A microfabricated geometry having said resistances and characteristics is here considered as a “reverse flow cherry picking (RFCP)” geometry. A matrix might be immobilized within said microfabricated geometry for matrix immobilization. A volume flow of a fluid from node N₀₁₂ to node N₀₃₄ might perfuse the microfabricated geometry for immobilization and an immobilized matrix might stay within its position. A volume flow of a fluid from node N₀₃₄ to node N₀₁₂ might result in a removal of said matrix from its position as the volume flow is reversed (this condition is defined here as “reverse flow” condition). An immobilized matrix might require a reverse flow with a critical flow rate of Q_{crit} to be removed. Thus, a reverse flow with a flow rate Qᵣₑᵥₑᵣₛₑ below Q_{crit} (Qᵣₑᵥₑᵣₛₑ<Q_{crit}) might not result in a removal of said matrix. In contrast, a reverse flow with a flow rate Qᵣₑᵥₑᵣₛₑ larger or equal than Q_{crit} might result in a removal of said immobilized matrix from its immobilization position. Depending on the actuation of the microfabricated valves v₁ (Vm2) and v₂ (Vn2) four different conditions might be distinguished:
1. Both valves are not actuated: In terms of this condition, the hydrodynamic resistances R₂ and R₃ are smaller than the hydrodynamic resistances R⁴ and R₁. The microfabricated geometry for the immobilization of matrices is mainly perfused from node N₀₁₂ to node N₀₃₄. Thus, an immobilized matrix stays within its position as the volume flow is not reversed.
2. Only valve v, (Vm2) is actuated while v₂ (Vn2) is not actuated: In terms of this condition, the resistance R₂ is increased and the main volume flow goes from node N₂₄ to node N₀₃₄ and from node N₀₃₄ to node N₁₃. If the microfabricated valve v₁ (Vm2) is not fully closed, the volume flow at the trapping position might go from N₀₁₂ to N₀₃₄ and the volume flow is not reversed. An immobilized matrix remains within its position. If the microfabricated valve v₁ (Vm2) is fully closed, a small volume flow might go from N₀₃₄ to N₀₁₂ with Qᵣₑᵥₑᵣₛₑ being smaller than Q_{crit}. Thus, an immobilized matrix remains within its position.
3. Only valve v₂ (Vn2) is actuated while v₁ (Vm2) is not actuated: In terms of this condition, the resistance R₃ is increased and the main volume flow goes from node N₂₄ to node N₀₁₂ and from node N₀₁₂ to node N₁₃. If the microfabricated valve v₂ (Vn2) is not fully closed, the volume flow at the trapping position might go from N₀₁₂ to N₀₃₄ and the volume flow is not reversed. An immobilized matrix remains within its position. If the microfabricated valve v₁ (Vm2) is fully closed, a small volume flow might go from N₀₃₄ to N₀₁₂ with Qᵣₑᵥₑᵣₛₑ being smaller than Q_{crit}. Thus, an immobilized matrix remains within its position.
4. Both valves v₁ (Vm2) and v₂ (Vn2) are actuated: In terms of this condition, the resistance R₂ as well as the resistance R³ are increased and the main volume flow goes from node N₂₄ to node N₀₃₄, from node N₀₃₄ to node N₀₁₂ and from node N₀₁₂ to node N₁₃. Thus, a reverse flow is generated at the trapping position that might have a flow rate of Qᵣₑᵥₑᵣₛₑ larger than Q_{crit}. Thus, an immobilized matrix is removed from its position and moves via node N₀₁₂ to node N₁₃.

In another embodiment, various types of objects may be positioned within a RFCP-geometry and retrieved as disclosed in the present disclosure. In a particular embodiment, said objects may be biological cells, such as prokaryotic and/or eukaryotic cells, in particular cells of the immune system, cells related to different types of cancer, cells of the nerve system, stem cells. In another advantageous embodiment, said objects may be cell aggregates, in particular embryonic bodies and or spheroids composed of different cell types. One of the main advantages of positioning cells within a RFCP-geometry is that cells might be first characterized when immobilized within a RFCP-geometry and subsequently sorted using the disclosed retrieval mechanism represented by a generation of a reverse flow.

In another embodiment, the advantage of positioning matrices containing cells within an RFCP-geometry is that single and/or multiple cells can be cultivated and observed for an extended time period in a highly defined microenvironment that is provided by the matrix. In another embodiment, matrices may contain biological compounds, in particular proteins, in particular antibodies, antibody-DNA conjugates, extracellular matrix proteins, growth factors, nucleic acids, in particular DNA, RNA, PNA, LNA, lipids, cytokines, chemokines, aptamers as well as metabolic compounds, chemical compounds, in particular small molecules, in particular drugs, molecules linked via photocleavable spacer/linker, nanostructures, in particular gold nanoparticles, growth promoting substance, inorganic substances, isotopes, chemical elements.

The advantage of the RFCP geometry is that immobilized matrices might be trapped and removed in a reversible manner by controlling the corresponding valve positions. In addition, as the removal process is based on a reverse flow, the removal process is cell compatible and very gentle in comparison to other methods (such as the use of a higher temperatures for generation of bubbles or for the degradation of said matrices) which is critical for handling single cells or small cell populations. In addition, the removal process maintains the integrity of immobilized matrices which is critical if said matrices store any information (e.g. secreted analytes bound to probes immobilized within said matrices) that might be accessed at later stage.

Removing a matrix from position n,m. In another advantageous embodiment, multiple RFCP geometries might be arranged within an n×m array whereas a matrix located at position (n,m) might be specifically removed from said array with a dramatic reduction in the number of actuators needed for removing said matrix. To this end, the microfabricated valves v₁ from all RFCP geometries located in row n might be actuated by a first actuator An and the microfabricated valves v₂ from RFCP geometries located in column m might be actuated by a second actuator Aₘ (said actuators might be pneumatic solenoid valves). Thus, if an actuator Aₙ as well as an actuator Aₘ is actuated, only at position (n,m) both microfabricated valves v1 and v2 from the RFCP geometry are closed/actuated resulting in a removal of a matrix immobilized at this position as described previously. Multiple microfabricated compartments having a RFCP geometry might be perfused with the same fluid by connecting said microfabricated compartments at node N₂₄ in a way that the same hydrodynamic pressure p₁ is applied to all microfabricated chambers. In addition, all nodes N₁₃ from said microfabricated chambers might be connected so that all microfabricated compartment have the same hydrodynamic pressure p₂ at node N₁₃. Thus, matrices that are removed using said RFCP geometry might move to a common microfabricated channel which might be defined as collection channel. Said collection channel might be connected to a common outlet that enables the transfer of removed matrix into another format. This has the advantage that any position (n,m) within said array having n×m positions can be addressed by using only n+m actuators instead of n×m actuators.

Removing multiple matrices simultaneously. In another advantageous embodiment, multiple positions within said n×m array might be addressed simultaneously. For example, a first actuator Aₙ₁, a second actuator Aₙ₂ and a third actuator Aₘ₁ might be actuated simultaneously. This leads to a simultaneous removal of matrices located at the positions (n₁, m₁) and (n₂, m₁). The simultaneous removal of immobilized matrices has the advantage that the time needed for removing said matrices is dramatically removed.

Immobilization and removal of two matrices. In another advantageous embodiment, two matrices of the same or of different type that are located at a certain position (n,m) within a microfabricated compartment which is part of a RFCP geometry might be sequentially removed (FIGS. 13 and 14). To this end, two matrices are positioned in close proximity or in contact within a microfabricated compartment. Said microfabricated compartment might have a bypass channel with the hydrodynamic resistance R_{bypass}=2×R₅ as well as a microfabricated geometry for the immobilization of two matrices having the resistance R_{Trapping Geometry}=R₃+(R₄⁻¹+R₄⁻¹+(R₁+R₂)⁻¹)⁻¹ (FIG. 13). During the immobilization of matrices, the main volume flow might flow from node N3 to NO through the hydrodynamic resistance R_{Trapping Geometry} as R_{Trapping Geometry} might be smaller than the resistance of the bypass channel R_{bypass}. If a first enters the trapping geometry, the hydrodynamic resistance R_{Trapping Geometry} increases but remains smaller than the resistance of the bypass channel. Thus, a second matrix entering said microfabricated trapping geometry enters the trapping geometry and the hydrodynamic resistance of said trapping geometry increases so that R_{Trapping Geometry}>>R_{bypass}. A third matrix might enter the bypass channel and move to the next microfabricated compartment. Due to the described hydrodynamic resistances, applying a reverse flow results in a force acting on the trapped matrices with a force F₁ acting on matrix 1 (31A) positioned at node N, and with a force F₂ acting on matrix 2 (31C) positioned at node N₂ with F₁<F₂. A critical force F_{crit,n} might be needed to remove a matrix n located at position n within a microfabricated compartment. For example, F_{crit,1} is the force necessary to remove a matrix located at position 1 and F_{crit,2} is the force necessary to remove a matrix located at position 2. The forces acting on said matrices dependent on the applied pressure difference between the nodes N₃ and N₄. If all matrices have to experience the same force F_{crit} to be removed from the microfabricated trapping geometry the reverse flow rate for removing matrix 2 may be increased until F₂ equals F_{crit}. The force acting on the matrices 1 and 2 is F₁ and F₂ respectively with F₁<F₂ and F₁<F_{crit}. Thus, only the matrix 2 is removed while matrix 1 stays within its position. A further increase of the flow rate and thus the pressure difference might result in a force F₁ acting on matrix 1 that equals F_{crit} which leads to a removal of matrix 1.

This has the main advantage that immobilized matrices can be removed sequentially. For example, a matrix located at position 2 might be removed and collected within a first well of a 96-well plate or another format. Afterwards, a matrix located at position 1 might be removed and collected within a second well. Another advantage is that one matrix might be paired with various second matrices in a sequential manner. For example, matrix of type 1 might first be positioned next to matrix of type 2. Matrix of type 2 might be removed after a certain period and a new matrix might be positioned next to matrix of type 1. This process might be repeated several times.

Immobilization and removal of three matrices. In another advantageous embodiment, three matrices of the same or of different type that are located at a certain position (n,m) within a microfabricated compartment which is part of a RFCP geometry might be sequentially removed. To this end, matrices might be first immobilized as described previously (FIG. 15, FIG. 16 and FIG. 17). Applying a reverse flow results in a force acting on the trapped matrices with a force F1 acting on matrix 1 (31A) positioned at node N1 and with a force F2 acting on matrix 2 (31B) positioned at node N2 with F1<F2. Applying a reverse flow results in a force acting on the trapped matrices with a force F1 acting on matrix 1 positioned at node N1, with a force F2 acting on matrix 2 positioned at node N2 and with a force F3 acting on matrix 3 (31C) positioned at node N3 with F1<F2<F3. Applying a reverse flow results in a force acting on the trapped matrices with a force F1 acting on matrix 1 positioned at node N1, with a force F2 acting on matrix 2 positioned at node N2 and with a force Fn acting on matrix n positioned at node Nn with F1<F2< . . . <Fn. A critical force Fcrit,n is needed to remove a matrix n located at position n within a microfabricated compartment. The forces acting on said matrices dependent on the applied pressure difference. If all matrices have to experience the same force Fcrit to be removed from the microfabricated trapping geometry the reverse flow rate for removing matrix 3 may be increased until F3 equals Fcrit. The force acting on the matrices 1 and 2 is F1 and F2 respectively with F1<F2<F3 and F1<F2<Fcrit. Thus, only the matrix 3 is removed while matrix 1 and matrix 2 stay within their position. A further increase of the flow rate might result in a force F2 acting on matrix 2 that equals Fcrit which leads to a removal of matrix 2 while matrix 1 stays in place. Finally, a further increase of the flow rate might result in a force F1 acting on matrix 1 which is equal to Fcrit. Thus, the matrix 1 is removed. This has the main advantage, that immobilized matrices can be removed sequentially. For example, a matrix located at position 3 might be removed and collected within a first well of a 96-well plate or another format. Afterwards, a matrix located at position 2 might be removed and collected within a second well.

Immobilization and removal of more than three matrices. In another advantageous embodiment, more than three matrices of the same or of different type that are located at a certain position (n,m) within a microfabricated compartment which is part of a RFCP geometry might be sequentially removed. To this end, matrices might be first immobilized as described previously so that multiple matrices might be positioned in a sequence. Said matrices might be located within a microfabricated trapping geometry in which each matrix experiences a different force deepening on its trapping position. Applying a reverse flow results in a force acting on the trapped matrices with a force F₁ acting on matrix 1 positioned at node N₁, with a force F₂ acting on matrix 2 positioned at node N₂ and with a force Fₖ acting on matrix k positioned at node Nₖ with F₁<F2< . . . <Fₖ. A critical force F_{crit,k} might be needed to remove a matrix k located at position k within a microfabricated compartment. The forces acting on said matrices dependent on the applied pressure difference. If all matrices have to experience the same force F_{crit} to be removed from the microfabricated trapping geometry the reverse flow rate for removing matrix k may be increased until Fₖ equals F_{crit}. The force acting on the matrices 1, 2 . . . k is F₁, F₂ . . . Fₖ respectively with F₁<F₂< . . . <Fₙ and F₁<F₂< . . . <F_{crit}. Thus, only the matrix k is removed while all matrices 1, 2 . . . k−1 stay within their position. A further increase of the flow rate might result in a force Fₖ1 acting on matrix k−1 that equals F_{crit} which leads to a removal of matrix k−1 while matrix k−2 stays in place. Finally, this process might be repeated until all matrices have been removed. This has the main advantage, that multiple immobilized matrices can be removed sequentially and transferred into a 96-well plate or another format.

Extraction of cells located within immobilized matrices and subsequent transfer into another format—Highly controlled cell transfer using RFCP. In another advantages embodiment, said array might be used to transfer single or multiple cells located within a matrix that is positioned within said array to another format such as a 96-well plate, a 384-well plate, a 1536-well plate or a microwell plate whereas exactly one single cell might be transferred to a pre-defined well of said established formats or each similar formats. For example, a matrix might contain initially one single cell. After cultivation for a certain time period (e.g. 3 days) said single cell might divide and proliferate and might form a spheroid consisting of more than one. The encapsulated cells might be separated from each other and subsequently transferred into another format whereas each well of said format will only contain one single cell derived from said matrix. For example, said extraction process might be performed in the following steps:
1. Immobilization of cell-laden matrices. Immobilization of matrices containing single or multiple cells within a positioner, in particular trapping structure at which the flow can be reversed using the previously mentioned RFCP mechanism.
2. Optionally: Cell cultivation within matrices. Cultivation of cells for an extended time period (For example cells might be cultivated for one, two or more than three days up to several weeks).
3. Event-triggered removal of immobilized matrices. As soon as a certain event occurs, the matrix containing said cells is removed from the trap by said RFCP mechanism and transferred to a perfusion compartment containing a filter structure that holds the matrix in place and allows smaller matrix to pass through. For example, said event might be a certain fluorescence intensity of the cultivated cells (e.g. cultivated cells might express a fluorescent reporter protein), a certain cell morphology such as an increased cell size, the formation of a cell spheroid with a certain size or a certain surface profile.
4. Extraction of single cells from matrices. The cell-laden matrix that is hold in place at the filter structure is then perfused with a solution that enables the separation of aggregated cells that might be attached due to cell-cell or cell-matrix contacts. Said solution might contain for example a protease (e.g. trypsin) for digesting surface proteins that mediate cell-cell contacts as well as cell-cell and cell-matrix adhesion. Afterwards, the matrix that contains now separated cells is dissolved. In particular, this might be done by perfusion with metalloproteases for that contain degradation sites that can be cleaved by metalloproteases for digestion.
5. Refocusing of single cells. Cells that are released from the matrix due to matrix removal are further separated from each other by using a re-focusing geometry or by using multiple re-focusing geometries in sequence.
6. Trapping within RFCP geometries. Re-focused cells might be trapped in a single cell trap located within a RFCP geometry. Multiple RFCP traps might be positioned in sequence connected with each other.
7. Transfer of trapped single cells into a standard format. Afterwards, single cells located with said RFCP geometries might be transferred to a standard format such as the well by actuating the corresponding valves as described previously.

This has the advantage that cells derived from one single cells can be separated and further analysed with conventional methods such as RT-PCR or single-cell sequencing without losing the time-lapse information about the cultured cells that has been recorded during cell culture. For example, this time-lapse information might be among others growth data, fluorescence data or migration data.

Use of a Cell Culture Plate

According to one embodiment, the cell-laden matrix is provided in a cell culture plate. Details of the cell culture plate (e.g. a 12, 24, 96, 384 etc. well-plate) and the cell-laden matrix are described elsewhere herein. In particular, the matrix may be provided by a hydrogel. The matrix may be three-dimensional and e.g. at least partially ellipsoidal, preferably plug or semi-sphere shaped. In one embodiment, a cell-laden matrix comprising at least one cell is provided per compartment (e.g. well) of the cell culture device. The cell-laden matrix may comprise more than one cell. Examples are a cell colony or one or more different cell types as disclosed herein. In one embodiment, the cell-laden matrix is provided in the compartment such that liquid that may surround the cell-laden matrix can be removed or exchanged without affecting the cell-laden matrix.

In particular, the cell-laden matrix is provided by a three dimensional hydrogel comprising more than one cell, wherein the cell-laden matrix is provided in the compartment such that liquid that may surround the cell-laden matrix can be removed or exchanged without affecting the cell-laden matrix. A single cell-laden matrix may be provided per compartment that comprises a cell-laden matrix. The cell-laden matrix may for instance prepared by transferring a solution comprising the matrix material and the at least one cell into the compartment of the cell culture plate. After transferal, the solution forms the matrix generating the cell-laden matrix.

In one embodiment, the cell-laden matrix is incubated in a compartment of said cell culture plate, e.g. a well of a well plate. The cell-laden matrix in the compartment of the cell culture plate may be surrounded at least partially by a liquid. The liquid can be present during the incubation. The liquid preferably covers the cell-laden matrix completely during incubation in order to avoid drying of the matrix. Liquids suitable for incubation of the cell-laden matrix have been described herein and it is referred thereto. In a particular embodiment, the cell-laden matrix in the compartment is covered by cell culture media.

Incubation of the cell-laden matrix optionally takes place before being in fluidic contact with the capture matrix. The released one or more biomolecules of interest may thus accumulate in the compartment, in particular in the liquid comprised in the compartment.

A suitable incubation period in particular depends on the cell type comprised in the cell-laden matrix and can be determined by the skilled person in relation to the used cells and the biomolecule(s) of interest. A suitable incubation period can be selected in embodiments from the range of 1 h to 72 h, such as 4 h to 72 h. A shorter incubation period (e.g. 1 h to 24 h) may be selected for microbiological applications. For instance, a shorter incubation period may be selected for a prokaryotic cell, such as a bacterial cell, which can be comprised in the cell-laden matrix as disclosed herein. A longer incubation period (e.g. 4 h to 72 h) may be selected for other applications. For instance, a longer incubation period may be selected for a eukaryotic cell, such an animal cell, which can be comprised in the cell-laden matrix.

After incubation, the capture matrix may be added to the compartment for binding the released biomolecule(s) of interest. Other contacting orders are also within the scope of the present disclosure as described herein. Furthermore, the liquid in the compartment comprising the one or more biomolecules of interest can be obtained and added to the capture matrix. In such embodiment, the capture matrix may also be present in a different compartment. In one embodiment, more than one capture matrix is provided and contacted with the one or more cell-laden matrix, respectively the released biomolecule(s) of interest. Capture matrices have been described elsewhere herein and it is referred to the respective disclosure. After binding the one or more biomolecules of interest, the capture matrix is optionally transferred to another compartment, such as a compartment of a cell culture plate. The capture matrix may also be introduced into a microfabricated cell culture device as described herein.

Afterwards, the remaining cell-laden matrix inside the compartment of the cell culture plate can be incubated again and a fresh capture matrix may be added. This advantageously allows to acquire time-lapse secretion profiled of biomolecules of interest, as disclosed herein. Suitable time intervals for measurement have been disclosed herein and it is referred thereto.

Details are also described in PCT/EP2020/056975, herein incorporated by reference.

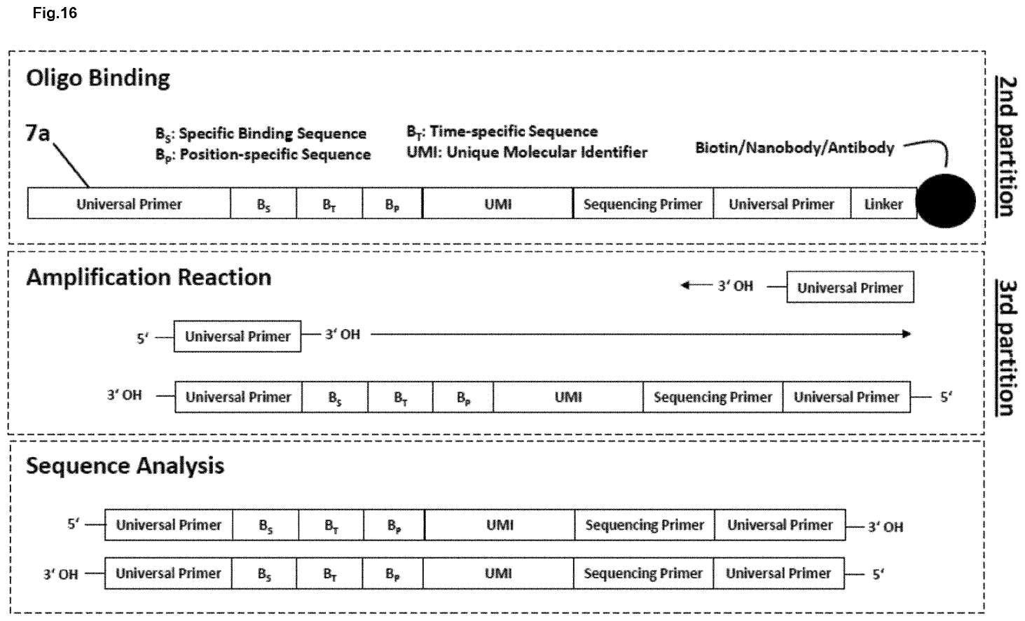

In another advantageous embodiment, the processing of the capture matrix is separated from the cell-laden matrix thereby preventing any (side-)effects on cell(s) located within said cell-laden matrices. To this end, a capture matrix and at least one cell-laden matrix are incubated within a first closed compartment as described before. After a defined incubation time (e.g. 1 h, 2 h or more), the first compartment is selectively opened and the capture matrix is transferred to a second compartment e.g. by using one or more microfabricated valves, preferably by a device comprising a valve arrangement which is adapted to selectively change the direction of fluid passing the location (e.g. RFCP geometry). The second compartment contains a positioning mean (e.g. a hydrodynamic trap) located within the compartment for trapping of the capture matrix and subsequent controlled transfer into another format. Thus, in one exemplary embodiment the exit portion of the first compartment is connected to the feeding channel of the second compartment as illustrated exemplary in FIG. 20. The second compartment can subsequently be perfused with various solutions without influencing the first compartment which still contains single or multiple cell-laden matrices. Thus, the capture matrix containing one or more types of capture molecules and thereto bound biomolecules of interest are first transferred to the second compartment, where the capture matrix is then perfused e.g. with different solutions such as PBS, a blocking solution, a solution containing detection molecules comprising a barcode label, a solution containing one or more oligonucleotides e.g. comprising a barcode for the current time point/UMI sequence, solution for performing a polymerization extension reaction.

The separation of the capture matrix processing and the position of the cell-laden matrices offers several advantages. Firstly, the cell-laden matrix does not come into contact with any solutions or buffers that might influence cell behaviour. Secondly, if cell(s) located within said matrix continue to release biomolecules after said incubation time, said biomolecules cannot be quantified during the processing of the capture matrix as the compartment has to be perfused with different solutions thereby washing away any additionally released biomolecules. Thirdly, a new capture matrix that does not have bound any biomolecules of interest can be positioned next to the cell-laden matrix, as soon as the capture matrix having bound thereto the biomolecules of interest is removed.

### FURTHER EMBODIMENTS

Further general characteristics and embodiments are disclosed in the following.

According to another embodiment, the method can be conducted utilizing a microfabricated cell culture device and/or a collection position, which is preferably a collection well (e.g. from a well plate). Also, mixtures of both can be utilized, for instance by performing method steps partially on the microfabricated cell culture device and partially at a collection position.

According to one embodiment, the method according to the first aspect can have one or more of the following characteristics:
i) the method measures and optionally, quantifies biomolecules secreted by at least one cell;
ii) capture of molecules is performed during cultivation/incubation of at least one cell; analysis of captured molecules is either performed during cultivation/incubation or afterwards
iii) a time-dependent analysis of secreted biomolecules is performed, wherein the method allows analyzing multiple biomolecules at multiple time points;
iv) biomolecules are analyzed time-dependently, wherein time points are selected from 1 to 100 or more, preferably 2 to 90, 3 to 80, 4 to 70, 5 to 60, or 5 to 50, more preferably 6 to 40, 7, to 30, or 8 to 20;
v) biomolecules are analyzed time-dependently, wherein the time interval between analyses is selected from ≥10 min, ≥20 min, ≥30 min, ≥1 h, ≥2 h, ≥3 h, ≥4 h, 5 h or more, up to days 1 d, 2 d or several days;
vi) the capture matrix and the cell-laden matrix are incubated within the same compartment
vii) after incubating the cell-laden matrix for a pre-defined period, the capture matrix is added to the compartment, wherein the cell-laden matrix is surrounded by a water-immiscible fluid layer;
viii) after capturing the biomolecules of interest, molecules that have not bound to the one or more types of capture molecules are removed which may be done by flushing the compartment with a fluid;
ix) the method is performed in an automated manner.

Coupling of Phenotypic and Genotypic Information

Another advantage of said disclosure is that cells can be cultivated over an extended period at n×m positions. During the cultivation period, released molecules can be captured and processed and subsequently analysed and quantified. In addition, cells can be removed from positions n×m at any time point and as soon as a defined requirement is fulfilled.

Afterwards, removed cells might be analysed with conventional methods such as qRT-PCR or sequencing. Thus, a further critical advantage is the coupling of various cell specific data including:
Phenotypic data, such as:
   time-lapse microscopy data such as fluorescent data that can be gained for example by using cells expressing a fluorescent reporter molecule or by using fluorescent probes such as live cell membrane stainings. In addition, data such as the cell shape, cell migration and cell viability (e.g. formation of apoptotic bodies), formation of lamellipodia, may be derived from the time-lapse microscopy data to gain more information about the cell phenotype.
   Time-lapse secretion profiles gained as disclosed
   Surface marker profiles
   Intracellular phenotypic data gained by using techniques such as immunostaining
Genotypic data for example gained from techniques such as qRT-PCR or sequencing

For example, a single cell located within a cell-laden matrix at position (n, m) might express a fluorescent protein that is coupled to a specific promotor. The single cell might start to proliferate resulting in a small cell colony. In addition, during the cell cultivation, the cell may release various molecules (e.g. via secretion) that can be analysed using the current disclose. As soon as the fluorescent signal of said colony reaches a certain value the matrix located at position (n, m) containing said colony might be removed and analyzed with qRT-PCR or NGS. Thus, the current disclose provides the unique advantage to combine various time-lapse phenotypic data with the underlying genotype on a single-cell level and is applicable to hundreds to thousands of cell simultaneously.

In another embodiment, previously described methods can be performed within an array of compartments, provided by a microfabricated cell culture device. This enables the simultaneous determination of time-lapse secretion profiles of (single) cell(s) located in hundreds to thousands of compartments.

One compartment for incubation and detection bead processing. To this end, multiple compartments (comprised in the cell culture device, preferably the microfabricated cell culture device) are connected in series sharing a common feeding line that is used for the delivery of capture matrices and cell-laden matrices as disclosed in PCT/EP2018/074527. In addition, each compartment can be perfused individually without affecting other compartments by using a perfusion line. An exemplary embodiment is described in the present disclosure. It is furthermore referred to the following Figure of PCT/EP2018/074526, including the corresponding figure description, which both are herein incorporated by reference:
FIG. 2. The figure illustrates the structure of a generic array that can be used for the handling (e.g. positioning, incubation and removal) of capture matrix and cell-laden matrices (Array of RFCP geometry)

Thus, the feeding line is used for initial loading of the multiple compartments with cell-laden matrices, as well as with capture matrices. Afterwards, the compartments are selectively closed for generating an isolated compartment and thus a defined reaction volume. The processing of the capture matrix can be performed using the perfusion line. Thus, the required different solutions (such as detection molecule solution, washing solution, etc.) can be delivered using the perfusion line or the feeding line.

Removal of a capture matrix located at a defined position can be performed using a valve arrangement which is adapted to selectively change the direction of fluid passing the location (e.g. RFCP mechanism) as disclosed in PCT/EP2018/074527 by addressing the corresponding row and column valves. Capture matrices that do not have bound any biomolecule(s) of interest can be delivered again via the feeding line (e.g. all compartments are perfused with a solution containing capture matrices that do not have bound thereto any biomolecules of interest).

Two Compartments, One for Incubation and One for Capture Matrix Processing.

If the capture matrix processing is spatially separated from the location at which the cell-laden matrices are positioned, two RFCP geometries can be connected and arranged within an array. An illustration of the structure of an array containing separated RFCP geometries, one for processing the capture matrix (processing chamber) and one for cell culture and binding of the biomolecules of interest (e.g. microfluidic cell culture compartment) is given in FIG. 20 of PCT/EP2020/056975, herein incorporated by reference. The separation of the capture matrix processing from the compartment containing the cell-laden matrix/matrices is advantageous as it prevents that the processing of the capture matrix influences cell(s) located within the cell-laden matrices.

The matrices for cell encapsulation and biomolecule if interest capture might have the same or different sizes and might be composed of the same material or a different material.

In one embodiment, both matrices might have a spherical shape with a diameter of 80 μm.

In another aspect of the invention, a method for detecting a plurality of biomolecules of interest, in particular proteins, secreted from a single cell is disclosed.

Also disclosed as part of the present invention are the following embodiments:
1. A method for analyzing one or more cell released biomolecules, comprising providing a cell-laden matrix, wherein the cell-laden matrix comprises at least one cell that releases one or more biomolecules of interest or wherein the cell-laden matrix comprised at least one cell that has released one or more biomolecules of interest, wherein the method comprises the following steps:
a) providing a capture matrix, wherein the capture matrix comprises one or more types of capture molecules, wherein each type of capture molecule binds a biomolecule of interest,
b) incubating the cell-laden matrix to allow release of the one or more biomolecules of interest and binding the one or more biomolecules of interest to the one or more types of capture molecules of the capture matrix thereby providing a loaded capture matrix; c) optionally further processing the loaded capture matrix;
d) using one or more types of detection molecules comprising a barcode label which comprises a barcode sequence (B_{S}) indicative for a target biomolecule of interest for generating an amplifiable molecule that comprises the barcode sequence (B_{S}) and/or reverse complement thereof.
2. The method according to embodiment 1, comprising
e) using the amplifiable molecule for generating a sequenceable product, optionally wherein step e) comprises performing a polymerase chain reaction or an isothermal amplification reaction;
f) optionally sequencing the sequenceable product generated in step e) or the amplifiable molecule generated in step d), and
g) optionally evaluating the sequencing data obtained in step f), wherein evaluating preferably comprises analyzing the obtained sequencing data to determine the presence or absence of the one or more target biomolecules of interest.
3. The method according to embodiment 1 or 2, wherein the barcode label of a detection molecule used in step d) comprises at least one nuclease target site (NTS) and an analyte specific sequence (ASS) that is specific for a biomolecule of interest.
4. The method of embodiment 3, wherein step d) comprises hybridizing the barcode label of the detection molecule to an oligonucleotide that is associated with the loaded capture matrix, wherein said associated oligonucleotide comprises
   at least one nuclease target site (NTS′) that is complementary to the at least one nuclease target site (NTS) of the barcode label of the detection molecule, and
   at least one analyte specific sequence (ASS′) that is complementary to the analyte specific sequence (ASS) of the barcode label of the detection molecule, whereby an at least partially double-stranded hybrid molecule is formed that comprises (i) at least one cleavable recognition site for an endonuclease that is provided by the hybridized nuclease target sites and (ii) the hybridized analyte specific sequences.
5. The method of embodiment 4, wherein in the barcode label, the analyte specific sequence (ASS) is arranged 3′ to the at least one nuclease target site (NTS) and wherein in the oligonucleotide, the analyte specific sequence (ASS′) is arranged 5′ to the at least one nuclease target site (NTS′).
6. The method according to any one of embodiments 3 to 5, wherein the barcode label of the detection molecule comprises a blocked 3′OH end which prevents elongation of the barcode label at the 3′ end.
7. The method according to any one of embodiments 3 to 6, wherein the barcode label of the detection molecule comprises
(i) the at least one nuclease target site (NTS);
(ii) an analyte specific sequence (ASS) which is located 3′ to the at least one nuclease target site (NTS);
(iii) the barcode sequence (B_{S}) which is located 5′ to the at least one nuclease target site (NTS);
and wherein the barcode label additionally comprises
(iv) optionally a sequencing primer target sequence, which is located 5′ to the barcode sequence (B_{S});
(v) a primer target sequence, preferably a universal primer target sequence, which is located 5′ to the barcode sequence (B_{S}) and, if present, 5′ to the sequencing primer target sequence;
(vi) optionally a universal adapter sequence, which is located 5′ to the at least one nuclease target site (NTS) and 3′ to the barcode sequence (B_{S}); and
(vii) optionally a unique molecular identifier (UMI) sequence, which is located 5′ to the at least one nuclease target site (NTS) and, if present, 5′ to the universal adapter sequence.
8. The method according to embodiment 7, wherein the barcode label additionally comprises 5′ to the at least one nuclease target site (NTS) and, if present, 5′ to the universal adapter sequence a barcode sequence (B_{T}) for indicating a time information and/or a barcode sequence (B_{P}) for indicating a position information.
9. The method according to any one of embodiments 4 to 8, wherein step d) comprises cleaving the at least partially double-stranded hybrid molecule at the at least one cleavable recognition site using an endonuclease that recognizes said recognition site thereby providing a cleaved barcode label comprising a free 3′OH end at the cleaved nuclease target site (NTS*).
10. The method according to embodiment 9, wherein step d) comprises extending the cleaved and optionally further processed barcode label in an extension reaction at its 3′ end to provide an amplifiable molecule that comprises at least one sequence from the extension reaction.
11. The method according to embodiment 10, wherein said amplifiable molecule is provided in the extension reaction by hybridizing an extension oligonucleotide to the cleaved and optionally further processed barcode label, wherein the extension oligonucleotide provides a template for the extension reaction, wherein preferably, an amplifiable molecule is generated in the extension reaction which includes a universal primer target sequence added at the 3′ end of the barcode label, wherein such amplifiable molecule is provided by hybridizing an extension oligonucleotide to the cleaved and optionally further processed barcode label, wherein the extension oligonucleotide provides the template for adding in the extension reaction the universal target primer sequence at the 3′ end of the barcode label.
12. The method according to embodiment 10 or 11, in particular when dependent on embodiment 7, characterized in that
(a) the extension oligonucleotide has one or more of the following characteristics:
(i) it comprises a blocked 3′OH end which prevents elongation of the extension oligonucleotide,
(ii) it comprises one or more sequences complementary to one or more sequences of the cleaved barcode label,
(iii) it comprises a universal primer target sequence,
(iv) it comprises one or more nucleotides that is/are complementary to the nucleotides of the cleaved nuclease target site (NTS*) of the cleaved barcode label;
(b) the barcode label comprises the universal adapter sequence and wherein the extension oligonucleotide comprises a sequence that is complementary to the universal adapter sequence;
(c) the barcode label comprises the universal adapter sequence and wherein the extension oligonucleotide hybridizes to the cleaved nuclease target site (NTS*) and the universal adapter sequence of the cleaved barcode label;
(d) the method comprises releasing the associated oligonucleotide from the loaded capture matrix prior to performing the hybridization reaction with the barcode label;
(e) the method comprises separating cleaved barcode labels from intact and thus uncleaved barcode labels; and/or
(f) the method comprises obtaining the cleaved barcode labels and transferring the cleaved barcode labels to a compartment of a device comprising a plurality compartments, such as a 96-well plate, a 384-well plate, a 1536-well plate.
13. The method according to any one of embodiments 10 to 12 when dependent on embodiment 7, wherein the barcode label comprises the universal adapter sequence and wherein the extension reaction provides an amplifiable molecule which comprises from 5′ to 3′ a universal primer target sequence, optionally a sequencing primer target sequence, the barcode sequence (B_{S}), the universal adapter sequence, optionally the cleaved nuclease target site (NTS*), and the universal primer target sequence added in the extension reaction, optionally wherein the amplifiable molecule additionally comprises
a unique molecular identifier (UMI) sequence,
a barcode sequence (B_{T}), and
a barcode sequence (B_{P}),
located 5′ to the universal adapter sequence and 3′ to the universal primer target sequence and, if present, 3′ to the sequencing primer target sequence.
14. The method according to embodiment 13, comprising
e) generating a sequenceable product by amplifying the amplifiable molecule using a primer or primer combination,
optionally wherein a primer combination is used, wherein said primer combination comprises a first universal primer that hybridizes to the added universal primer sequence located 3′ to the cleaved nuclease target site (NTS*) and wherein a second universal primer hybridizes to the reverse complement of the universal primer sequence located at the 5′ end of the amplifiable molecule.
15. The method according to any one of embodiments 7 to 10 when depending on embodiment 7, wherein the barcode label of the detection molecule comprises a universal primer target sequence which is located 5′ to the barcode sequence (B_{S}) and, if present, 5′ to the sequencing primer target sequence and wherein said barcode label comprises a free 3′OH end and a further universal primer target sequence 5′ to the at least one nuclease target site (NTS) and 3′ to the barcode sequence (B_{S}).
16. The method according to embodiment 15 when dependent on embodiment 4, wherein step d) comprises
   hybridizing the barcode label of the detection molecule to the oligonucleotide that is associated with the loaded capture matrix whereby an at least partially double-stranded hybrid molecule is formed that comprises in a double-stranded portion of said molecule (i) at least one cleavable recognition site for an endonuclease and (ii) the hybridized analyte specific sequences, and
   cleaving the at least partially double-stranded hybrid molecule at the at least one cleavable recognition site using an endonuclease that recognizes said recognition site thereby releasing an amplifiable product comprising a free 3′OH end and the remaining sequences of the barcode label including two universal primer target sites that flank at least the comprised barcode sequence(s), wherein the method comprises separating the cleaved barcode labels of the detection molecules providing the amplifiable product from uncleaved detection molecules, e.g. by obtaining the cleaved barcode labels and transferring the cleaved barcode labels to a compartment of a device comprising a plurality compartments, such as a 96-well plate, a 384-well plate, a 1536-well plate.
17. The method according to embodiment 16, wherein the method comprises
e) generating a sequenceable product by amplifying the amplifiable molecule using a universal primer or primer combination capable of hybridizing to at least one of the universal primer target sites of the amplifiable product,
optionally wherein a first universal primer hybridizes to the universal primer sequence that was in the barcode label located 5′ to the nuclease target site (NTS) and wherein a second universal primer hybridizes to the reverse complement of the universal primer sequence located at the 5′ end of the amplifiable molecule, wherein the first and second universal primers are the same of different.
18. The method according to any one of embodiments 3 to 17, wherein the barcode label comprises two or more nuclease target sites (NTS), optionally wherein the two or more nuclease target sites (NTS) provide when present in a double-stranded hybrid recognition sites for two or more different endonucleases.
19. The method according to embodiment 18, wherein the two or more nuclease target sites (NTS) are collocated in the barcode label, optionally separated by one or more further sequences, wherein if present, the associated oligonucleotide comprises sequences complementary thereto to facilitate hybridization.
20. The method according to embodiment 19, wherein the analyte specific sequences (ASS) is located in-between two nuclease target sites (NTS).
21. The method according to any one of embodiments 18 to 20 when dependent on embodiment 4, characterized by one or more of the following features:
(aa) two or more cleavable recognition sites for different endonucleases are present in the at least partially double-stranded hybrid and step d) comprises cleaving the at least partially double-stranded hybrid molecule using two or more different endonucleases that recognizes said recognition sites;
(bb) the endonuclease is a restriction endonuclease;
(cc) the endonuclease is a restriction endonuclease that recognizes a specific nucleotide sequence as cleavable recognition site in the double-stranded DNA hybrid that is formed between the barcode label and the oligonucleotide, optionally wherein the restriction endonuclease cuts the cleavable recognition site so that no more than 5 nucleotides, no more than 4 nucleotides, no more than 3 nucleotides or not more than 2 nucleotides of the original nuclease target site remain at the 3′ end of the cleaved nuclease target site (NTS*) of the barcode label;
(dd) the endonuclease is a type II restriction endonuclease;
(ee) the endonuclease is a commercially available restriction endonuclease, optionally selected from BamHI, BgIII, ClaI, EcoRI, HindIII, PstI, SalI and XmaI.
22. The method according to any one of embodiments 2 to 21, comprising
f) sequencing the sequenceable product generated in step e).
23. The method according to any one of embodiments 4 to 22, wherein the barcode label of the detection molecule hybridizes to the oligonucleotide, wherein hybridization involves the analyte specific sequence (ASS), wherein
   the barcode labels of one type of detection molecule are the same, thereby hybridizing to oligonucleotides of the matching type of oligonucleotide, and
   wherein the barcode labels of another type of detection molecule is different at least in the analyte specific sequence (ASS), thereby hybridizing to the oligonucleotides of another type of oligonucleotides comprising matching sequences complementary to the analyte specific sequence (ASS) of the matching type of detection molecule.
24. The method according to one of embodiments 4 to 23, comprising using two or more types of detection molecules, wherein each type of detection molecule binds to one type of oligonucleotide, in particular by hybridizing via the analyte specific sequence (ASS) of the barcode label to the complementary sequence (ASS′) of the matching type of oligonucleotide, wherein each type of detection molecule comprises a different analyte specific sequence (ASS) and each matching type of oligonucleotide comprises a hybridizing complementary sequence (ASS′).
25. The method according to any one of embodiments 4 to 24, wherein the barcode label comprised in one type of detection molecule is the same and wherein the barcode label comprised in another type of detection molecule differs in
   the barcode sequence (B_{S}) for indicating the specificity, and
   analyte specific sequence (ASS).
26. The method according to one or more of embodiments 4 to 25, wherein different types of detection molecules used at the same time comprise barcode labels that share
(ii) a common a barcode sequence (B_{T}) for indicating a time information, and
(iii) a common barcode sequence (B_{P}) for indicating a position information.
27. The method according to embodiment 26, wherein at least all barcode labels of the same type of detection molecule comprise a unique molecular identifier (UMI) sequence.
28. The method according to any one of embodiments 4 to 27 when dependent on embodiment 4, wherein the oligonucleotide is attached to a secondary capture molecule that binds a biomolecule of interest that is bound by a capture molecule of the capture matrix and wherein the oligonucleotide is associated with the loaded capture matrix by means of said secondary capture molecule.
29. The method of embodiment 28, characterized by one or more of the following features:
(aa) the oligonucleotide is attached via a linker to the secondary capture molecule;
(bb) the oligonucleotide comprises a blocked 3′OH end which prevents elongation of the oligonucleotide;
(cc) the secondary capture molecule is selected from an antibody or antibody binding fragment.
30. The method of embodiment 28 or embodiment 29, wherein the secondary capture molecule comprising the oligonucleotide is added in step c) in order to allow binding of the secondary capture molecule to its biomolecule of interest that is bound by the loaded capture matrix whereby the oligonucleotide becomes associated with the loaded capture matrix.
31. The method according to embodiment 30, wherein step c) comprises washing the loaded capture matrix with the bound secondary capture molecule comprising the oligonucleotide and optionally transferring the loaded capture matrix with the associated oligonucleotide to a new partition.
32. The method according to any one of embodiments 28 to 31, wherein the detection molecule comprises or consists of a single-stranded oligonucleotide.
33. The method according to any one of embodiments 2 to 32, when dependent on embodiment 4, wherein step e) comprises performing an amplification reaction using a primer or primer combination, preferably wherein the primer or primer combination hybridizes to the cleaved or cleaved and extended barcode label and/or a complement thereof.
34. The method according to any one of embodiments 2 to 33, when dependent on embodiment 4, wherein generating a sequenceable reaction product in step f) comprises amplifying the cleaved or cleaved and extended barcode label by performing a polymerase chain reaction or an isothermal amplification reaction.
35. The method according to any one of embodiments 2 to 34, when dependent on embodiment 4, wherein generating a sequenceable reaction product comprises providing one or more primers, preferably a primer pair, optionally wherein the one or more primers hybridize to the cleaved or cleaved and extended barcode label or a complement thereof.
36. The method according to embodiment 35, wherein the one or more primers comprise one or more barcode sequences, wherein the barcode sequences are present on the sequenceable reaction product after generating the sequenceable product.
37. The method according to embodiment 36, wherein the one or more primers comprise a barcode sequence (B_{T}) for indicating a time information; and/or the one or more primers comprise a barcode sequence (B_{P}) for indicating a position information.
38. The method according to any one of embodiments 4 to 37 when dependent on embodiment 4, wherein the 5′ end of the oligonucleotide is attached to the capture matrix and the 3′ end of the oligonucleotide is conjugated to a capture molecule of the capture matrix whereby the oligonucleotide is associated with the capture matrix.
39. The method according to embodiment 38, wherein the detection molecule comprises the barcode label attached to a secondary capture molecule that binds a biomolecule of interest that is bound by a capture molecule of the capture matrix, whereby the barcode label and the oligonucleotide can hybridize to form the at least partially double-stranded hybrid molecule that comprises at least one cleavable recognition site for an endonuclease.
40. The method of embodiment 38 or 39, characterized by one or more of the following features:
(aa) the oligonucleotide is attached via a linker to the capture matrix and/or the capture molecule, wherein the linker preferably is a cleavable linker, and wherein the linker is optionally selected from
(i) hydrazine, hydrazide, disulfides N-succinimidyl-4-(2-pyridyldithio)pentanoate (SPP), N-succinimidyl-4-(2-pyridyldithio)butyrate (SPDB), 4-(4′-acetylphenoxy)butanoic acid (AcBut), dipeptides valine-citrulline (Val-Cit) and phenylalanine-lysine (Phe-Lys), or
(ii) wherein the linker is provided by a sequence that comprises a restriction site that is different from the nuclease target site (NTS) and therefore is not recognized by the endonuclease that is used to cleave the cleavable recognition site formed in the double-stranded hybrid;
(bb) the oligonucleotide is conjugated via an interaction pair to the capture matrix and/or the capture molecule, wherein preferably the interaction pair comprises biotin and a biotin binding polypeptide, wherein the biotin binding polypeptide is preferably selected from streptavidin, tamavidin and avidin, optionally wherein biotin is attached to the oligonucleotide and the biotin binding polypeptide is attached to the capture matrix, which preferably is provided by a synthetic polymer;
(cc) the barcode label is attached via a linker to the secondary capture molecule;
(dd) the secondary capture molecule is selected from an antibody or antibody binding fragment that binds to the biomolecule of interest at a different epitope region than the capture molecule of the capture matrix so that both the capture molecule of the capture matrix and the secondary capture molecule can bind the biomolecule of interest at the same time; and/or
(ee) the barcode label of the detection molecule comprises a blocked 3′OH end which prevents elongation of the barcode label.
41. The method of any one of embodiments 38 to 40, wherein step c) is performed and comprises optionally washing the loaded capture matrix and transferring the loaded capture matrix to a new partition prior to adding the detection molecule comprising the secondary capture molecule and the barcode label.
42. The method according to any one of embodiments 38 to 41, wherein step d) comprises
   adding the secondary capture molecule comprising the barcode label to the loaded capture matrix and binding of the secondary capture molecule to its biomolecule of interest that is bound by the loaded capture matrix, whereby the barcode label and the oligonucleotide are provided in proximity,
   hybridizing the barcode label to the oligonucleotide that is attached to the capture matrix and the capture molecule of the capture matrix to form the at least partially double-stranded hybrid molecule that comprises at least one cleavable recognition site for an endonuclease, and
   cleaving the at least partially double-stranded hybrid molecule at the at least one cleavable recognition site using an endonuclease that recognizes said recognition site thereby releasing an amplifiable product comprising a free 3′OH end.
43. The method according to any one of embodiments 38 to 42, having one or more of the following features:
(a) each type of capture molecule comprises a proximity oligonucleotide, which is different from another type of capture molecule, wherein the proximity oligonucleotides differ in the analyte specific sequence (ASS′) that is complementary to the analyte specific sequence (ASS) of the barcode label of the matching type of detection molecule;
(b) the method comprises obtaining and transferring the loaded capture matrix to a device comprising a plurality of compartments for hybridization of the detection molecule to the proximity oligonucleotide;
(c) the method comprises releasing the one or more types of proximity oligonucleotides from the capture matrix, in particular by degrading or cleaving a linker moiety that attaches the proximity oligonucleotide to the capture matrix, optionally wherein after releasing the proximity oligonucleotide from the capture matrix, the barcode label of the detection molecule hybridizes to the released proximity oligonucleotide;
(d) the method comprises performing an extension reaction, thereby generating an extended barcode label, optionally wherein the extended barcode label comprises
(i) a barcode sequence (B_{S}) for indicating the specificity,
(ii) a barcode sequence (B_{T}) for indicating a time information,
(iii) a barcode sequence (B_{P}) for indicating a position information, and
(iv) one or more primer target sequences, preferably two primer target sequences; and (iv) optionally a unique molecular identifier (UMI) sequence.
44. The method according to any one of embodiments 1 to 43, comprising analyzing Y different target biomolecules of interest using
   Y different types of capture molecules that target Y different target biomolecules of interest and
   Y different types of corresponding detection molecules representative for said Y target biomolecules of interest,
wherein Y is at least 2 and
wherein the barcode label of each type of detection molecule differs in its barcode sequence B_{S} from the barcode sequence B_{S} of all other types of detection molecules to allow identifying the captured target biomolecule of interest based on the barcode sequence B_{S} that is specific for the target biomolecule of interest.
45. The method according to embodiment 44 when depending on embodiment 3, wherein the barcode label of each type of detection molecule comprises an analyte specific sequence (ASS) that is specific for the target biomolecule of interest and differs from the analyte specific sequence (ASS) of all other types of detection molecules.
46. The method according to embodiment 44 or 45 when depending on embodiment 4, wherein for the analysis of at least two different target biomolecule of interests, referred to as analyte (A) and analyte (B), the following matching components are used:
for analyte (A)
(i) a capture molecule (A) that specifically binds the analyte (A) to capture said analyte (A) to the capture matrix;
(ii) a detection molecule (A) for detecting the analyte (A), said detection molecule comprising a barcode label (A) which comprises a barcode sequence B_{S} indicative for the analyte (A), an analyte specific sequence (ASS) that is specific for the analyte (A) and at least one nuclease target site (NTS), optionally wherein the 3′OH end of the barcode label is blocked;
(iii) an oligonucleotide (A) attached to a secondary capture molecule (A) that specifically binds to analyte (A) when it is bound by the capture molecule (A) of the capture matrix, wherein the oligonucleotide (A) comprises
(aa) at least one nuclease target site (NTS′) that is complementary to the at least one nuclease target site (NTS) of the barcode label of the detection molecule (A), and
(bb) at least one analyte specific sequence (ASS′) that is complementary to the analyte specific sequence (ASS) of the barcode label (A) of the detection molecule (A); and
for analyte (B)
(i) a capture molecule (B) that specifically binds the analyte (B) to capture said analyte (B) to the capture matrix;
(ii) a detection molecule (B) for detecting the analyte (B), said detection molecule comprising a barcode label (B) which comprises a barcode sequence B_{S} indicative for the analyte (B), an analyte specific sequence (ASS) that is specific for the analyte (B) and at least one nuclease target site (NTS), optionally wherein the 3′OH end of the barcode label is blocked;
(iii) an oligonucleotide (B) attached to a secondary capture molecule (B) that specifically binds to analyte (B) when it is bound by the capture molecule (B) of the capture matrix, wherein the oligonucleotide (B) comprises
(aa) at least one nuclease target site (NTS′) that is complementary to the at least one nuclease target site (NTS) of the barcode label of the detection molecule (B), and
(bb) at least one analyte specific sequence (ASS′) that is complementary to the analyte specific sequence (ASS) of the barcode label (B) of the detection molecule (B).
47. The method according to embodiment 44 or 45 when depending on embodiment 4, wherein for the analysis of at least two different target biomolecule of interests, referred to as analyte (A) and analyte (B), the following matching components are used:
for analyte (A)
(i) a capture molecule (A) that specifically binds the analyte (A) to capture said analyte (A) to the capture matrix;
(ii) an oligonucleotide (A), wherein the 5′ end of the oligonucleotide is attached to the capture matrix and the 3′ end is conjugated to the capture molecule (A) of the capture matrix, wherein the oligonucleotide (A) comprises
(aa) at least one nuclease target site (NTS′) that is complementary to the at least one nuclease target site (NTS) of the barcode label of the detection molecule (A), and
(bb) at least one analyte specific sequence (ASS′) that is complementary to the analyte specific sequence (ASS) of the barcode label (A) of the detection molecule (A); and
(iii) a detection molecule (A) for detecting the analyte (A), said detection molecule comprising
(aa) a barcode label (A) which comprises a barcode sequence B_{S} indicative for the analyte (A), an analyte specific sequence (ASS) that is specific for the analyte (A) and at least one nuclease target site (NTS), optionally wherein the 3′OH end of the barcode label is blocked, and
(bb) a secondary capture molecule (A) that binds the analyte (A) that is bound by the capture molecule (A) of the capture matrix,
wherein upon binding of the secondary capture molecule (A) to the analyte (A) the barcode label (A) and the oligonucleotide (A) hybridize to form the at least partially double-stranded hybrid molecule that comprises at least one cleavable recognition site for an endonuclease formed by the hybridized nuclease target sites (NTS) and (NTS′);
and for analyte (B)
(i) a capture molecule (B) that specifically binds the analyte (B) to capture said analyte (B) to the capture matrix;
(ii) an oligonucleotide (B), wherein the 5′ end of the oligonucleotide is attached to the capture matrix and the 3′ end is conjugated to the capture molecule (B) of the capture matrix, wherein the oligonucleotide (B) comprises
(aa) at least one nuclease target site (NTS′) that is complementary to the at least one nuclease target site (NTS) of the barcode label of the detection molecule (B), and
(bb) at least one analyte specific sequence (ASS′) that is complementary to the analyte specific sequence (ASS) of the barcode label (B) of the detection molecule (B); and
(iii) a detection molecule (B) for detecting the analyte (B), said detection molecule comprising
(aa) a barcode label (B) which comprises a barcode sequence B_{S} indicative for the analyte (B), an analyte specific sequence (ASS) that is specific for the analyte (B) and at least one nuclease target site (NTS), optionally wherein the 3′OH end of the barcode label is blocked, and
(bb) a secondary capture molecule (B) that binds the analyte (B) that is bound by the capture molecule (B) of the capture matrix,
wherein upon binding of the secondary capture molecule (B) to the analyte (B) the barcode label (B) and the oligonucleotide (B) hybridize to form the at least partially double-stranded hybrid molecule that comprises at least one cleavable recognition site for an endonuclease formed by the hybridized nuclease target sites (NTS) and (NTS′).
48. The method according to embodiment 46 or 47, wherein the method comprises analyzing additional target biomolecules of interest using corresponding matching components for each target biomolecule of interest.
49. The method according to any one of embodiments 44 to 48, wherein two or more different target biomolecules of interest are analyzed in parallel and wherein the different target biomolecules of interest released are identified based on the different barcode sequences B_{S} that are specific for a biomolecule of interest, preferably by sequencing.
50. The method according to any one of embodiments 44 to 49, wherein the method comprises analyzing at least two, at least three, at least 4, or at least 5 different target biomolecules of interest, optionally wherein at least 10 or at least 15 different target biomolecules of interest are analyzed.
51. The method for analyzing one or more cell released biomolecules according to embodiment 1 or 2, comprising providing a cell-laden matrix, wherein the cell-laden matrix comprises at least one cell that releases one or more biomolecules of interest or wherein the cell-laden matrix comprised at least one cell that has released one or more biomolecules of interest, wherein the method comprises the following steps:
a) providing a capture matrix, wherein the capture matrix comprises one or more types of capture molecules, wherein each type of capture molecule binds a biomolecule of interest and is attached to the capture matrix via an proximity oligonucleotide, wherein the 5′ end of the oligonucleotide is attached to the capture matrix and the 3′ end of the oligonucleotide is conjugated to the capture molecule;
b) incubating the cell-laden matrix to allow release of the one or more biomolecules of interest and binding the one or more biomolecules of interest to the one or more types of capture molecules of the capture matrix thereby providing a loaded capture matrix;
c) optionally further processing the loaded capture matrix;
d) using one or more types of detection molecules comprising (i) a barcode label which comprises a barcode sequence (B_{S}) indicative for a specific biomolecule of interest and (ii) a secondary capture molecule that binds to a biomolecule of interest on the loaded capture matrix and binding the biomolecule of interest via the secondary capture molecule and hybridizing at least a portion of the barcode label to the proximity oligonucleotide of the capture matrix and extending the barcode label using the proximity oligonucleotide as template, thereby generating an amplifiable molecule that comprises the barcode sequence B_{S} and sequences resulting from the extension.
52. The method according to embodiment 51, wherein each type of capture molecule is attached to the capture matrix via a different proximity oligonucleotide and wherein in step d) at least the portion of the barcode label that comprises the barcode sequence (B_{S}) is hybridized to the proximity oligonucleotide that is during hybridization optionally still attached to the capture matrix, wherein said proximity oligonucleotide comprises a barcode sequence (B_{S}′) that is complementary to the barcode sequence (B_{S}) of the barcode label and involved in hybrid formation.
53. The method according to embodiment 51 and 52, wherein the proximity oligonucleotide comprises a universal primer sequence and wherein due to the extension of the barcode label using the proximity oligonucleotide as template, the generated amplifiable molecule comprises a corresponding universal primer sequence.
54. The method according to embodiment 53, wherein different types of proximity oligonucleotides that are conjugated to different types of capture molecules comprise the same universal primer sequence.
55. The method of any one of embodiments 51 to 54, characterized by one or more of the following features:
(aa) the proximity oligonucleotide is attached via a linker to the capture matrix and/or the capture molecule;
(bb) the barcode label is attached via a linker to the secondary capture molecule;
(cc) the barcode label comprises a free 3′OH end and can be extended by a polymerase; and/or
(dd) the secondary capture molecule is selected from an antibody or antibody binding fragment that binds to the biomolecule of interest at a different epitope region than the capture molecule of the capture matrix so that both the capture molecule of the capture matrix and the secondary capture molecule can bind the biomolecule of interest at the same time.
56. The method of any one of embodiments 51 to 55, wherein the barcode label comprising the barcode sequence B_{S} additionally comprises
(i) optionally a sequencing primer sequence, which is located 5′ to the barcode sequence (B_{S});
(ii) a universal primer sequence which is located 5′ to the barcode sequence (B_{S}) and, if present, 5′ to the sequencing primer sequence;
(iii) optionally a unique molecular identifier (UMI) sequence, which is located 5′ to the barcode sequence (B_{S}) and 3′ to the universal primer sequence and, if present, 3′ to the sequencing primer sequence.
57. The method according to embodiment 56, wherein the barcode label additionally comprises 5′ to the barcode sequence (B_{S}) and 3′ to the universal primer sequence and, if present, 3′ to the sequencing primer sequence a barcode sequence (B_{T}) for indicating a time information and/or a barcode sequence (B_{P}) for indicating a position information.
58. The method according to any one of embodiments 51 to 57, wherein step c) is performed and comprises optionally washing the loaded capture matrix and transferring the loaded capture matrix to a new partition prior to adding the detection molecule comprising the secondary capture molecule and the barcode label.
59. The method according to any one of embodiments 51 to 58, comprising
e) using the amplifiable molecule for generating a sequenceable product, wherein generating the sequenceable product comprises amplifying the amplifiable molecule.
60. The method according to embodiment 59, when depending on embodiment 53 and 56, wherein step e) comprises using universal primers for the amplification, optionally wherein a first universal primer hybridizes to the universal primer sequence that was incorporated into the amplifiable molecule using the oligonucleotide as template and wherein a second universal primer hybridizes to the reverse complement of the universal primer sequence located 5′ to the barcode sequence (B_{S}) and, if present, 5′ to the sequencing primer sequence.
61. The method according to embodiment 59 or 60, wherein the sequenceable product comprises a barcode sequence (B_{T}) for indicating a time information and/or a barcode sequence (B_{P}) for indicating a position information.
62. The method according to embodiment 61, wherein the barcode sequence (B_{T}) for indicating a time information and/or the barcode sequence (B_{P}) for indicating a position information is introduced during step e) or preferably, via the barcode label of the detection molecule.
63. The method according to any one of embodiments 51 to 62, comprising
f) sequencing the sequenceable product generated in step e).
64. The method according to embodiment 63, comprising using the obtained sequencing information to determine based on the identified barcode sequence (B_{S}) the released one or more biomolecules of interest.
65. The method according to any one of embodiments 51 to 64, comprising providing a capture matrix, wherein the capture matrix comprises one or more types of capture molecules, wherein each type of capture molecule binds a different biomolecule of interest, and wherein each type of capture molecule is attached to a matching type of proximity oligonucleotide, which is different from the matching proximity oligonucleotide of another type of capture molecule.
66. The method according to any one of embodiments 51 to 65, wherein the proximity oligonucleotide is attached to the capture matrix via an interaction pair, preferably an affinity interaction pair, wherein the affinity interaction pair is optionally provided by biotin and a biotin binding polypeptide, wherein the biotin binding polypeptide is preferably selected from tamavidin, streptavidin and avidin.
67. The method according to any one of embodiments 51 to 65, wherein each type of capture molecule comprises a proximity oligonucleotide, which is different from the proximity oligonucleotide of another type of capture molecule, wherein the proximity oligonucleotides at least differ in the barcode sequence (B_{S}′) that is complementary to the barcode sequence (B_{S}) of the detection molecule.
68. The method according to any one of embodiments 51 to 66, wherein the proximity oligonucleotide comprises:
(i) a barcode sequence (B_{S}′), which is complementary to the barcode sequence (B_{S}) of the barcode label of the detection molecule;
(ii) a primer target sequence; and
(iii) a linker moiety; and
(iv) optionally a blocking moiety, wherein preferably the blocking moiety is at the 3′ end of the proximity probe oligonucleotide.
69. The method according to any one of embodiments 51 to 68, wherein two or more different types of capture molecules are attached to the capture matrix using different types of proximity oligonucleotides, wherein each type of capture molecule is attached to the capture matrix using a different type of proximity oligonucleotide, wherein the different types of proximity oligonucleotides differ in the barcode sequence (B_{S}′), which is complementary to the barcode sequence (B_{S}) of the barcode label of the detection label which is indicative for a specific target biomolecule of interest.
70. The method according to any one of embodiments 51 to 69, wherein the method comprises one or more of the following features:
(a) the proximity oligonucleotide is attached to the capture matrix via a linker moiety, optionally wherein the linker moiety has one or more of the following characteristics:
   it is degradable;
   it is degradable on demand, in particular by providing an external stimulus; and/or
   it is degradable by providing one or more of an enzyme, a chemical, a temperature, in particular heat, a pressure, a mechanical force or a light irradiation:
(b) the method comprises releasing the proximity oligonucleotide from the capture matrix, in particular by degrading or cleaving a linker moiety that connects the proximity oligonucleotide with the capture matrix, wherein after releasing, the proximity probe oligonucleotide comprises:
   a barcode sequence (B_{S}′) which is complementary to the barcode sequence (B_{S}) of the barcode label of the detection molecule; and
   a primer target sequence,
optionally wherein after releasing the proximity probe oligonucleotide, the barcode label of the detection molecule hybridizes to the proximity probe oligonucleotide via the complementary sequences (B_{S}) and (B_{S}′).
71. The method for analyzing one or more cell released biomolecules according to embodiment 1 or 2, comprising providing a cell-laden matrix, wherein the cell-laden matrix comprises at least one cell that releases one or more biomolecules of interest or wherein the cell-laden matrix comprised at least one cell that has released one or more biomolecules of interest, wherein the method comprises the following steps:
a) providing a capture matrix, wherein the capture matrix comprises one or more types of capture molecules, wherein each type of capture molecule binds a biomolecule of interest;
b) incubating the cell-laden matrix to allow release of the one or more biomolecules of interest and binding the one or more biomolecules of interest to the one or more types of capture molecules of the capture matrix thereby providing a loaded capture matrix;
c) optionally further processing the loaded capture matrix;
d) using one or more types of detection molecules comprising a barcode label which comprises
(i) a barcode sequence (B_{S}) indicative for a specific biomolecule of interest,
(ii) optionally a sequencing primer sequence, which is located 5′ to the barcode sequence (B_{S}),
(iii) a universal primer sequence which is located 5′ to the barcode sequence (B_{S}) and, if present, 5′ to the sequencing primer sequence,
(iv) optionally a unique molecular identifier (UMI) sequence, which is located 5′ to the barcode sequence (B_{S}) and 3′ to the universal primer sequence and, if present, 3′ to the sequencing primer sequence, and
(v) (iii) a universal primer sequence which is located 3′ to the barcode sequence (B_{S}), and generating an amplifiable molecule that comprises these elements of the barcode label.
72. The method according to embodiment 71, wherein the barcode label additionally comprises flanked by the universal primer sequences a barcode sequence (B_{T}) for indicating a time information and/or a barcode sequence (BE) for indicating a position information.
72. The method according to embodiment 71 or 72, wherein each type of detection molecule specifically binds a biomolecule of interest.
73. The method according to embodiment 72, wherein each type of detection molecule comprises a capture molecule that binds a biomolecule of interest and wherein said capture molecule is conjugated to the barcode label,
optionally wherein the capture molecule is selected from the group consisting of antibodies, intact antibodies having full length heavy and light chains, one or more antibody fragments, such as Fab fragments, Fab′, F(ab′)2, Fv, single domain antibodies, scFv, bi-, tri- or tetra-valent antibodies, Bis-scFv, diabody, triabody, tetrabody and epitope-binding fragments thereof.
74. The method according to any one of embodiments 71 to 72, comprising adding one or more types of tagging molecules, wherein each type of tagging molecule specifically binds a biomolecule of interest.
75. The method according to embodiment 74, wherein the tagging molecule comprises a binding molecule that is capable of binding the biomolecule of interest, optionally wherein the binding molecule of the tagging molecule is selected from the group consisting of antibodies, intact antibodies having full length heavy and light chains, one or more antibody fragments, such as Fab fragments, Fab′, F(ab′)2, Fv, single domain antibodies, scFv, bi-, tri- or tetra-valent antibodies, Bis-scFv, diabody, triabody, tetrabody and epitope-binding fragments thereof.
76. The method according to embodiment 74 or 75, wherein the one or more types of detection molecules used in step d) comprise a binding moiety that is capable of binding the corresponding tagging molecule, whereby a complex is formed that comprises the loaded capture matrix, the tagging molecule bound to the biomolecule of interest and the corresponding detection molecule bound to its tagging molecule, optionally wherein each type of tagging molecule binds its type of detection molecule prior to contacting the tagging molecules with the bound detection molecules with the capture matrix.
77. The method according to any one of embodiments 74 to 76, wherein one or more types of tagging molecules comprise a common interaction moiety that is the same for all types of tagging molecules and wherein the one or more types of detection molecules comprise a common binding moiety that recognizes and binds to the common interaction moiety, optionally wherein the correct assignment of each type of tagging molecule to its type of detection molecule comprising a barcode label comprising a barcode sequence B_{S} that is indicative for the target analyte that is bound by the binding molecule of the tagging molecule is achieved by preparing the combination of each type of tagging molecule bound to its type of detection molecule separate from the combination of other types of tagging molecules bound to its type of detection molecule.
78. The method according to embodiment 76 or 77, wherein the tagging molecule comprises an interaction moiety that is selected from an Fc-region of an antibody, a biotin binding polypeptide and biotin.
79. The method according to any one of embodiments 76 to 78, wherein the interaction moiety of the tagging molecule and the binding moiety of the detection molecule that binds said interaction moiety form part of an interaction pair, wherein said interaction pair is preferably selected from:
(i) biotin and a biotin binding polypeptide, wherein the biotin binding polypeptide is optionally selected from tamavidin, streptavidin and avidin;
(ii) the Fc region of an antibody molecule and an Fc region binding protein, wherein the Fc region binding protein is optionally selected from an anti-Fc antibody, an anti-Fc binding antibody fragment and a nanobody;
(iii) an antigen and an antigen binding polypeptide, wherein the antigen binding polypeptide is preferably selected from an antibody, an antibody binding fragment and a nanobody.
80. The method according to any one of embodiments 74 to 79, wherein more than one type of tagging molecule is provided, wherein each type of tagging molecule has a corresponding type of detection molecule and wherein different types of tagging molecules have different types of detection molecules, wherein the different types of detection molecules at least differ in the barcode sequence B_{S}.
81. The method according to any one of embodiments 74 to 80, wherein more than one type of tagging molecule and more than one type of detection molecule are added simultaneously, wherein each type of resulting molecule combination comprises:
   a binding molecule provided by the tagging molecule for binding a biomolecule of interest, which is different from the binding molecules of the other types of tagging molecules, and
   a barcode label of the detection molecule, wherein the barcode label differs in the barcode sequence B_{S} for each type of tagging molecule.
82. The method according to embodiment 73, having one or more, two or more, or all of the following features:
(i) step c) comprises
   obtaining the capture matrix from a compartment of a cell culture device, which preferably is a microfabricated cell culture device, and
   transferring the capture matrix comprises transferring the capture matrix to a compartment of a processing device, which preferably is a microfabricated processing device;
(ii) the detection molecule comprises the barcode label and a capture molecule for binding a target biomolecule of interest, wherein preferably the capture molecule is an antibody or antibody binding fragment;
(iii) more than one type of detection molecule is added in step d), wherein each type of detection molecule comprises:
   a different capture molecule for binding a different target biomolecule of interest, and
   a different barcode label, wherein the barcode label differs at least in the barcode sequence B_{S} for each type of detection molecule;
83. The method according to any one of embodiments 74 to 81, having one or more, two or more, or all of the following features:
(i) step c) comprises
   obtaining the capture matrix from a compartment of a cell culture device, which preferably is a microfabricated cell culture device, and
   transferring the capture matrix comprises transferring the capture matrix to a compartment of a processing device, which preferably is a microfabricated processing device;
(ii) the one or more types of tagging molecules comprise a binding molecule for binding a target biomolecule of interest, wherein preferably the binding molecule is an antibody or antibody fragment;
(iii) the one or more types of tagging molecules and the one or more types of detection molecules are provided together generating one or more types of associated molecules, in particular wherein associating the detection molecule to the tagging molecule is mediated by an interaction pair, wherein preferably, the interaction pair is an affinity interaction pair, optionally provided by biotin and a biotin binding polypeptide; and/or
(iv) each type of tagging molecule and each type of detection molecule are provided together, thereby generating one type of associated molecule, preferably wherein the one type of associated molecule is generated before contacting said type of associated molecule with the loaded capture matrix for binding the target biomolecule of interest.
84. The method according to embodiment 82 or 83, wherein
(i) the method comprises transferring the capture matrix to another compartment before generating a sequenceable reaction product, wherein the compartment is provided by another device comprising a plurality compartments, such as a 96-well plate, a 384-well plate, a 1536-well plate; and/or
(ii) generating a sequenceable reaction product in step e) comprises amplifying the barcode label by performing a polymerase chain reaction or an isothermal amplification reaction.
85. The method for analyzing one or more cell released biomolecules according to embodiment 1 or 2, comprising providing a cell-laden matrix, wherein the cell-laden matrix comprises at least one cell that releases one or more biomolecules of interest or wherein the cell-laden matrix comprised at least one cell that has released one or more biomolecules of interest, wherein the method comprises the following steps:
a) providing a capture matrix, wherein the capture matrix comprises one or more types of capture molecules, wherein each type of capture molecule binds a biomolecule of interest;
b) incubating the cell-laden matrix to allow release of the one or more biomolecules of interest and binding the one or more biomolecules of interest to the one or more types of capture molecules of the capture matrix thereby providing a loaded capture matrix;
c) optionally further processing the loaded capture matrix;
d) using a first type of detection molecule comprising a barcode label which comprises a barcode sequence (B_{S}) indicative for a first target biomolecule of interest for generating an amplifiable molecule that comprises the barcode sequence (B_{S}) and/or reverse complement thereof,
wherein step d) comprises
(aa) adding a first type of tagging molecule, that specifically binds the first target biomolecule of interest that is captured by the capture molecule of the capture matrix to the capture matrix,
(bb) adding the first type of detection molecule wherein the first type of detection molecule comprises the barcode label and a binding moiety for binding the first type of tagging molecule and binding the first type of detection molecule to the tagging molecule.
86. The method according to embodiment 85, wherein step d) (aa) and d) (bb) are performed sequentially.
87. The method according to embodiment 85, wherein step d) (aa) and d) (bb) are performed simultaneously, by combining the first type of tagging molecule with the first type of detection molecule thereby forming a conjugate, prior to contacting said conjugate comprising the first type of tagging molecule and the first type of detection molecule with the loaded capture matrix.
88. The method according to any one of embodiments 85 to 87, for analyzing two or more biomolecules of interest, wherein the method comprises
   performing one or more wash steps to remove unbound first type of detection molecules and unbound first type of tagging molecules, and wherein the method comprises
d)′ using a second type of detection molecule comprising a barcode label which comprises a barcode sequence (B_{S}) indicative for a second target biomolecule of interest for generating an amplifiable molecule that comprises the barcode sequence (B_{S}) and/or reverse complement thereof,
wherein step d)′ comprises
(aa) adding a second type of tagging molecule, that specifically binds the second target biomolecule of interest that is captured by the capture molecule of the capture matrix to the capture matrix,
(bb) adding the second type of detection molecule wherein the second type of detection molecule comprises the barcode label comprising the barcode sequence (B_{S}) indicative for a second target biomolecule of interest and a binding moiety for binding the second type of tagging molecule and binding the second type of detection molecule to the tagging molecule;
optionally repeating these steps for detecting further target biomolecules of interest, wherein for each repetition
   a different type of tagging molecule is used and wherein each type of tagging molecule specifically binds to a different target biomolecule of interest; and
   a different type of detection molecule is used, wherein each type of detection molecule comprises a different barcode label that comprises at least a different barcode sequence (B_{S}) that is indicative for a different target biomolecule of interest.
89. The method according to embodiment 88, wherein after adding two or more types of tagging molecules and two or more types of detection molecules, complex is provided that comprises the loaded capture matrix, the two or more types of tagging molecules, wherein each type of tagging molecule binds its target analyte that is bound to the capture matrix, the corresponding to or more types of detection molecules.
90. The method according to any one of embodiments 85 to 89, wherein the tagging molecule comprises a binding molecule that is capable of binding the target biomolecule of interest, optionally wherein the binding molecule of the tagging molecule is selected from the group consisting of antibodies, intact antibodies having full length heavy and light chains, one or more antibody fragments, such as Fab fragments, Fab′, F(ab′)2, Fv, single domain antibodies, scFv, bi-, tri- or tetra-valent antibodies, Bis-scFv, diabody, triabody, tetrabody and epitope-binding fragments thereof.
91. The method according to any one of embodiments 85 to 90, wherein the one or more types of detection molecules used comprise a binding moiety that is capable of binding the corresponding tagging molecule.
92. The method according to any one of embodiments 85 to 91, wherein one or more types of tagging molecules comprise a common interaction moiety that is the same for all types of tagging molecules and wherein the one or more types of detection molecules comprise a common binding moiety that recognizes and binds the common interaction moiety, optionally wherein the correct assignment of each type of tagging molecule to its type of detection molecule comprising a barcode label comprising a barcode sequence B_{S} that is indicative for the target analyte that is bound by the binding molecule of said type of tagging molecule is achieved by sequentially adding different types of tagging molecules and different types of detection molecules and performing one or more wash steps in-between.
93. The method according to any one of embodiments 85 to 92, wherein the tagging molecule comprises an interaction moiety that is selected from an Fc-region of an antibody, a biotin binding polypeptide and biotin.
94. The method according to embodiment 92 or 93, wherein the interaction moiety of the tagging molecule and the binding moiety of the detection molecule that binds said interaction moiety form part of an interaction pair, wherein said interaction pair is preferably selected from:
(i) biotin and a biotin binding polypeptide, wherein the biotin binding polypeptide is optionally selected from tamavidin, streptavidin and avidin;
(ii) the Fc region of an antibody molecule and an Fc region binding protein, wherein the Fc region binding protein is optionally selected from an anti-Fc antibody, an anti-Fc binding antibody fragment and a nanobody;
(iii) an antigen and an antigen binding polypeptide, wherein the antigen binding polypeptide is preferably selected from an antibody, an antibody binding fragment and a nanobody.
95. The method according to any one of embodiments 85 to 94, wherein more than one type of tagging molecule is provided, wherein each type of tagging molecule has a corresponding type of detection molecule and wherein different types of tagging molecules have different types of detection molecules, wherein the different types of detection molecules at least differ in the barcode sequence B_{S}.
96. The method according to any one of embodiments 85 to 95, comprising
e) using the amplifiable molecule for generating a sequenceable product, optionally wherein step e) comprises performing a polymerase chain reaction or an isothermal amplification reaction.
97. The method according to any one of embodiments 85 to 96, having the following features:
(i) step c) comprises
   obtaining the capture matrix from a compartment of a cell culture device, which preferably is a microfabricated cell culture device, and
   transferring the capture matrix comprises transferring the capture matrix to a compartment of a processing device, which preferably is a microfabricated processing device;
(ii) the tagging molecule of step d) comprises an Fc-region;
(iii) the detection molecule comprises a Fc-binding molecule, in particular an anti-Fc antibody or anti-Fc antibody fragment;
(iv) one or more washing steps are performed to remove unbound tagging molecules and unbound detection molecules.
98. The method according to any one of embodiments 85 to 96, having the following features:
(i) step c) comprises
   obtaining the capture matrix from a compartment of a cell culture device, which preferably is a microfabricated cell culture device, and
   transferring the capture matrix comprises transferring the capture matrix to a compartment of a processing device, which preferably is a microfabricated processing device;
(ii) the tagging molecule comprises a biotin binding polypeptide, preferably tamavidin or streptavidin;
(iii) the detection molecule comprises biotin as binding moiety;
(iv) one or more washing steps are performed to remove unbound tagging molecules and unbound detection molecules.
99. The method according to embodiment 97 or 98, wherein the method fulfills one or more of the following characteristics:
(i) the barcode label of the detection molecule comprises:
(aa) a barcode sequence (B_{S}) for indicating the specificity;
(bb) a barcode sequence (B_{T}) for indicating a time information, and
(cc) a barcode sequence (B_{P}) for indicating a position information, and
(dd) optionally a unique molecular identifier (UMI) sequence;
(ii) the method comprises transferring the capture matrix to another compartment before generating a sequenceable reaction product in step e), wherein the compartment is provided by another device comprising a plurality compartments, such as a 96-well plate, a 384-well plate, a 1536-well plate;
(iii) generating a sequenceable reaction product comprises amplifying the barcode label in step e) by performing a polymerase chain reaction or an isothermal amplification reaction.
100. The method according to any one of embodiments 1 to 99, wherein for each target biomolecule of interest analysed in said method a different amplifiable molecule is generated, wherein the amplifiable molecule at least differs in the barcode sequence (B_{S}) that is indicative for a target biomolecule of interest.
101. The method according to any one of embodiments 2 to 100, wherein the sequenceable product generated in step e) comprises a barcode sequence (B_{T}) for indicating a time information and/or a barcode sequence (B_{P}) for indicating a position information.
102. The method according to embodiment 101, wherein the barcode sequence (B_{T}) for indicating a time information and/or the barcode sequence (B_{P}) for indicating a position information is introduced during step e) or preferably, via the barcode label of the detection molecule.
103. The method according to any one of embodiments 1 to 102, wherein step c) is performed and comprises
   obtaining the capture matrix from a compartment of a cell culture device, which preferably is a microfabricated cell culture device, and
   transferring the capture matrix comprises transferring the capture matrix to a compartment of a processing device, which preferably is a microfabricated processing device.
104. The method according to any one of embodiments 1 to 103, wherein step c) is performed and comprises transferring the capture matrix via a fluidic connection between the cell culture device and the processing device, in particular by providing a tube-like connection.
105. The method according to any one of the preceding embodiments, wherein step c) is performed and comprises transferring the capture matrix to a compartment of a processing device, wherein each compartment of the processing device comprises one or more, preferably one, capture matrix.
106. The method according to any one of the preceding embodiments 103 to 105, wherein after transferring the capture matrix in step c) to the compartments of the processing device, the compartments are perfused, wherein compartments of the processing device are individually perfusable.
107. The method according to embodiment 106, wherein the processing device is fluidically connected to another device comprising a plurality compartments, wherein compartments of the plurality of compartments comprise components with which the compartments of the processing device are individually perfusable.
108. The method according to any one of the preceding embodiments, wherein the barcode label of the detection molecule further comprises a barcode sequence (B_{T}) for indicating a time information and wherein n cycles of the present method are performed at different time points tₓ, wherein n is at least 2 and x indicates the different time points, and wherein for each cycle the barcode label differs in its barcode sequence B_{T} from the barcode sequence B_{T} of all other performed cycles.
109. The method according to any one of the preceding embodiments, wherein the barcode label of the detection molecule comprises a barcode sequence B_{P} for indicating position information of a cell-laden matrix analysed, wherein the barcode label of the detection molecule for a cell-laden matrix comprised in a compartment differs in its barcode sequence B_{P} from the barcode sequence B_{P} of a barcode label of a detection molecule for a cell-laden matrix comprised in another compartment.
110. The method according to any one of the preceding embodiments, wherein a plurality of cell-laden matrices and capture matrices are provided in a cell culture device comprising a plurality of compartments, wherein at least one cell-laden matrix and at least one capture matrix are provided within a compartment of the cell culture device.
111. The method according to any one of the preceding embodiments, wherein the barcode label of the detection molecule comprises a unique molecular identifier (UMI) sequence, optionally wherein the UMI sequence has a length of up to 40 nucleotides, optionally 4-20 nucleotides.
112. The method according to any one of the preceding embodiments, wherein
   one or more cells of the at least one cell of the cell-laden matrix is/are inactivated or inactivates, in particular by apoptosis, and/or
   one or more cells of the at least one cell of the cell-laden matrix exit(s) the cell-laden matrix, in particularly by migration.
113. The method according to any one of the preceding embodiments, having one or more of the following characteristics:
   the one or more cells is/are inactivated or inactivate(s) and/or exit(s) the cell-laden matrix before, during and/or after one or more steps of the method are performed,
   the one or more cells is/are inactivated by an external stimulus, optionally provided by a cell culture device, which preferably is a microfabricated cell culture device;
   the one or more cells inactivate(s) induced by an external stimulus, preferably provided by a cell culture device, which preferably is a microfabricated cell culture device;
   the one or more cells undergo(es) apoptosis before, during and/or after performing the method steps;
   the one or more cells exit(s) the cell-laden matrix induced by an external stimulus, preferably provided by a cell culture device, which preferably is a microfabricated cell culture device; and/or
   the one or more cells exit(s) the cell-laden matrix by migration before, during and/or after performing the steps of the method.
114. The method according to any one of the preceding embodiments, wherein steps of the method are performed in a cell culture device that is present in an incubation chamber, which provides conditions suitable for cell cultivation.
115. The method according to any one of the preceding embodiments, wherein steps of the method are performed using a processing device that is accessible by a pipetting device, preferably a pipetting robot and/or wherein method steps, such as method steps a) to d) and preferably e) to g) can be performed automatically.
116. The method according to any one of the preceding embodiments, having one or more of the following features
   a. the matrix comprising at least one cell has one or more of the following characteristics:
      (i) the matrix material is provided by a hydrogel;
      (ii) the matrix is three-dimensional;
      (iii) the matrix is a particle, optionally a hemi-spherical particle or preferably a spherical particle;
      (iv) the matrix has a diameter of ≤1000 μm, such as ≤800 μm, ≤600 μm, or ≤400 μm, preferably ≤200 μm, such as 5 μm to 150 μm; and/or
(vi) the matrix has a volume of ≤200 μl, such as ≤100 μl, ≤50 μl, ≤10 μl, ≤1 μl, ≤0.5 μl, ≤300 nl, ≤200 nl, ≤100 nl, ≤50 nl or ≤5 nl, preferably 0.05 μl to 2000 μl;
   b. the capture matrix comprising the one or more types of capture molecules has one or more of the following characteristics:
      (i) it is a polymer matrix, optionally comprising or consisting of polyacrylamide (PMA), polyactic acid (PLA), poly(vinyl alcohol) (PVA), polyethylene glycol (PEG), polyoxazoline (POx), and polystyrene (PS).
      (ii) the matrix material is provided by a hydrogel;
      (iii) the matrix is three-dimensional;
      (iv) the matrix is a particle, preferably a spherical particle; and/or
      (v) the matrix has a diameter of ≤1000 μm, such as ≤800 μm, ≤600 μm or ≤400 μm, preferably ≤200 μm, such as 5 μm to 150 μm;
      and/or
   c. the cell-laden matrix and the capture matrix are provided in proximity within a compartment of a device or the cell-laden matrix and the capture matrix are provided in separate compartments, wherein the separate compartments are in fluid communication with each other or can be brought in fluid communication with each other so that the released biomolecules of interest can contact the capture matrix.
117. The method according to any one of the preceding embodiments, wherein the matrix of the cell-laden matrix is a hydrogel which has one or more of the following characteristics:
a. the hydrogel comprises cross-linked hydrogel precursor molecules of the same type or of different types;
b. the hydrogel is composed of at least two different polymers with different structures as hydrogel precursor molecules, wherein optionally, at least one polymer is a copolymer;
c. the hydrogel is formed using at least one polymer which has a linear structure and at least one polymer which has a multiarm or star-shaped structure;
d. the hydrogel is formed using a t least one polymer of formula (P1)

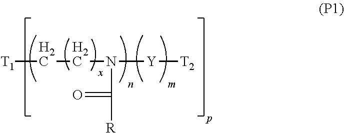

wherein
R is independently selected from a hydrogen atom, a hydrocarbon with 1-18 carbonatoms (preferably CH₃, —C₂H₅), a C₁-C₂₅-hydrocarbon with at least one hydroxy group, a C₁-C₂₅-hydrocarbon with at least one carboxy group, (C₂-C₆)alkylthiol, (C₂-C₆)alkylamine, protected (C₂-C₆)alkylamine (preferably —(CH₂)₂₋₆—NH—CO—R (with R=tert-Butyl, perfluoroalkyl)), (C₂-C₆)alkylazide, polyethylene glycol, polylactic acid, polyglycolic acid, polyoxazoline, or wherein R is a residue R⁴
Y is a moiety containing at least one graft, comprising at least one residue R⁴,
T₁ is a terminating moiety, which may contain a residue R⁴,
T₂ is a terminating moiety, which contains a residue R⁴,
p is an integer from 1 to 10,
n is an integer greater than 1 and preferably, below 500,
m is zero or an integer of at least, preferably greater than 1, and preferably, below 500, the sum n+m is greater than 10,
x is independently 1, 2 or 3, preferably x is independently 1 or 2, most preferably x is 1,
R⁴ independently comprise at least one functional group
   for crosslinking and/or
   for binding biologically active compounds, and
optionally comprising a (preferably degradable) spacer moiety connecting said functional group with the binding site to the respective moiety of the structure of formula (P1), wherein the entirety of all m-fold and n-fold repeating units are distributed in any order within the polymer chain and wherein optionally, the polymer is a random copolymer or a block copolymer.
118. The method according to any one of the preceding embodiments, wherein the method is performed by utilizing a cell culture device, which preferably is a microfabricated cell culture device, wherein the device has one or more of the following features:
   i) at least one compartment for accommodating at least one, preferably at least two matrices, including at least one capture matrix and/or at least one cell-laden matrix;
   ii) at least one compartment that is capable of being switched between an isolated and an open state, wherein the isolated state corresponds to a state at which fluid that is present in the compartment is in no contact with fluid not present in the compartment and wherein the open state corresponds to a state at which fluid that is present in the compartment is in contact with fluid not present in the compartment;
   iii) a compartment for accommodating at least one matrix, preferably two matrices, wherein a microfabricated geometry for matrix immobilization is present suitable for positioning the at least one matrix;
   iv) a plurality of compartments for accommodating at least one matrix, preferably provided by an array of compartments;
   v) a microfabricated valve capable of switching the compartment to an open or closed state;
   vi) a microfabricated valve, comprising a first channel, a second channel, a connection channel connecting the first channel and the second channel, a valve portion arranged within the connection channel, wherein the valve portion is adapted to selectively open and close the connection channel;
   vii) a microfabricated valve comprising at least three layers, wherein a first channel is located within a first layer; a second channel is located within a third layer; a valve portion is located within a second layer; the second layer is arranged between the first and the third layer;
   viii) a microfabricated valve wherein a first channel comprises a microfabricated geometry for matrix immobilization suitable for positioning at least one matrix being contained in a fluid which flows through the first channel, wherein the microfabricated geometry for matrix immobilization is arranged within the first channel in such a way that a fluid flow can be reduced by the microfabricated geometry for matrix immobilization, in particular, the microfabricated geometry for matrix immobilization narrows the cross section of the channel; and/or
wherein a second channel comprises a microfabricated geometry for matrix immobilization suitable for positioning particles being contained in a fluid which flows through the second channel, wherein the microfabricated geometry for matrix immobilization is arranged within the second channel in such a way that a fluid flow can be reduced by the microfabricated geometry for matrix immobilization, in particular, the microfabricated geometry for matrix immobilization narrows the cross section of the channel;
and/or
   ix) a fluid reservoir and fluid channels for providing fluid to the compartment.
119. The method according to any one of the preceding embodiments, wherein the method is performed by utilizing a cell culture device, which preferably is a microfabricated cell culture device, wherein the device comprises one or more of the following features:
   i) at least one matrix is releasably positioned by a preferably microfabricated geometry for matrix immobilization inside a compartment;
   ii) at least one matrix is releasably positioned by a preferably microfabricated geometry for matrix immobilization inside a compartment, wherein the geometry for matrix immobilization has one or more of the following characteristics:
      it is capable of positioning the cell-laden matrix and the capture matrix in proximity;
      it is capable of positioning at least two cell-laden matrix and the capture matrix in proximity;
   iii) at least one cell-laden matrix and at least one capture matrix are positioned by a preferably microfabricated geometry for matrix immobilization inside a compartment, wherein the compartment accommodating the at least one cell-laden matrix is different from the compartment accommodating the at least one capture matrix and wherein both compartments can be switched to be either in fluid contact with other or to be in no fluid contact with each other;
and/or
   iv) it comprises a trapping geometry comprising a valve arrangement adapted to provide a fluid passing through a microfabricated geometry for matrix immobilization wherein the valve arrangement is adapted to selectively change the direction of fluid passing the microfabricated geometry for matrix immobilization, in particular wherein a fluid a first direction urging the at least one matrix into the microfabricated geometry for matrix immobilization and a fluid in the second direction urging the at least one matrix out of the microfabricated geometry for matrix immobilization, and in particular fluid in the second direction delivering the at least one matrix in direction of an exit section.
120. A kit comprising one or more components used in the methods according to any one of embodiments 1 to 119.
121. A kit according to embodiment 120, comprising one or more types of detection molecules and/or types of oligonucleotides as defined in any one of embodiments 3 to 37 or 44 to 50.
122. A kit according to embodiment 120 or 121, comprising an oligonucleotide attached to the capture matrix and the capture molecule as defined in any one of embodiments 38 to 50 and/or one or more types of detection molecules as defined in any one of embodiments 3 to 37 or 44 to 50.
123. A kit according to any one of embodiments 120 or 122, comprising a capture matrix and one or more types of detection molecules as defined in any one of embodiments 51 to 70.
124. A kit according to any one of embodiments 120 or 123, comprising a capture matrix, one or more types of detection molecules and optionally a tagging molecule as defined in any one of embodiments 71 to 84.
125. A kit according to any one of embodiments 120 or 124, comprising a capture matrix, one or more types of detection molecules and a tagging molecule as defined in any one of embodiments 85 to 99.

### The Kit According to the Second Aspect

According to a second aspect, the present disclosure provides one or more kits comprising one or more components used in the methods according to the present invention. Such kits are also disclosed in the above embodiments 120-125. The kits can be used e.g. in the methods according to the present invention.

The wash solutions, such as washing buffers may be used for removing unbound analytes and detection molecules.

According to one embodiment, the kit comprises a device with a plurality of compartments, preferably a multi-well plate (e.g. 96, 384 or 1536 well plate). The compartments of the device may comprise at least one oligonucleotide, a detection molecule, and/or a primer or primer combination. Details are described elsewhere herein and it is referred to the respective disclosure. The compartments may furthermore comprise reagents for performing an extension and/or amplification reaction.

The kit may comprise a device with a plurality of compartments, preferably a multi-well plate, wherein said device has one or more of the following characteristics. The compartments may furthermore comprise reagents for performing an extension and/or amplification reaction, such as an enzyme mix comprising a reverse transcriptase (such as M-MuLV or AMV) and/or a polymerase (such as Taq DNA Polymerase) and optionally dNTPs. The reagents may be provided in lyophilized form in the compartments of the well. The device may be e.g. selected from a 96, 384 or 1536 well plate. The kit may furthermore comprise a solution for reconstitution of the lyophilized reagents. The kit may also comprise one or more reaction buffers and nuclease-free water.

1. A method for analyzing one or more cell released biomolecules, comprising providing a cell-laden matrix, wherein the cell-laden matrix comprises at least one cell that releases one or more biomolecules of interest or wherein the cell-laden matrix comprised at least one cell that has released one or more biomolecules of interest, wherein the method comprises the following steps:
a) providing a capture matrix, wherein the capture matrix comprises one or more types of capture molecules, wherein each type of capture molecule binds a biomolecule of interest,
b) incubating the cell-laden matrix to allow release of the one or more biomolecules of interest and binding the one or more biomolecules of interest to the one or more types of capture molecules of the capture matrix thereby providing a loaded capture matrix;
c) optionally further processing the loaded capture matrix;
d) using one or more types of detection molecules comprising a barcode label which comprises a barcode sequence (B_{S}) indicative for a target biomolecule of interest for generating an amplifiable molecule that comprises the barcode sequence (B_{S}) and/or reverse complement thereof.

2. The method according to claim 1, wherein the barcode label of a detection molecule used in step d) comprises at least one nuclease target site (NTS) and an analyte specific sequence (ASS) that is specific for a biomolecule of interest.

3. The method of claim 2, wherein step d) comprises hybridizing the barcode label of the detection molecule to an oligonucleotide that is associated with the loaded capture matrix, wherein said associated oligonucleotide comprises
at least one nuclease target site (NTS′) that is complementary to the at least one nuclease target site (NTS) of the barcode label of the detection molecule, and
at least one analyte specific sequence (ASS′) that is complementary to the analyte specific sequence (ASS) of the barcode label of the detection molecule,
whereby an at least partially double-stranded hybrid molecule is formed that comprises (i) at least one cleavable recognition site for an endonuclease that is provided by the hybridized nuclease target sites and (ii) the hybridized analyte specific sequences.

4. The method of claim 3, wherein in the barcode label, the analyte specific sequence (ASS) is arranged 3′ to the at least one nuclease target site (NTS) and wherein in the oligonucleotide, the analyte specific sequence (ASS′) is arranged 5′ to the at least one nuclease target site (NTS′).

5. The method according to any one of claims 2 to 4, wherein the barcode label of the detection molecule comprises a blocked 3′OH end which prevents elongation of the barcode label at the 3′ end.

6. The method according to any one of claims 2 to 5, wherein the barcode label of the detection molecule comprises
(i) the at least one nuclease target site (NTS);
(ii) an analyte specific sequence (ASS) which is located 3′ to the at least one nuclease target site (NTS);
(iii) the barcode sequence (B_{S}) which is located 5′ to the at least one nuclease target site (NTS);
and wherein the barcode label additionally comprises
(iv) optionally a sequencing primer target sequence, which is located 5′ to the barcode sequence (B_{S});
(v) a primer target sequence, preferably a universal primer target sequence, which is located 5′ to the barcode sequence (B_{S}) and, if present, 5′ to the sequencing primer target sequence;
(vi) optionally a universal adapter sequence, which is located 5′ to the at least one nuclease target site (NTS) and 3′ to the barcode sequence (B_{S}); and
(vii) optionally a unique molecular identifier (UMI) sequence, which is located 5′ to the at least one nuclease target site (NTS) and, if present, 5′ to the universal adapter sequence.

7. The method according to claim 6, wherein the barcode label additionally comprises 5′ to the at least one nuclease target site (NTS) and, if present, 5′ to the universal adapter sequence a barcode sequence (B_{T}) for indicating a time information and/or a barcode sequence (B_{P}) for indicating a position information.

8. The method according to any one of claims 3 to 7, wherein step d) comprises cleaving the at least partially double-stranded hybrid molecule at the at least one cleavable recognition site using an endonuclease that recognizes said recognition site thereby providing a cleaved barcode label comprising a free 3′OH end at the cleaved nuclease target site (NTS*).

9. The method according to claim 8, wherein step d) comprises extending the cleaved and optionally further processed barcode label in an extension reaction at its 3′ end to provide an amplifiable molecule that comprises at least one sequence from the extension reaction.

10. The method according to claim 9, wherein said amplifiable molecule is provided in the extension reaction by hybridizing an extension oligonucleotide to the cleaved and optionally further processed barcode label, wherein the extension oligonucleotide provides a template for the extension reaction,
wherein preferably, an amplifiable molecule is generated in the extension reaction which includes a universal primer target sequence added at the 3′ end of the barcode label, wherein such amplifiable molecule is provided by hybridizing an extension oligonucleotide to the cleaved and optionally further processed barcode label, wherein the extension oligonucleotide provides the template for adding in the extension reaction the universal target primer sequence at the 3′ end of the barcode label.

11. The method according to claim 9 or 10, in particular when dependent on claim 6, characterized in that
(a) the extension oligonucleotide has one or more of the following characteristics:
(i) it comprises a blocked 3′OH end which prevents elongation of the extension oligonucleotide,
(ii) it comprises one or more sequences complementary to one or more sequences of the cleaved barcode label,
(iii) it comprises a universal primer target sequence,
(iv) it comprises one or more nucleotides that is/are complementary to the nucleotides of the cleaved nuclease target site (NTS*) of the cleaved barcode label;
(b) the barcode label comprises the universal adapter sequence and wherein the extension oligonucleotide comprises a sequence that is complementary to the universal adapter sequence;
(c) the barcode label comprises the universal adapter sequence and wherein the extension oligonucleotide hybridizes to the cleaved nuclease target site (NTS*) and the universal adapter sequence of the cleaved barcode label;
(d) the method comprises releasing the associated oligonucleotide from the loaded capture matrix prior to performing the hybridization reaction with the barcode label;
(e) the method comprises separating cleaved barcode labels from intact and thus uncleaved barcode labels; and/or
(f) the method comprises obtaining the cleaved barcode labels and transferring the cleaved barcode labels to a compartment of a device comprising a plurality compartments, such as a 96-well plate, a 384-well plate, a 1536-well plate.

12. The method according to any one of claims 9 to 11 when dependent on claim 6, wherein the barcode label comprises the universal adapter sequence and wherein the extension reaction provides an amplifiable molecule which comprises from 5′ to 3′ a universal primer target sequence, optionally a sequencing primer target sequence, the barcode sequence (B_{S}), the universal adapter sequence, optionally the cleaved nuclease target site (NTS*), and the universal primer target sequence added in the extension reaction, optionally wherein the amplifiable molecule additionally comprises
a unique molecular identifier (UMI) sequence,
a barcode sequence (B_{T}), and
a barcode sequence (B_{P}),
located 5′ to the universal adapter sequence and 3′ to the universal primer target sequence and, if present, 3′ to the sequencing primer target sequence.

13. The method according to any one of claims 1 to 12, comprising
e) using the amplifiable molecule for generating a sequenceable product, optionally wherein step e) comprises performing a polymerase chain reaction or an isothermal amplification reaction;
f) optionally sequencing the sequenceable product generated in step e) or the amplifiable molecule generated in step d), and
g) optionally evaluating the sequencing data obtained in step f), wherein evaluating preferably comprises analyzing the obtained sequencing data to determine the presence or absence of the one or more target biomolecules of interest.

14. The method according to claim 12 and 13, comprising
e) generating a sequenceable product by amplifying the amplifiable molecule using a primer or primer combination,
optionally wherein a primer combination is used, wherein said primer combination comprises a first universal primer that hybridizes to the added universal primer sequence located 3′ to the cleaved nuclease target site (NTS*) and wherein a second universal primer hybridizes to the reverse complement of the universal primer sequence located at the 5′ end of the amplifiable molecule.

15. The method according to any one of claims 6 to 9 when depending on claim 6, wherein the barcode label of the detection molecule comprises a universal primer target sequence which is located 5′ to the barcode sequence (B_{S}) and, if present, 5′ to the sequencing primer target sequence and wherein said barcode label comprises a free 3′OH end and a further universal primer target sequence 5′ to the at least one nuclease target site (NTS) and 3′ to the barcode sequence (B_{S}).

16. The method according to claim 15 when dependent on claim 3, wherein step d) comprises
hybridizing the barcode label of the detection molecule to the oligonucleotide that is associated with the loaded capture matrix whereby an at least partially double-stranded hybrid molecule is formed that comprises in a double-stranded portion of said molecule (i) at least one cleavable recognition site for an endonuclease and (ii) the hybridized analyte specific sequences, and
cleaving the at least partially double-stranded hybrid molecule at the at least one cleavable recognition site using an endonuclease that recognizes said recognition site thereby releasing an amplifiable product comprising a free 3′OH end and the remaining sequences of the barcode label including two universal primer target sites that flank at least the comprised barcode sequence(s),
wherein the method comprises separating the cleaved barcode labels of the detection molecules providing the amplifiable product from uncleaved detection molecules, e.g. by obtaining the cleaved barcode labels and transferring the cleaved barcode labels to a compartment of a device comprising a plurality compartments, such as a 96-well plate, a 384-well plate, a 1536-well plate.

17. The method according to claim 16, wherein the method comprises
e) generating a sequenceable product by amplifying the amplifiable molecule using a universal primer or primer combination capable of hybridizing to at least one of the universal primer target sites of the amplifiable product,
optionally wherein a first universal primer hybridizes to the universal primer sequence that was in the barcode label located 5′ to the nuclease target site (NTS) and wherein a second universal primer hybridizes to the reverse complement of the universal primer sequence located at the 5′ end of the amplifiable molecule, wherein the first and second universal primers are the same of different.

18. The method according to any one of claims 2 to 17, wherein the barcode label comprises two or more nuclease target sites (NTS), optionally wherein the two or more nuclease target sites (NTS) provide when present in a double-stranded hybrid recognition sites for two or more different endonucleases.

19. The method according to claim 18, wherein the two or more nuclease target sites (NTS) are collocated in the barcode label, optionally separated by one or more further sequences, wherein if present, the associated oligonucleotide comprises sequences complementary thereto to facilitate hybridization.

20. The method according to claim 19, wherein the analyte specific sequences (ASS) is located in-between two nuclease target sites (NTS).

21. The method according to any one of claims 18 to 20 when dependent on claim 3, characterized by one or more of the following features:
(aa) two or more cleavable recognition sites for different endonucleases are present in the at least partially double-stranded hybrid and step d) comprises cleaving the at least partially double-stranded hybrid molecule using two or more different endonucleases that recognizes said recognition sites;
(bb) the endonuclease is a restriction endonuclease;
(cc) the endonuclease is a restriction endonuclease that recognizes a specific nucleotide sequence as cleavable recognition site in the double-stranded DNA hybrid that is formed between the barcode label and the oligonucleotide, optionally wherein the restriction endonuclease cuts the cleavable recognition site so that no more than 5 nucleotides, no more than 4 nucleotides, no more than 3 nucleotides or not more than 2 nucleotides of the original nuclease target site remain at the 3′ end of the cleaved nuclease target site (NTS*) of the barcode label;
(dd) the endonuclease is a type II restriction endonuclease;
(ee) the endonuclease is a commercially available restriction endonuclease, optionally selected from BamHI, BgIII, ClaI, EcoRI, HindIII, PstI, SalI and XmaI.

22. The method according to any one of claims 13 to 21, comprising
f) sequencing the sequenceable product generated in step e).

23. The method according to any one of claims 3 to 22, wherein the barcode label of the detection molecule hybridizes to the oligonucleotide, wherein hybridization involves the analyte specific sequence (ASS), wherein
the barcode labels of one type of detection molecule are the same, thereby hybridizing to oligonucleotides of the matching type of oligonucleotide, and
wherein the barcode labels of another type of detection molecule is different at least in the analyte specific sequence (ASS), thereby hybridizing to the oligonucleotides of another type of oligonucleotides comprising matching sequences complementary to the analyte specific sequence (ASS) of the matching type of detection molecule.

24. The method according to one of claims 3 to 23, comprising using two or more types of detection molecules, wherein each type of detection molecule binds to one type of oligonucleotide, in particular by hybridizing via the analyte specific sequence (ASS) of the barcode label to the complementary sequence (ASS′) of the matching type of oligonucleotide, wherein each type of detection molecule comprises a different analyte specific sequence (ASS) and each matching type of oligonucleotide comprises a hybridizing complementary sequence (ASS′).

25. The method according to any one of claims 3 to 24, wherein the barcode label comprised in one type of detection molecule is the same and wherein the barcode label comprised in another type of detection molecule differs in
the barcode sequence (B_{S}) for indicating the specificity, and
analyte specific sequence (ASS).

26. The method according to one or more of claims 3 to 25, wherein different types of detection molecules used at the same time comprise barcode labels that share
(ii) a common a barcode sequence (B_{T}) for indicating a time information, and
(iii) a common barcode sequence (B_{P}) for indicating a position information.

27. The method according to claim 26, wherein at least all barcode labels of the same type of detection molecule comprise a unique molecular identifier (UMI) sequence.

28. The method according to any one of claims 3 to 27 when dependent on claim 3, wherein the oligonucleotide is attached to a secondary capture molecule that binds a biomolecule of interest that is bound by a capture molecule of the capture matrix and wherein the oligonucleotide is associated with the loaded capture matrix by means of said secondary capture molecule.

29. The method of claim 28, characterized by one or more of the following features:
(aa) the oligonucleotide is attached via a linker to the secondary capture molecule;
(bb) the oligonucleotide comprises a blocked 3′OH end which prevents elongation of the oligonucleotide;
(cc) the secondary capture molecule is selected from an antibody or antibody binding fragment.

30. The method of claim 28 or claim 29, wherein the secondary capture molecule comprising the oligonucleotide is added in step c) in order to allow binding of the secondary capture molecule to its biomolecule of interest that is bound by the loaded capture matrix whereby the oligonucleotide becomes associated with the loaded capture matrix.

31. The method according to claim 30, wherein step c) comprises washing the loaded capture matrix with the bound secondary capture molecule comprising the oligonucleotide and optionally transferring the loaded capture matrix with the associated oligonucleotide to a new partition.

32. The method according to any one of claims 28 to 31, wherein the detection molecule comprises or consists of a single-stranded oligonucleotide.

33. The method according to any one of claims 13 to 32, when dependent on claim 3, wherein step e) comprises performing an amplification reaction using a primer or primer combination, preferably wherein the primer or primer combination hybridizes to the cleaved or cleaved and extended barcode label and/or a complement thereof.

34. The method according to any one of claims 13 to 33, when dependent on claim 3, wherein generating a sequenceable reaction product in step f) comprises amplifying the cleaved or cleaved and extended barcode label by performing a polymerase chain reaction or an isothermal amplification reaction.

35. The method according to any one of claims 13 to 34, when dependent on claim 3, wherein generating a sequenceable reaction product comprises providing one or more primers, preferably a primer pair, optionally wherein the one or more primers hybridize to the cleaved or cleaved and extended barcode label or a complement thereof.

36. The method according to claim 35, wherein the one or more primers comprise one or more barcode sequences, wherein the barcode sequences are present on the sequenceable reaction product after generating the sequenceable product.

37. The method according to claim 36, wherein the one or more primers comprise a barcode sequence (B_{T}) for indicating a time information; and/or the one or more primers comprise a barcode sequence (B_{P}) for indicating a position information.
